(19) United States
(12) Patent Application Publication
(10) Pub. No.: US 2026/0256989 A1
(43) Pub. Date: Sep. 3, 2026

(54) LIGHT BEAM PROJECTION GUIDED BIOFABRICATION OF ALIGNED TISSUES

(71) Applicant: ETH ZÜRICH, Zürich (CH)

(72) Inventors: Marcy ZENOBI-WONG, Zürich (CH); Parth CHANSORIA, Zürich (CH); Hao LIU, Zürich (CH); Jakub JANIAK, Zürich (CH); Ricardo RIZZO, Zürich (CH)

(73) Assignee: ETH ZÜRICH, Zürich (CH)

(21) Appl. No.: 18/879,823

(22) PCT Filed: Jun. 28, 2023
(86) PCT No.: PCT/EP2023/067748

§ 371 Date: Dec. 30, 2024

(30) Foreign Application Priority Data
Jun. 28, 2022 (EP) 22181705.9

(51) Int. Cl. A61L 27/52; A61F 2/08; A61L 27/36

(52) U.S. Cl. CPC A61L 27/52; A61F 2/08; A61L 27/3604; A61F 2002/0894; A61F 2240/001

(57) ABSTRACT

One aspect of the invention relates to a method for making a three-dimensional hydrogel bioimplant, characterized by geometrical structures in sub-millimeter size range. The method comprises the provision of a first composition susceptible to photo-crosslinking in a container. This first composition comprises a first polymer susceptible to photo-crosslinking, a photoinitiator and optionally, a refractive index matching agent, a photo absorptive dye, and a crosslinking agent. In a first illumination step, the composition is illuminated with a plurality of spatially coherent light beams, whereby a plurality of micropillars is generated in the composition. Another aspect of the invention relates to a three-dimensional hydrogel implant comprising or essentially consisting of a plurality of microbeams of a photo-crosslinked polymer, said three-dimensional hydrogel implant obtained by a method according to the invention.

The present application claims the benefit of priority from European Application 22181705.9, filed 28 Jun. 2022, which is incorporated herein by reference.

### FIELD

The present invention relates to a method for making a three-dimensional hydrogel bioimplant by illuminating a first composition comprising a polymer susceptible to photo-crosslinking, a photoinitiator and optionally, a refractive index matching agent, a photo absorptive dye, a crosslinking agent with a plurality of spatially coherent light beams, thereby generating a plurality of micropillars in the composition. The invention further relates to bioimplants generated by the method according to the invention.

### BACKGROUND

Light-based projection techniques are increasingly being used for fabrication of biomimetic tissues. Lately, rapid biofabrication of complex cellular architectures has been possible through tomographic projection of laser light beams. However, most light-guided tissue fabrication strategies have limited potential for efficient cell alignment when it comes to the creation of anisotropic tissues such as muscle and tendons, because most approaches focus on macro-features (>100 μm) that lack the topographical cues necessary for the highly aligned cellular and extracellular organization found in these tissues. For techniques such as two-photon polymerization that can achieve cell-scale (<30 μm) resolution, a compromise on the speed and scalability limits the translational potential of these approaches. Instructive guidance cues have been widely studied to advance fabrication and maturation of anisotropic tissues, such as muscle, tendons, and nerves. Topological cues with an increased aspect ratio have been shown to affect the bioactivity of cells in/on the substrate. For example, the rod-shaped microgels (aspect ratio of 10) fabricated by microfluidics or soft lithography have the ability to increase cell orientation, which is better achieved through the void between high aspect ratio micro-rods compared to microspheres. Topological features with ultra-high aspect ratio (>20:1) created by micropatterning techniques can effectively induce cell adhesion and alignment. Particularly when the dimensions of the confinement approach the scale of the cell nucleus (<10 μm), nuclear deformation resulting from these longitudinal confinements becomes apparent. The elongated shape of the cell nucleus can influence cell differentiation, gene expression, and rejuvenation, the latter by chromosome reorganization and activation of DNA repair mechanisms, and a change to a rounder nuclear shape can be associated with disease pathology. At the tissue level, a more elongated cell nucleus (aspect ratio of 2.5-6) can be observed in anisotropic tissues like tendons, compared to isotropic tissues (1.1-1.8). A high aspect ratio of the nucleus in tenocytes is contributed to maintaining the phenotype of tenocytes and the expression of key genes during aging. However, none of the above techniques provide topological cues for both cell alignment and nucleus deformation in a 3D environment.

In this work, the inventors show that the phenomenon of optical modulation instability (OMI) of light beams inside a photoresin can be advantageously employed for the fabrication of highly aligned microbeams (φ<30 μm) inside macro-sized (L≥10 mm, φ≥3 mm) hydrogel constructs, which then can be further used for the fabrication of anisotropic tissues. OMI is defined by the spontaneous break-up of a uniform optical beam into randomly distributed smaller light beams of identical diameter while propagating within an optical nonlinear media. Photoreactive polymers are well-known optically nonlinear media, since their photopolymerization rate and thus their refractive index (RI) change depends on the intensity of the crosslinking light. Highly aligned beam-like microstructures (microbeams) were generated by shining a partially spatially coherent light beam into a resin-containing vial. Upon incidence of the light beam onto the hydrogel interface, individual microbeams are seeded by local intensity maxima of the incident light beam. Each local maximum intensity of light offers a faster crosslinking rate, thus creating a local maximum RI. In turn, local RI maxima act as optical traps, resulting in the propagation of the whole micro-patterned polymerization front through the volume of resin via these self-focusing waveguides, which leads to the permanent recording of light filamentation. At the same time, local minima of light intensity are distributed between the localized light beam, corresponding to the formation of void spaces where local intensity is below the threshold required for polymerization. These voids between hydrogel microbeams are presented as channel-like void spaces (microchannels) with ultra-high aspect ratios after the removal of uncrosslinked photoresin.

Here, the inventors present a biofabrication strategy for anisotropic tissues called Light Beam Projection (LBP), in which highly aligned microbeams are OMI-induced by the interaction of spatially coherent light beams with varying photoresin systems. Cells are rapidly (<10 s) and safely encapsulated into hydrogel matrix containing highly aligned microstructures. This approach meets the requirements for engineering biomimetic anisotropic tissues, including instructive guidance cues at micro-scale resolution and fast and cell-friendly processing. In the LBP process, the dimension of microbeams and the void spaces between the microbeams are tunable and on the same length scale as cells, which provides efficient cell guidance properties and supports cell migration through the inter-microbeam void spaces.

The inventors' strategy provides control of the fabricated hydrogel matrix at the micro- and macro-level; that is, control over the dimension of individual microbeams (2-30 μm) and over the size and shape of projected tissue structure (100 μm-1 cm). These advantages offer flexibility in the biofabrication of cell-laden hydrogel structures, such as multi-hollow/tubular hydrogel structures which have been shown to improve cell viability prior to vascularization. Finally, the inventors show LBP can be efficiently utilized for multi-cellular/multi-material biofabrication. Using a multi-step projection, (bio)photoresins can be cured at desired locations to create complex tissue constructs that better mimic the hierarchical organization of native tissues, such as muscle.

Constantini et al. (Biomaterials 2017, 131, 98-110) disclose the fabrication of artificial skeletal muscle tissue by 3D bioprinting of hydrogel fibres into which muscle precursor cells are embedded.

Parkatzidis et al. (ACS Biomaterials Sci. Eng. 2019, 5, 6161-6170) disclose photostructuring of gelatin methacrylamide-chitosan hydrogels.

Based on the above-mentioned state of the art, the objective of the present invention is to provide means and methods to generate improved bioimplants having internal structural elements of sub-millimetre structural features. This objective is attained by the subject-matter of the independent claims of the present specification, with further advantageous embodiments described in the dependent claims, examples, figures and general description of this specification.

### SUMMARY OF THE INVENTION

One aspect of the invention relates to a method for making a three-dimensional hydrogel bioimplant, characterized by geometrical structures in sub-millimeter size range. The method comprises the provision of a first composition susceptible to photo-crosslinking in a container. This first composition comprises a first polymer susceptible to photo-crosslinking, a photoinitiator and optionally, a refractive index matching agent, a photo absorptive dye, a crosslinking agent. In a first illumination step, the composition is illuminated with a plurality of spatially coherent light beams, whereby a plurality of micropillars is generated in the composition.

Another aspect of the invention relates to a three-dimensional hydrogel implant comprising or essentially consisting of a plurality of microbeams of a photo-crosslinked polymer, said three-dimensional hydrogel implant obtained by a method according to the invention.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising”, “having”, “containing”, and “including”, and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a”, “or” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic, and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term spatially coherent light beams in the context of the present specification relates to a plurality of light beams created from a light source emitting a defined wavelength that can trigger the photocrosslinking (e.g., 405 nm). The light beams are spatially coherent through their generation by a suitable optical system (e.g., Koehler standard illumination system) or a spatially coherent laser. Spatial coherence is the essential prerequisite of the strong directionality of laser beams. Laser have the potential for generating light beams with very high spatial coherence. The light beam has a fixed phase relationship between the electric field at the different spatial positions across the beam profile.

The term thiol-ene photo-crosslinking in the context of the present specification relates to light triggered step-growth reaction between an alkene (-ene) moiety and a thiol group to form a thioether bond. This reaction requires a photoinitiator that generates radical initiator species upon light absorption. The so formed radicals abstract the hydrogen from the thiol leading to a thiyl radical which then attack the double bond of the -ene group, generating a carbon-centered radical. The latter abstracts the hydrogen of another thiol, leaving a formed thioester bond (crosslinking).

The term “micropillar” in the context of the present specification, also referred to as “microfilament” or “microbeam” throughout this specification, relates to a polymeric structure generated by the method of the invention, consisting of the microbeam-induced photopolymerization product of the polymer susceptible to photo-crosslinking as specified here. Typical dimensions of a micropillar are (width): 1-100 μm. Length: 100 μm to 15 cm (i.e. the microfilaments are prevalent through the entire length of the constructs. The term “prevalent” expresses that the micropillars or microfilaments essentially run through the entire length of the constructs). If not specified otherwise, microstructural dimensions given herein are determined by brightfield microscopy and image analysis by ImageJ (public domain, author: Wayne Rasband).

### DETAILED DESCRIPTION OF THE INVENTION

A first aspect of the invention relates to a method for making a three-dimensional hydrogel bioimplant. The implant is characterized by geometrical structures in sub-millimeter size range, which cannot be attained by conventional 3D-printing protocols. Such sub-mm structures are important as cues for tissue growth and organization. The method according to this first aspect comprises a first composition susceptible to photo-crosslinking, which is provided in a container having walls that are suitable for illumination of the first composition by a plurality of light beams of defined wavelength and coherence length.

The first composition comprises a first polymer susceptible to photo-crosslinking and a photoinitiator. Optionally, a crosslinking agent may be present, if necessary to crosslink the polymer, a refractive index matching agent and photo absorptive dye may be applied to improve the quality of projection/multi-step projection when using a polymer solution with higher cell numbers (typically, >10 million cells/mL).

In a first illumination step, the composition is illuminated with a plurality of spatially coherent light beams, thereby generating a plurality of micropillars in the composition.

### The Polymer

In principle, any polymer susceptible to photopolymerization can be employed. In particular embodiments, the composition comprises the polymer in aqueous solution. As the method of the invention is of particular utility for the preparation of cell containing or cell supporting structures, aqueous polymerization is the method of choice.

In certain embodiments, the polymer susceptible to photo-crosslinking is a biopolymer functionalized by covalent attachment of carbon-carbon double bond (ene) containing moieties which can be crosslinked by thiol bearing crosslinking agents; or a methacrylate-functionalized biopolymer that can be reproducibly prepared by the reaction of biopolymer with methacrylic anhydride.

One particular crosslinking agent that has proven useful in the inventors' hands is a thiol-functionalized poly(ethyleneglycol), one particular example of which a pentaerythrol-PEG-thiol [C(CH₂O(CH₂CH₂O)ₙCH₂CH₂SH)₄]. Such linkers are available from Merck-Sigma, and include pentaerythritol tetrakis(3-mercaptopropionate) (Sigma 381462), trimethylolpropane tris(3-mercaptopropionate) (Sigma 381489); 2-hydroxymethoy-2-methyl-1,3-propanediol tris-(3-mercaptoproprionate) (Aldrich S51145).

In the case of a norbornene or acrylate modified polymer, a crosslinking agent providing the thiol groups is required. Alternatively, it is possible to employ two polymers, one with the thiol and the other with the vinyl group. It would also possible to use a polymer bearing both the carbon-carbon double bond “ene” functionality, and SH groups.

In certain embodiments, the biopolymer is selected from the group comprised of gelatin, hyaluronan, alginate, collagen, chitosan, fibrinogen, Polyvinyl alcohol, silk fibroin, cellulose, see Guo et al., ACS Appl. Mater. Interfaces 2021, 13, 6, 7037-7050; Michel et al., ACS Appl. Bio Mater. 2020, 3, 8, 5253-5262. The inventors similarly contemplate de-cellularized extracellular matrix as a possible biopolymer for functionalization.

In certain embodiments, the carbon-carbon double bond (ene) containing moieties are selected from the group comprised of a norbornene carboxylic acid or dicarboxylic acid, methacrylic acid ester or -amide, acrylic acid ester or -amide, and vinyl esters.

In certain particular embodiments, the carbon-carbon double bond (ene) containing moieties are conferred by a norbornene carboxylic acid or dicarboxylic acid.

The invention may be practiced with many norbornene or methacrylic acid derivatives that can be used to graft such reactive groups onto polymers (i.e., alkyne, azide, hydrazide, DBCO etc., and terminated linkers).

In certain embodiments, the polymer susceptible to thiol-ene photo-crosslinking is selected from the group comprised of norbornene-functionalized gelatin, norbornene-functionalized collagen, norbornene-functionalized chitosan, norbornene-functionalized fibrinogen, norbornene functionalized Polyvinyl alcohol, norbornene-functionalized hyaluronan, gelatin methacryloyl (Gel-MA), hyaluronic acid methacryloyl (HA-MA), alginate methacryloyl (Alg-MA).

In certain particular embodiments, the polymer susceptible to thiol-ene photo-crosslinking is norbornene-functionalized gelatin; see Göckler et al., Advanced Healthcare Materials 19 Jun. 2021, (https://doi.orq/10.1002/adhm.202100206).

Similarly, it is possible to use the thiolated derivative of the polymers, in combination with an ene-bearing crosslinker, or combinations of thiol and ene bearing polymers, such as norbornene-functionalized hyaluronan and thiolated alginate, or norbornene-functionalized gelatin and thiolated hyaluronan.

In certain embodiments, the norbornene-functionalized gelatin is characterized by a degree of substitution of 10% to 90%, particularly from 30% to 55%, more particularly from 47% to 50%.

The degree of substitution (DS) of NB-modified gelatin can be defined as the millimoles of norbornene moieties per gram of gelatin. Alternatively, it can be defined as the percentage of lysine groups bearing a norbornene functionality. In both cases the determination is done by 1-H NMR using an internal standard (DSS).

As defined in the materials and method section of Rizzo et al. (ibid.): “Gel-NB degree of substitution (DS) was determined by 1H-NMR (Bruker Ultrashield 400 MHz, 1024 scans). In short, Gel-NB was solubilized at 40 mg mL⁻¹ in a solution of 0.5 mg mL⁻¹ 3-(trimethylsilyl)-1-propanesulfonic acid (DSS) in D₂O (Apollo Scientific). DSS was used as an internal standard to calculate NB millimoles per gram of gelatin by comparing integrals of the DSS nine methyl protons (≈0.5 to −0.5 ppm) with the two NB-ene protons (≈6.21-6.00 ppm) (n=3). DS given as a percentage was calculated based on the lysine+hydroxylysine content of porcine skin gelatin type A (0.325 mmol g⁻¹) estimated by Claaßen et al. (Biomacromolecules 2018, 19, 42-52).

There are no structural constrains for both thiol and alkene, which can react with different kinetics depending on their structure (i.e, norbornene reacts faster than acrylate). This reaction is not limited by solvent, it can be performed either in aqeuous solutions or organic solvents (i.e., DMSO, DMF). The pH of the solution has some influence, since at alkaline pH (>7), thiol reacts with several -ene groups without light through a nucleophilic addition reaction (“Michael addition”). In certain embodiments, norbornene derivatives are used as ene function; the advantage of using norbornene is that it does not undergo this kind of reaction and therefore avoids unintentional, non-photoreaction-triggered crosslinking of the mixed thiol and -ene components.

Therefore, for most -ene functionalities (i.e., vinyl ether, vinyl sulfones, methacrylates, acrylates) it is advised to use pH<7 to avoid undesired crosslinking (meaning thiol-ene reaction happening without the trigger of light). It is also important to note here that using other -ene moieties (like methacrylates) will lead to a network partially formed by kinetic chains (chain-growth polymerization, as for the use of simple Gel-MA), therefore losing some of the advantages of purely photoclick thiol-ene chemistry.

Another way to categorize polymers that can be employed for practicing the invention is as follows:

### Resins Based on Step Growth Photo-Polymerization:

The norbornene-functionalized component is selected from the group consisting of collagen, decellularized matrix, gelatin, hyaluronic acid (HA), polyethylene glycol (PEG), or polyvinyl alcohol (PVA); and a thiolated component for crosslinking is selected from collagen, gelatin, hyaluronic acid, polyethylene glycol (PEG), or polyvinyl alcohol (PVA), or dithiothreitol (DTT).

### Resins Based on Chain Growth Photo-Polymerization:

Methacrylate or acrylate-functionalized collagen, gelatin, hyaluronic acid, polyethylene glycol (PEG), or polyvinyl alcohol (PVA) or alginate.

### Resins not Requiring any Modification:

Collagen, fibrinogen, decellularized matrix, gelatin to be used with the Ru-SPS photoinitiator system (details of photoinitiators are mentioned below). The Ru-SPS system crosslinks the free tyrosine groups found in these matrices, for which reason the mentioned matrices or resins do not necessarily require modification. This Ru-SPS photoinitiator system can also crosslink the acryl groups and norbornene groups, so it can work also with the materials given in the preceding paragraphs.

### Exemplary Concentration of Polymers

Non-limiting values for concentrations that have worked well in the inventors' hands include: collagen: 1-30 mg/ml; fibrinogen: 5-100 mg/ml; decellularized matrix: 5-100 mg/ml; gelatin: 5-100 mg/ml; PVA: 1-30 mg/ml; PEG: 5-200 mg/ml; HA: 1-30 mg/ml)

### The Photoinitiator

Any photoinitiator In certain embodiments, the photoinitiator is selected from the group comprised of lithium phenyl-2,4,6-trimethylbenzoylphosphinate (LAP), 2,4,6-trimethylbenzoyldiphenylphosphine oxide (TPO), bis-acylphosphine oxide (BAPO), 2-hydroxy-4′-(2-hydroxyethoxy)-2-methylpropiophenone (Irgacure 2959), camphorquinone (CAS-No: 10373-78-1), ethyl-dimethylamino benzoate (EDAB), diphenyliodonium hexafluorophosphate (DPIHFP). Water soluble photoinitiators active in the visible spectrum include but are not limited to Eosin Y, riboflavin, ruthenium(II) chloride hexahydrate [Ru(II)(bpy)₃]Cl₂, ruthenium sodium persulfate (Ru-SPS),

In certain particular embodiments, the photoinitiator is lithium phenyl-2,4,6-trimethylbenzoylphosphinate (LAP).

Photoinitiator concentrations may vary based on the photoinitiator types. A list of concentrations of initiators that have worked well in the inventors' hands include: LAP (0.1-2 mg/ml), Ru (0.1-10 mM)+SPS (0.1-10 mM), Irgacure 2959 (0.2-2 mg/ml).

### Refractive Index (RI) Matching Agent and Photo Absorptive Dye

Any RI matching agent in certain embodiments, the RI matching agent is selected from propylene glycol, poly(ethylene glycol) (PEG), poly(vinyl alcohol) (PVA), poly(lactic acid) (PLA), Bovine serum albumin (BSA), and iodixanol (CAS-No: 92339-11-2). In certain particular embodiments, the RI matching agent is iodixanol.

Any photo absorptive dye in certain embodiments, the photo absorptive dye is selected from Brilliant Blue FCF (CAS-No: 3844-45-9), Indigotine (CAS-No: 860-22-0), Fast Green FCF (CAS-No: 2353-45-9), Erythrosin (CAS-No: 16423-68-0), Tartrazine (CAS-No: 1934-21-0), Sunset Yellow FCF (CAS-No: 2783-94-0).

In certain particular embodiments, the photo absorptive dye is Sunset Yellow FCF.

The amount of refracting index matching that is conducive to improving the outcome of the method, depends on the refractive index of the components of the reaction. In one application, the refractive index may be matched to microbeads or cells that may be embedded in the matrix that is used for printing.

The refractive index (RI) matching agent slowly removed from the matrix by diffusion after the method has produced the implant of choice.

The choice of the amount of RI matching agent is based on the RI of the additive in the resin, which includes cells or other particles such as gelatin microbeads or silver micro- and nano-particles or conductive matrices. For instance, for cells, the RI is around 1.375, so one would need around 30% w/v of lodixanol (RI=1.384) in a GelNB/GelSH resin (original RI=1.34) to increase its RI to 1.375. This RI matching is essential if one needs to print with high cell densities, as RI matching can reduce the light scattering from the cells as cells essentially become “transparent” to the incoming light.

### Optical Characteristics

The inventors have employed wavelengths of 405 nm, 450 nm, 473 nm, 488 nm, 520 nm, 638 nm and 750 nm. Photoinitiators useful for practicing the invention are discussed in detail in Lee et al., Chem. Rev. 2020, 120, 19, 10950-1102.

Other ranges that have worked well in the inventors' hands include 360-375 nm, 400-420 nm, 450-475 nm, and 550-575 nm.

The spatially coherent light beams are characterized by a diameter of 1 to 100 μm.

In certain embodiments, the spatially coherent light beams are characterized by a diameter of 3 to 40 μm. In certain more particular embodiments, the spatially coherent light beams are characterized by a diameter of 5 μm to 15 μm.

In certain even more particular embodiments, the spatially coherent light beams are characterized by a diameter of 7.5 μm to 12.5 μm.

In general, the diameter of the micropillars is between 1 μm to 100 μm. In certain embodiments, the diameter of the micropillars is between 1 μm and 50 μm. In certain embodiments, the diameter of the micropillars is between 2 μm and 50 μm. In certain embodiments, the diameter of the micropillars is between 3 μm and 40 μm. In certain embodiments, the diameter of the micropillars is between 7.5 μm and 12.5 μm.

In certain embodiments, the mean diameter of the micropillars is between 1 μm to 100 μm.

In certain embodiments, the mean diameter of the micropillars is between 7.5 μm to 12.5 μm. In certain particular diameters, ≥75%, ≥80%, ≥85, ≥90% or even ≥90% of the micropillars are characterized by a diameter between 7.5 μm to 12.5 μm.

The optical set-up influences the performance of the system. The inventors employed a range of spatially coherent light beams/coherence lengths of 3.7 to 17.2 μm. In certain embodiments, this coherence length varies from 1 to 100 μm.

In certain embodiments, the composition is illuminated by a plurality of spatially coherent light beams comprising more than 100 light beams.

The macroscopic dimensions of the light beam may be varied by changing the projection image through a digital micromirror device or spatial light modulator.

In certain embodiments, the dimensions for a cross-section of the beam can be between 2×2 μm² (single pixel projection), around 2×2 cm² right after the light shaping due to the DMD, and may even reach 10×10 cm² (upon expansion of the light beam due to telescopic lenses).

### Speckle Pattern Generation and Modulation

The speckle pattern is a characteristic quality of the laser light source. The term speckle pattern refers to the granular pattern that is observed when laser light interacts with an optical medium, inducing the light waves to interfere with each other constructively or destructively, creating areas of bright and dark spots. These bright and dark spots form a random pattern, the speckle pattern.

The exact speckle pattern observed depends on various factors, including the laser's coherence length, homogenization of laser light, the roughness of the surface, the magnification of the projected image after laser shaping, the size of the laser beam, and the distance between the surface and the observer. If any of these factors change, the speckle pattern will also change.

In certain embodiments, homogenization of laser light can be achieved using a Fly's eye homogenizer lens (a lens that transforms a round beam with a Gaussian profile into a homogenous illuminated rectangular area) to remove the gaussian distribution of light intensity found in the lasers. This is important to achieve a substantially uniform light intensity throughout the projected light after the shaping. Homogenization does not remove the individual spackle patterns, but rather just homogenizes the light intensity. Importantly, this is an optional step, as a FLight projection apparatus would still work without the homogenization, but homogenization may lead to better print resolution and also more uniformly crosslinked constructs as the light intensity is uniform throughout the projected image.

Upon a change of the speckle patterns, the diameters of the microfilament/micropillar and the associated microchannels/microvoids also change. This may present different effects on the cells (e.g. genetic changes due to nuclear confinement of the cells in-between the microfilaments/micropillars) and also cause change in nutrient diffusion characteristics.

### Digital Micromirror Device/Spatial Light Modulator

A Digital Micromirror Device (DMD) is an optical semiconductor chip consisting of an array of microscopic mirrors that can be individually controlled to reflect or redirect light. It is widely used in display technology, projection systems, and optical communications. Spatial Light Modulators (SLMs) are devices that modify the phase, intensity, or polarization of light, enabling complex light shaping for applications like holography, optical trapping, adaptive optics, and beam steering.

Overall, the utility of applying DMD or SLM will be to shape the incoming light into a final projected light that goes into the photo-responsive resin. They are both important to get the final desired light projection shape.

DMD or SLM can be commercially sourced. In the particular case, DMD was from Texas instruments and the SLM was from Thorlabs. Meshes can be introduced into the light beam, leading to microfeatures in the dimension between 30 and 150 μm being generated.

Based on the ratio of microbeams and voids, the material shown in the examples is characterized in that about 50% of space is filled by beam material, thus, one cubic centimeter (cm³) of implant or hydrogel constructs contains approximately 6370 microbeams, which are of cylindrical shape. For the theoretical calculation, the microbeams are considered as cylinders.

In certain embodiments, the composition is illuminated by a first plurality of spatially coherent light beams aligned in a first direction.

In certain particular embodiments, the first plurality of spatially coherent light beams forms a first pattern.

### Sequential Build-Up of Further Structures

In certain embodiments, the composition is illuminated, in a subsequent or parallel second illumination step, by a second plurality of spatially coherent light beams aligned in a second direction.

In certain embodiments, the second plurality of spatially coherent light beams forms a second pattern.

In particular embodiments, the first direction and the second direction are parallel.

The first direction and the second direction can be, according to some embodiments, be arranged at an angle of 1° to 180°. In certain particular embodiments, the angle ranges from 15° to 180°.

In certain embodiments, the first and/or second pattern are arranged in cylindrical form i.e. in the form of a beam, in form of a filled solid cylinder or in the form of a hollow cylinder, in form of a sheet, or as parallel strands between anchoring tendon-like support sheets similar to the geometry of a muscle fiber arrangement (see FIG. 19). The projections can also be made to vary along different layers of the construct so as to create more complex geometries comprising microbeams.

In principle, any arbitrary cross-section is possible.

In certain embodiments, subsequent to said first and optionally, second illumination step, a second composition is added to the container, said second composition comprising
a second polymer susceptible to photo-crosslinking, particularly thiol-ene mediated photo-crosslinking,
a photoinitiator and
optionally, a refractive index matching agent (e.g., iodixanol up to 80% w/v) which can increase or decrease the refractive index of photoresin formula
optionally, photo absorptive dye (e.g., Sunset Yellow or FCF at concentration up to 500 μg/mL)
optionally, a crosslinking agent.

In a third illumination step, the composition is illuminated with a third plurality of spatially coherent light beams. This allows building a second structure complementing the first structure, by a different or the same biopolymer.

This can be repeated again to build up yet another structure where useful. The number of steps is not limited by principle, the method can proceed to infinite numbers of LBP steps. Any particular projection process can be repeated infinitely to create hydrogel structures with infinite length on the projection axis. Alternatively, the entire projection process can be split into sub-steps by increasing the number of projection steps/decreasing the projection dose of each step to achieve a better projection quality/resolution.

In certain embodiments, any first composition not photo-crosslinked is drained from the container and the container is filled with said second composition.

It is not necessarily required to drain the contents out of the container, the first composition can can simply remain while new material is added.

In certain embodiments, in the first, second, third or any subsequent illumination step, illumination with a plurality of spatially coherent light beams is effected from a bottom side of the container onto a substrate, leading to formation of a plurality of polymer microbeams; the substrate is moved upwards away from the bottom of the container under continuous illumination, thereby forming a plurality of microbeams protruding from the substrate. This allows building very long structures and thus facilitates the generation of tendon-supporting implants, nerve guides and other structures exceeding the dimensions limited by the length of the beam. Tendon-like support structures are depicted, inter alia, in Nourissat et al., Nature Reviews Rheumatology 11, 223-233 (2015).

In certain embodiments, the spatially coherent light beam is characterized by one or more of the following parameters:
a wavelength of 360 to 800 nm,
a coherence length of 1 to 100 μm (particularly 3 to 20 μm);
a light dose of 10 to 5000 mJ/cm², particularly of 90 to 200 mJ/cm²,
a duration of 0.1 s to 100 s, particularly from 1 s to 4 s;
an energy density of 1 to 1000 mW/cm², particularly of 1-500 mW/cm², more particularly of 50 to 60 mW/cm².

The values for coherence length given here in minimum and maximum were measured from different microbeams created using different materials.

The light dose also depends on the length of the hydrogel structure (i.e., the depth of light penetration). For example, a depth of 6 mm requires a light dose of 110 mJ/cm², and 195 mJ/cm² corresponds to a depth of 15 mm. 50 to 60 mW/cm² corresponds to the light intensity measured before the photoresin container, i.e. the light intensity before entering, and reaching the photoresin.

A light dose of 70 to 250 mJ/cm² incorporated to a projection of about 1 to 4 seconds. (100% grayscale)

### Hydrogels Prepared by the Method of the Invention

Another aspect of the invention relates to a three-dimensional hydrogel implant comprising or essentially consisting of a first plurality of microbeams, also referred to as microfilaments herein, of a first photo-crosslinked polymer, said three-dimensional hydrogel implant obtained by a method according to the method of the invention as laid out in any of its embodiments in the present specification.

In certain embodiments, the three-dimensional hydrogel bioimplant according to the invention comprises a second plurality of microbeams of the first photo-crosslinked polymer.

In certain embodiments, the three-dimensional hydrogel bioimplant comprises a third plurality of microbeams of a second photo-crosslinked polymer.

In certain embodiments, said first, second and/or third plurality of microbeams is characterized by a diameter of each of the microbeams of said first, second and/or third plurality of microbeams ranging from 1 μm to 50 μm. In particular embodiments, the diameter ranges from 2 to 30 μm. These dimensions allow the microstructures to act as excellent cell-guidance cues, leading an aligned cell morphology and extracellular matrix deposition, which is essential to creating a biomimetic anisotropic tissue.

In certain embodiments, said first, second and/or third plurality of microbeams is characterized by >75% (particularly >80%, ≥85%, ≥90%, ≥95% or even ≥98%) of said microbeams of said first, second and/or third plurality of microbeams having an alignment of ≤2° deviation from a longitudinal axis common to each of the first, second or third plurality of microbeams (one axis for each plurality).

In certain embodiments, said first, second and/or third plurality of microbeams is characterized by >75% (particularly >80%, ≥85%, ≥90%, ≥95% or even ≥98%) of said microbeams of said first, second and/or third plurality having a length of 100 μm to 2 cm, particularly having a length of 100 μm to 5 cm.

The microbeams may, in some cases, fuse with other microbeams which may affect their discernability. In certain embodiments, a 2 cm constructs, the length of individual microbeams could be anywhere between 100 μm to 2 cm, making up a coherent plurality of microbeams.

The target length of the structure depends on the tissue structures of interest to the application. Anatomically, the most muscle tissue in vivo is more than 5 cm in length. There is no upper limit to the length of the microbeams fabricated by the method of the invention, which has the potential to create structures up to the largest primary muscle groups in the leg, more than 27 cm long.

In certain embodiments, the three-dimensional hydrogel bioimplant according to the invention is characterized by channel structures having a diameter of 100 μm to 1 mm, particularly by a diameter of 400 μm to 600 μm. Larger structures are of course possible; the upper limit of the diameter depends only on the size of the photoresin container. Channel structure dimensions may be determined by taking confocal images of fluorescently labeled materials (the materials can be labeled with Rhodamine or FITC) and measuring the microbeam/microfilament/microchannel diameters in ImageJ length measurement tool by manually measuring the diameter of each microfilament.

In certain embodiments, the hydrogel implant encompasses cells. Cells that lend themselves to being engrafted in the implants according to the invention particularly include stem cells (adipose derived, mesenchymal, induced pluripotent, embryonic) and differentiated cells (myoblasts, fibroblasts, neurons, tenocytes, macrophages, chondrocytes, osteoblasts). The cells will be encapsulated in the grafts or post-seeded over the grafts.

In certain embodiments, the three-dimensional hydrogel bioimplant according to the invention may comprise a growth factor, or a combination of several growth factors. In particular embodiments, growth factors may be selected from as nerve growth factor, neurotropic growth factor, vascular endothelial growth factor (VEGF), transforming growth factor (TGF-β), etc.), all of which have been demonstrated to promote tissue regeneration.

Also encompassed in the invention is developed a Filamented Light (FLight) projection system which allows concomitant and continuous resin feeding and filamented light projection, leading to long anisotropic constructs with cell-guiding filaments present across the entire length of the constructs.

The system according to the invention uses spatially coherent light from a high intensity laser or a Standard Kohler illumination system in a particular FLight projection setup. The Standard Kohler illumination system is just one of the embodiments which uses an LED as the light source. This sort of illumination system introduces speckles in the LED light profile. Alternatively, and advantageously, a system for generating the source light for practicing the invention employs a laser light, as laser intrinsically feature a speckled distribution of the light beam. This laser light could be expanded through telescopic lenses, and then shaped through a digital micromirror device to obtain the desired beam projection image. This image could then be further expanded if needed through another set of telescopic lenses, and finally projected onto the photoresin container (see FIG. 24).

The projected light features static noise patterns of the intensity, an intrinsic property of the laser, which is then projected onto a photoresin-filled cuvette. The resin crosslinks first where the intensity is higher, which causes a localized increase in the refractive index. This induces self-focusing of the light solely along the crosslinked resin, which further causes filamentation of the light beam into individual microfilaments along the length of the resin. Finally, the resin crosslinks along these microfilaments, resulting in a porous anisotropic construct. The complete process of gradual filamentation of the light into several microbeams within a photo-responsive matrix is called optical modulation instability (OMI). The process is compatible with both step-growth and chain-growth polymerization commonly used in photo-crosslinkable hydrogel systems.

In order to increase compatibility with processes where long constructs need to be fabricated, the inventors have introduced a continuous feeding and FLight projection mechanism in a particular exemplary embodiment of the FLight system (FIG. 23; light path is illustrated in FIG. 24). As opposed to multiple projections stacked one on top of another, the new projection scheme uses continuous resin feeding within a cuvette and FLight projection from the top, which allows for the fabrication of long constructs with microfilaments running through the entire length of the constructs (FIG. 25). Where necessary, over-exposure of the resin due to the light coming from the top may be prevented through the use of a photo-absorber (FIG. 26), which absorbs the light penetrating into the polymer network and also ensures a high resolution of printing by quenching the light intensity in off-target regions (FIG. 27). The presence of photo-absorber also does not have any effect on the presence of microfilaments (FIG. 26).

In addition to continuous microfilaments within the long cm-scale constructs, the invention also provides another setup where the cuvette is attached onto a translation and rotation stage. By changing moving and rotating the cuvettes while controlling the FLight projections from the top or sides (FIG. 29 and FIG. 30), this setup provides the ability to generate a broad range of microfilament orientations (helical, crisscross) within the constructs. Furthermore, a pneumatic resin feeding mechanisms can be used to feed multiple types of resins into the cuvette while the FLight projections are ongoing, which can lead to the formation of multi-material tissue interface models (such as a myotendinous junction).

The variation of the aperture diameter in the standard Kohler illumination setup can vary the size of the microfilaments, for example between 5-30 μm. A similar effect can also be achieved with a laser-based illumination system, where the telescopic lens apparatus (originally used to collimate the light beam) can be used to expand/contract the laser speckle pattern by changing the focal length or the distances between the lenses used therein (FIG. 31). Pixelization of the images (FIG. 32) being projected through the digital micromirror device (DMD) or spatial light modulator can be used to further increase the porosity of the fabricated anisotropic constructs, which can help with nutrient transport and cell guidance.

Meshes or gratings placed along the light path before, between or after the collimating lenses (illustrated in FIG. 33), can be used to further introduce microporosities within the constructs (FIG. 33 B, C and FIG. 34). Furthermore, through the use of two or more gratings featuring different sizes and different relative orientations w.r.t. the light path (FIG. 35), Moire patterns can be introduced within the light path, which also allows the creation of unique microarchitectures (FIG. 36).

To summarized, key aspects and advantages of the FLight technology as presented herein include, without being limited to:
Continuous FLight projection and resin feeding can fabricate long constructs up to 20 cm in length, which caters to the scale of most anisotropic human tissues.
Changing distance between telescopic lenses, or by using lenses with different focal lengths, in the FLight projection setup can alter speckle patterns, which can change the microfilament diameter. This can allow control over the cellular microenvironment for different tissues.
Changing distance between telescopic lenses, or by using lenses with different focal lengths, in the FLight projection setup can increase or decrease the sizes of the images being projected, which could allow small or large-scale tissue fabrication.
A biocompatible Photoabsorber (e.g., FD&C Yellow) and free-radical inhibitor (e.g., TEMPO) can be added to the photoresin to control light penetration depth within the photoresin, leading to a uniformly crosslinked construct with continuous FLight projections.
High/low frequency of on/off FLight projection can lead to a better print resolution within FLight biofabrication.
The invention provides a variety of methodologies to introduce microstructure within the constructs, leading to better tissue maturation and regenerative effects.
The invention provides a variety of systems, with or without a digital micromirror device (DMD). If no DMD is present, it is possible to add inserts along the light path to change the projection image and the speckle patterns. For example, with metal meshes (single or multiple) along the light path which cause different micropatterns within the crosslinked constructs. Alternatively, a DMD may be present to control the projection image, where the image pixelization can create porous microstructures within the constructs.
Multi-direction projection is possible by switching mirrors, leading to a higher degree of freedom in the orientation of the microfilaments.

It is possible to change the position of the printing cuvette and rotating the cuvette concomitantly in order to create complex filament orientations within the constructs. This may be achieved for example by FLight projections from the top, bottom and the sides, or by changing the material constitution and feeding rates, or by changing the light intensity to alter the crosslinking rates.

can be used

Bottom-up projection can also be used instead of top-down, with the substrate continuously moving upwards.

Another aspect is that the invention facilitates continuous printing based on oxygen diffusion (i.e. concept similar to the Continuous liquid interface production (CLIP) printing, but with the resulting constructs featuring continuous microfilaments. This is only possible with limited photo-crosslinking strategies as provided herein.

The 3D shape of the constructs generated by the methods according to the invention may be changed by:
a. changing the projection images from a DMD while the continuous FLight projection method is being executed.
b. changing the projection image though interference patterns caused due to light passing through multiple wire meshes (which act as diffraction gratings). This procedure would not require a DMD.
c. changing the projection image though blocking the path of light through a stencil or mask or a wire mesh. This procedure would not require a DMD.

The invention further encompasses the following items:
Item 1. A method for making a three-dimensional hydrogel bioimplant comprising:
   a. providing a first composition susceptible to photo-crosslinking in a container, said first composition comprising:
      i. a first polymer susceptible to photo-crosslinking, particularly to thiol-ene crosslinking, but also susceptible to free radical polymerization chain-growth crosslinking;
      ii. a photoinitiator and
      iii. optionally, a refractive index matching agent (e.g., iodixanol up to 80% w/v) which can increase or decrease the refractive index of photoresin formula
      iv. optionally, photo absorptive dye (e.g., Sunset Yellow or FCF at concentration up to 500 μg/mL)
      v. optionally, a crosslinking agent and
   b. in a first illumination step, illuminating the composition with a plurality of spatially coherent light beams,
      thereby generating a plurality of micropillars in the composition.
Item 2. The method according to item 1, wherein said spatially coherent light beams are characterized by a diameter of 1 to 100 μm, particularly of 3 to 40 μm, even more particularly from 5 μm to 15 μm;
   most particularly by a diameter of 7.5 μm to 12.5 μm.
Item 3. The method according to item 1 or 2, wherein the composition is illuminated by more than 100 light beams.
Item 4. The method according to any one of the preceding items, wherein the composition is illuminated by a first plurality of spatially coherent light beams aligned in a first direction;
   particularly wherein said first plurality of spatially coherent light beams forms a first pattern.
Item 5. The method according to item 4, wherein the composition is illuminated, in a second illumination step, by a second plurality of spatially coherent light beams aligned in a second direction;
   particularly wherein said second plurality of spatially coherent light beams forms a second pattern.
Item 6. The method according to item 5, wherein said first direction and said second direction are parallel.
Item 7. The method according to item 5, wherein said first direction and said second direction are arranged at an angle of 10 to 180°, particularly 150 to 180°.
Item 8. The method according to any one of the preceding items 4 to 7, wherein said first and/or said second pattern are arranged in cylindrical form, in form of a sheet, or as parallel strands between anchoring tendon-like support sheets.
Item 9. The method according to any one of the preceding items, wherein subsequent to said first illumination step, a second composition is added to the container, said second composition comprising
   i. a second polymer susceptible to photo-crosslinking,
   ii. a photoinitiator and
   iii. optionally, a refractive index matching agent (e.g., iodixanol up to 80% w/v) which can increase or decrease the refractive index of photoresin formula
   iv. optionally, photo absorptive dye (e.g., Sunset Yellow or FCF yellow at concentration up to 500 μg/mL)
   v. optionally, a crosslinking agent and
   in a third illumination step, illuminating the composition with a third plurality of spatially coherent light beams.
Item 10. The method of item 9, wherein any first composition not photo-crosslinked is drained from the container and the container is filled with said second composition.
Item 11. The method according to any one of the preceding items, wherein in the first, second, third or any subsequent illumination step, illumination is effected from a bottom side of the container onto a substrate; the substrate is moved upwards away from the bottom of the container under continuous illumination, thereby forming a plurality of microbeams protruding from the substrate, particularly wherein microbeams are prevalent through the length of the hydrogel constructs in a longitudinal axis.
Item 12. The method according to any one of the preceding items, wherein the polymer susceptible to photo-crosslinking is a biopolymer functionalized by covalent attachment of carbon-carbon double bond (ene) containing moieties or a methacrylate-functionalized biopolymer.
Item 13. The method according to item 12, wherein the biopolymer is selected from the group comprised of gelatin, hyaluronan, alginate, collagen, chitosan, silk fibroin, cellulose.
Item 14. The method according to item 12 or 13, wherein the carbon-carbon double bond (ene) containing moieties are selected from the group comprised of a norbornene carboxylic acid or dicarboxylic acid, methacrylic acid ester or -amide, acrylic acid ester or -amide, and vinyl esters.
Item 15. The method according to any one of the preceding items, wherein the polymer susceptible to photo-crosslinking is selected from the group comprised of norbornene-functionalized gelatin, norbornene-functionalized collagen, norbornene-functionalized chitosan, norbornene-functionalized hyaluronan, gelatin methacryloyl (Gel-MA), hyaluronic acid methacryloyl (HA-MA), alginate methacryloyl (Alg-MA);
   particularly wherein the polymer susceptible to thiol-ene photo-crosslinking is norbornene-functionalized gelatin.
Item 16. The method according to item 15, wherein the norbornene-functionalized gelatin is characterized by a degree of substitution of 10% to 90%, particularly from 47% to 50%, the methacrylate-functionalized gelatin is characterized by a degree of 10% to 90%, particularly from 45-60%.
Item 17. The method according to any one of the preceding items, wherein the crosslinking agent is a thiol-functionalized poly(ethyleneglycol), particularly a pentaerythrol-PEG-thiol [C(CH₂O(CH₂CH₂O)ₙCH₂CH₂SH)₄]
Item 18. The method according to any one of the previous items, wherein the photoinitiator is selected from the group comprised of lithium phenyl-2,4,6-trimethylbenzoylphosphinate (LAP), 2,4,6-trimethylbenzoyldiphenylphosphine oxide (TPO), bis-acylphosphine oxide (BAPO), 2-hydroxy-4′-(2-hydroxyethoxy)-2-methylpropiophenone (Irgacure 2959), camphorquinone (CAS-No: 10373-78-1), ethyl-dimethylamino benzoate (EDAB), diphenyliodonium hexafluorophosphate (DPIHFP),
   particularly wherein the photoinitiator is LAP.
Item 19. The method according to any one of the preceding items, wherein the refractive index matching agent is selected from Propylene glycol, Poly(ethylene glycol) (PEG), Poly(vinyl alcohol) (PVA), poly(lactic acid) (PLA), Bovine serum albumin (BSA), and iodixanol (CAS-No: 92339-11-2), particularly wherein the refractive index matching agent is iodixanol.
Item 20. The method according to any one of the previous items, wherein the photo absorptive dye is selected from Brilliant Blue FCF (CAS-No: 3844-45-9), Indigotine (CAS-No: 860-22-0), Fast Green FCF (CAS-No: 2353-45-9), Erythrosin (CAS-No: 16423-68-0), Tartrazine (CAS-No: 1934-21-0), Sunset Yellow FCF (CAS-No: 2783-94-0), Particularly wherein the photo absorptive dye is Sunset Yellow FCF.
Item 21. The method according to any one of the preceding items, wherein the spatially coherent light beam is characterized by
   a. a wavelength of 360 to 800 nm;
   b. a coherence length of 1 to 100 μm, particularly of 3 to 20 μm;
   c. a light dose of 10 to 5000 mJ/cm², particularly of 90 to 200 mJ/cm²,
   d. a duration of 0.1 s to 100 s, particularly from 1 s to 4 s;
   e. an energy density of 1 to 500 mW/cm², particularly of 50 to 60 mW/cm².
Item 22. A three-dimensional hydrogel implant comprising or essentially consisting of a first plurality of microbeams of a first photo-crosslinked polymer, said three-dimensional hydrogel implant obtained by a method according to any one of the previous items.
Item 23. The three-dimensional hydrogel bioimplant according to item 22, comprising a second plurality of microbeams of the first photo-crosslinked polymer.
Item 24. The three-dimensional hydrogel bioimplant according to item 22 or 23, comprising a third plurality of microbeams of a second photo-crosslinked polymer.
Item 25. The three-dimensional hydrogel bioimplant according to any one of the items 22 to 24, wherein said first, second, third and/or any subsequent plurality of microbeams is characterized by
   a. each of the microbeams having a diameter ranging from 1 μm to 50 μm;
   b. >75% (particularly >80%, ≥85%, ≥90%, ≥95% or even ≥98%) of said microbeams of said plurality having an alignment of ≤2° deviation from a longitudinal axis, and/or
   c. >75% (particularly >80%, ≥85%, ≥90%, ≥95% or even ≥98%) of said microbeams of said plurality having a length of >2 cm, particularly >5 cm.
   d. Microbeams prevalent through the length of the hydrogel constructs in a longitudinal axis.
Item 26. The three-dimensional hydrogel bioimplant according to any one of the items 22 to 25, characterized by channel structures having a diameter of 100 μm to 10 mm, particularly by a diameter of 200 μm to 1000 μm; more particularly by a diameter of 400 μm to 600 μm.

Wherever alternatives for single separable features are laid out herein as “embodiments”, it is to be understood that such alternatives may be combined freely to form discrete embodiments of the invention disclosed herein.

The invention is further illustrated by the following examples and figures, from which further embodiments and advantages can be drawn. These examples are meant to illustrate the invention but not to limit its scope.

### DESCRIPTION OF THE FIGURES

FIG. 1 a) shows Light Beam Projection (LBP)—biofabrication strategy of anisotropic constructs containing beam-like microstructures with efficient cell guidance properties. The figure shows a schematic of LBP strategy for aligned microbeams and their cell guidance properties. b) long and continuous microbeams and microchannels are generated in anisotropic hydrogel constructs that correspond to the local maximum and minimum of light intensity.

FIG. 2 shows the characterization of hydrogel construct containing microbeams. a) Bright-field image of entire 1 mm diameter and 7 mm long hydrogel sample created using 2.88% w/v Gel-NB/4PEG-SH photoresin. Scale bar is 200 μm. b) Magnified image of microbeam structuring of the hydrogel sample. Scale bar is 20 μm. c) Distribution of the microbeams diameter, as well as the distribution of angles between microbeam orientation and direction of projection (all data was collected from BF images). d) Bright-field images of microbeams in hydrogel samples fabricated using 5% w/v Gel-NB/4PEG-SH, 5% w/v Gel-MA, 2% w/v HA-MA, 2% w/v Alg-MA photoresin. scale bar is 50 μm. e) Diameter of microbeams under different spatial coherence lengths by adjusting the aperture diameter (AD) using a standard Köhler's illumination system. f) Left panel: tensile mechanical properties of hydrogel constructs containing microbeam fabricated using different concentrations of Gel-NB/4PEG-SH photoresin. Right panel: tensile tests of bulk hydrogel and microbeam-contained hydrogel samples created using 2.88% w/v Gel-NB/4PEG-SH or 5% w/v Gel-MA photoresin. LBP and bulk hydrogel samples were produced as hydrogel strands 12 mm long and 1 mm in diameter for tensile testing. g) Left panel: compressive moduli calculated from the stress-strain curves obtained from hydrogel samples prepared using 1.44% w/v, 2.88% w/v, and 5% w/v of Gel-NB/4PEG-SH photoresin. Right panel: elastic modulus of bulk hydrogel samples and microbeam-contained hydrogel samples fabricated using 2.88% w/v Gel-NB/4PEG-SH or 5% w/v Gel-MA photoresin, n=3. Hydrogel samples for compressive testing were prepared as cylinders with a diameter of 5 mm and a height of 4 mm. h) Cycle loading-unloading tensile curves of hydrogel sample fabricated using 2.88% w/v Gel-NB/4PEG-SH photoresin (test performed in air condition, target strain is 50%).

FIG. 3 shows microchannels present in the LBP hydrogel matrix support cell migration. a) Projected image of microbeams in hydrogel matrix produced by LBP using 5% w/v fluorescent-labelled Gel-MA photoresin. Arrows indicate the microchannels between microbeams after washing. Scale bar is 20 μm. b) Percentage of microchannels in 3D hydrogel volume created with varying spatial coherence length. All data were measured from 3D confocal fluorescent images using Imaris software. c) The ratio of void area in each scan step on Z-stack of confocal fluorescent images (data collected using Image J, 120 steps in Z-direction, 0.5 μm distance between steps). d) Fluorescent images of cell-laden hydrogel matrix. Nuclei and NHDFs were stained by Hoechst 33342 and Calcein-AM after 1 hour, 1, 3 and 7 days of culture. At each time point, the bottom left panels are projected images (max intensity) and the bottom-right panels are single, sliced images. Arrows indicate the original positions of fibroblasts after projection and migration into the microchannels. Scale bars are 20 μm. e) Mean diameter of microchannels in hydrogel constructs produced by light beams with different spatial coherence lengths using 5% w/v fluorescent-labeled Gel-MA photoresin. The diameter was measured as the distance between microbeams in fluorescent images. The microchannel was randomly selected from 8 different z-step of confocal images (10 data per z-step). f) Mean aspect ratio of microchannels in hydrogel constructs fabricated with varying coherence lengths of the light beam. g) Cell aspect ratio at different time points. All data were collected from fluorescent images (n=3).

FIG. 4 shows microchannels provide mechanical confinement to increase proliferation and collagen formation. Immunofluorescence staining of ki67 in hydrogel samples fabricated from different strategies was performed after 1 hour, 1, 3 and 7 days of culture. a) Ratio of Ki67 positive cell in hydrogel samples at different time points. All data were calculated using Image J (n=3). b) Relative change of gene expression in cell-laden/cell-seeded hydrogel constructs after 1 hour, 1 day, 3 days, and 7 days of culture. The fold change given in the heat map is the mean value. All data were collected from 3 biological replicates of hydrogel samples (with 2 technical replicates per sample).

FIG. 5 shows cell guidance properties of microbeams and maturation of highly aligned tissue constructs. Evidence of NHDFs alignment was obtained using phalloidin staining, and immunofluorescence staining of highly aligned collagen type I in LBP hydrogels after 2 weeks of culture. Alignment of HTs and collagen type I in LBP hydrogel constructs was visualized after 2 weeks of culture. Immunofluorescent images were obtained as evidence of highly aligned vasculature in a NHDFs-HUVEC co-culture system after 2 weeks of incubation. Continuous blood capillaries with length up to 1 mm were observed. Immunofluorescence staining of Myosin Heavy Chain (MyHC) and collagen type I was performed in muscle tissue constructs after 3 weeks of culture in differentiation medium. a) Distribution of NHDFs orientation in LBP hydrogels and in bulk hydrogels after 2 weeks of culture. All data were collected from fluorescent images (n=3). b) Ratio of collagen type I positive area in NHDFs-laden LBP hydrogels and in NHDFs-laden bulk hydrogels (n=3). c) Mean value of NHDFs' nuclei aspect ratio in microbeam-contained and bulk hydrogel samples (n=3). d) Distribution of myotubes' orientation in tissue constructs after 3 weeks of culture in differentiation medium. All data were calculated from the fluorescent images using ImageJ (n=3). e) Fusion index of myoblasts and f) ratio of nuclei number per fused myotubes to evaluate muscle maturation. All results were measured from fluorescence images (n=3).

FIG. 6 shows advanced light beam projection for biofabrication of complex hydrogel constructs and potential for multi-materials LBP. a) Top panels: the photograph and bright-field image of cell-laden tubular hydrogel constructs fabricated using 2.88% w/v Gel-NB/4PEG-SH bioresin. b) Middle and bottom panels: the fluorescent images of tubular constructs from front view and side view. The encapsulated NHDFs were labeled by Calcein-AM and the RhoD-labeled Dextran was added into tubular hydrogel constructs after 7 days of culture. Scale bars are 100 μm. c) Schematic illustration of two-step LBP for heterogeneous biofabrication of anisotropic tissues. d) Photographs and full scanned fluorescent image of varying hydrogel constructs achieved by two-step LBP using 5% w/v FITC- and Rhod-labeled Gel-MA photoresin. Scale bar are 2 mm in the photograph, and 1 mm in the fluorescent image. e) Fluorescent images of hydrogel constructs fabricated by two-step LBP on day 0 and 7. The NHDFs were labeled by Cell tracker Green or Red, and encapsulated in the hydrogel constructs fabricated using 5% and 2.88% w/v Gel-NB/4PEG-SH photoresin, respectively. The side view of cell alignment after 7 days of culture were generated by Imaris from a full scanned 3D fluorescent image. Scale bars are 200 μm in all fluorescent images.

FIG. 7 shows a) Distribution of the microbeam angle between projection direction and b) distribution of microbeam diameter in hydrogel samples fabricated using different photoresin. All data was collected from BF images.

FIG. 8 shows the diameter of microbeams is tunable by adjusting the spatial coherence length independently from the projection area. a) The tunable spatial coherence length of light beams by adjusting the aperture diameter in assembled Köhler's illumination system (with 405 nm LED). b) The diameter of hydrogel samples at different field diameters. c) The mean diameter of microbeams fabricated at different field diameters.

FIG. 9 shows characterization of porosity in hydrogel samples containing microbeams. The ratio of void in each slice of fluorescent images, measured from hydrogel samples fabricated by standard Köhler's illumination system under different aperture diameters using 5% w/v Rhod-labelled Gel-MA Photoresin. Data was collected using Image J, 120 steps, step size of 0.5 μm.

FIG. 10 shows characterization of cell migration across microchannels. a) Fluorescent images of cell migration on a bulk hydrogel surface or hydrogel containing microbeams after 1 day, 3 days and 7 days of culture. Hydrogel constructs were fabricated using Rhod-labeled Gel-MA photoresin in both conditions (Red), and post-seeded fibroblasts were imaged using Calcein-AM (Green) and Hoechst 33342 (Cyan). Scale bars are 20 μm. b) The migration depth from the surface of hydrogel constructs at different time points. Data was measured using ImageJ from the above images.

FIG. 11 shows relative change of gene expressions in cell-laden/cell-seeded hydrogel constructs after 1 hour, 1 day, 3 days, and 7 days of culture as depicted in FIG. 5.

FIG. 12 shows cell viability in hydrogel samples fabricated by different strategies. Live/Dead staining of fibroblasts embedded in hydrogel after 7 days of culture was performed using Calcein-AM and PI. Hollow indicated the hydrogel sample was fabricated using a projection image containing multiple pores with a diameter of 8 pixels (about 216 μm). Scale bars are 100 μm. The graphs give viability of varying types of cells in hydrogel samples fabricated from different strategies.

FIG. 13 shows collagen deposition in hydrogel construct containing microbeams. a) Negative control of collagen type I staining on a cell-free hydrogel sample after 2 weeks incubation in PBS. b-d) The immunofluorescent staining of deposited collagen fiber in hydrogel sample containing microbeams after 2 weeks of culture. Microbeams were fabricated using 5% w/v Rhod-labeled Gel-MA, scale bar is 20 μm.

FIG. 14 shows cross-section view of aligned blood capillary in hydrogel construct after 2 weeks of co-culture. The yellow arrows in the x-z and y-z view of fluorescent images indicated the typical lumen structures formed in the aligned capillary. Scale bars are 20 μm.

FIG. 15 shows optimization of light dose for projection of hydrogel sample containing hollow structures. a) Projection image used in optimization of light dose (top) and photograph of projected hydrogel constructs (bottom). The projection image consists of a series of the square blocks with different gray levels (50-100%) and contains different shaped hollow structures with dimension ranging from 1 to 6 pixels. Scale bar is 2 mm. b) Bright-field images of projected blocks. Scale bar is 500 μm. c) plots of intensity on the dash lines depicted in b. The black arrows indicated that the hollow structures in block hydrogel were created with varying diameters under 80% of the light dose. Defects within projected hydrogel constructs were found under 60% of the light dose. Loss of hollow features in hydrogel block under 100% of light dose demonstrated the over-crosslinking of hydrogel constructs.

FIG. 16 shows possible patterns for microbeam arrangements in implants generated by the invention.

FIG. 17 shows the schematic illustration of multi-direction projection. The first projection is performed to fabricate hydrogel structure container highly aligned microbeams, but from a single direction. Next, the container is moved to a specific angle (or the changing of the direction of the light source by applying mirror devices), and then a second projection is performed.

FIG. 18 shows bottom-up fabrication of long tissue constructs with continuous microbeams structures—bottom-up strategy can be used to create long tissue structures. By continuously adding photoresin (automated feeding) and tuning the projection images, it is possible to fabricate complex structures. In addition, by feeding different photoresin, multi z-step projection could be achieved. This allows the fabrication of more complex anisotropic tissue structures, such as muscle-tendon and tendon-bone interfaces.

FIG. 19 shows top-bottom fabrication of long tissue constructs with continuous microbeams structures—top-bottom strategy can be used to create long tissue structures. By continuously adding photoresin (automated feeding) and tuning the projection images, it is possible to fabricate complex structures, like with DLP. In addition, by feeding different photoresin, multi z-step projection could be achieved. This allows the fabrication of more complex anisotropic tissue structures, such as muscle-tendon and tendon-bone interfaces.

FIG. 20 shows hybrid projection strategies to fabricate tissue constructs with multi-direction microbeams. A second light system is produced horizontal to the first set. This hybrid system allows for the fabrication of structures containing microbeams in multiple directions. This will pave the way for the biofabrication of other tissue structures, such as cartilage tissue with multilayer structures. The subsequent projection from the top will allow the hydrogel structure to contain microwave beams in more than three directions, i.e. x-y-z axis.

FIG. 21 shows optical setup for multi-materials LBP. By setting triple light sources system (e.g., different wavelengths, 405 nm/625 nm), the photoresins are crosslinked to form the construct with microbeams, by adding various photoinitiators. This makes the LBP offers wider support of varying types of materials/tissues.

FIG. 22 shows the length of hydrogel constructs from one-time projection using 3% w/v Gel-NB based photo resin with different concentrations of photo absorptive dye (e.g., food dye: Sunset Yellow FCF). The results indicate the penetration depth of the light can be controlled in a certain range by adjusting the concentration of photo absorptive dye, which allows the avoidance of additional light does to the previous projection layers in multi-step projection, leading to an inhomogeneous crosslinking on the different layer of the hydrogel constructs.

FIG. 23 Schematic of FLight3D™ device with capabilities for side and front projections, and a syringe pump to continuously feed the resin into a cuvette while the light projections are executed from the top.

FIG. 24 Schematic illustrating the light path inside the enclosure for the optical components (shown in FIG. 23). The projections can be executed with or without a DMD to control the cross-section of the projected image. Within the cuvette, which is located outside of the enclosure for optical components, the light projection can be executed sideways (if the resin is already filled within the cuvette) or top-down depending (if the resin is being fed via the syringe pump).

FIG. 25 A. The length of the construct is limited by the light penetration of a single-step projection, while the fabrication of long constructs (>3 cm) can be achieved by multi-step projection. Still, inhomogeneous crosslinking between layers and over-crosslinking of the bottom layer is observed. By continuous FLight process, long constructs consisting of microfilaments throughout the entire length of constructs could be rapidly created. B. Elastic modulus in different areas of printed hydrogel constructs by performing compressive tests. For the long constructs created by multi-step projection, the difference in the elastic modulus indicates the inhomogeneous mechanical properties on the entire length of constructs and the over-crosslinking of the bottom layers. In contrast, the constructs fabricated via continuous FLight process show fewer differences from area to area. C. Tensile mechanical properties of FLight hydrogel constructs using different strategies. Constructs made by continuous FLight process feature better tensile mechanical properties (higher yield strain).

FIG. 26 A. Light dose test of photoresin mixed with varying concentrations of photoabsorber (Sunset yellow FCF) and the length of hydrogel constructs created with same light dose. The length of hydrogel constructs (i.e., crosslinking depth) is decreased by increasing the photoabsorber concentration. B. Fluorescent images of microstructures (i.e., microfilaments & microchannels) in FLight hydrogel constructs with different concentrations of the photoabsorber (Sunset Yellow FCF). No difference in refractive index is found from different photoresin. No differences in microfilament diameter, microchannel diameter and microchannel fraction in the 3D volume are observed in 3D constructs created by different photoresin. The above results demonstrate the addition of photoabsorber will not change the microstructures in FLight hydrogel constructs.

FIG. 27 Photoabsorber is needed to control the light penetration depth into the photoresin, which prevents overcrosslinking of the previously crosslinked resin. This allows a larger working window w.r.t. the flow rate with the continuous resin feeding to achieve a shape index close to 1 (which implies high print fidelity). Scale bar in both figures is 5 mm.

FIG. 28 Adding photo absorptive dye and applying dynamic laser intensity improves the printing quality. The shape index of hydrogel constructs fabricated by different intensity setups and photoresin formulas. For example, Intensity 50-75 indicates the light intensity curve starting at 50% of full intensity and finishing at 75% during printing.

FIG. 29 Filamented light path in the Multi-FLight system. A. Light path as seen from the top. Light from a spatially coherent light source is collimated and guided to Digital Micromirror Device (DMD), when then projects a custom cross-section into the resin-filled cuvette. B. The light is either projected from the top or from the side by rotating the mirror R1. Further, the orientation of the microfilaments can be changed by changing the orientation of mirror R1 (for side projections), or R2 (for top-down projections).

FIG. 30 Procedure for altering microfilament orientation, and example constructs for demonstrating the versatility of the MultiFlight system. A. FLight biofabrication of complex hydrogel constructs using different (bio)photoresin. Up to 4 types of photoresin can be loaded into the vial during the FLight process. FLight hydrogel constructs containing physical/chemical/biological gradients can then be created by adjusting the filling of (bio)photoresin. B. Microfilament orientation within the constructs can be changed by rotating mirror R2 and synchronizing the translation and rotation of the cuvette.

FIG. 31 Changing the distance between the lenses can change the projection image size (in this case, it is 1× or 4× the original size of the projected image (1×1 cm²), which in-turn can affect the microfilament diameter. Higher the magnification of the image, higher is the diameter of the microfilaments and the microchannels (in-between the microfilaments).

FIG. 32 Image pixelation can introduce unique microarchitectures within the constructs. A. Projected pixelated images (1×1 denotes that black and white pixels alternate, 2×2 denote that black and white pixels alternate in sets of 2 pixels) can generate un-predicted patterns due to interference effects. Herein, the 2×2 pixel arrangement results in an inclined arrangement of the crosslinked resin instead of a checker arrangement. B. Cells seeded on top of the hydrogels featuring a graded microstructural framework can align along the microstructures imparted using the pixelization strategy.

FIG. 33 Inducing the microarchitectures in FLight hydrogel constructs using mesh as photomask. A. Metal mesh (grating) is added in front of cuvette in the FLight setup at different locations. B. The photograph of hydrogel constructs consisting of microarchitecture with a W265D50 mesh (the mesh size is 265 μm and wire diameter is 50 μm) and the fluorescent images of constructs after washing of uncrosslinked photoresin. 5% fluorescently labelled Gel-MA was used (0.05% LAP) in this demonstration, scale bar: 750 μm.

FIG. 34 Bright field (BF) images of the wire mesh used in FLight system as diffraction grating. Based on the placement of meshes/gratings along the light path, microarchitectures with varying dimensions can be created by a series of meshes (W63D36: the mesh size is 63 μm and wire diameter is 36 μm; W90D36: the mesh size is 90 μm and wire diameter is 36 μm)). The size and shape of microarchitectures are highly related to the design of meshes. Scale bars are 750 μm in the front view and 300 μm in the side view.

FIG. 35 Unique patterns of light transmitted through the grating system (such as Moire patterns) can create varying hydrogel constructs. For example, two wire meshes can be inserted into the optical setup of FLight device to create a moire pattern, thereby forming the hydrogel constructs with the same shape as the moire pattern input. In this system, 2 or more gratings can be rotated (i.e., angle shift between meshes), and/or the distance between gratings can be adjusted to induce varying patterns along the light path (results shown in FIG. 36).

FIG. 36 Based on the Moire pattern strategy explained in the previous figure, a variety of hydrogel constructs could be fabricated using different meshes/gratings placed along the light path. A. The light pattern generated from the FLight setup using single wire mesh as diffraction grating, and photography of the hydrogel construct. B. The unique light pattern was created by inducing the second diffraction grating in the FLight system with 0.4 π angle shift between 2 wire meshes in FLight system. The printed hydrogel construct was then fabricated according filamented light pattern transmitted through the grating. C. The light pattern was changed when the angle shift was decreased from 0.4 π to 0.2 π, and the photograph of hydrogel constructs shows the different hydrogel constructs were made. The BF image indicates the microfilaments in the FLight hydrogel constructs.

### EXAMPLES

### Example 1: Optical Modulation Instability and Self-Focusing Induce Beam-Like Microstructures in Photoresins

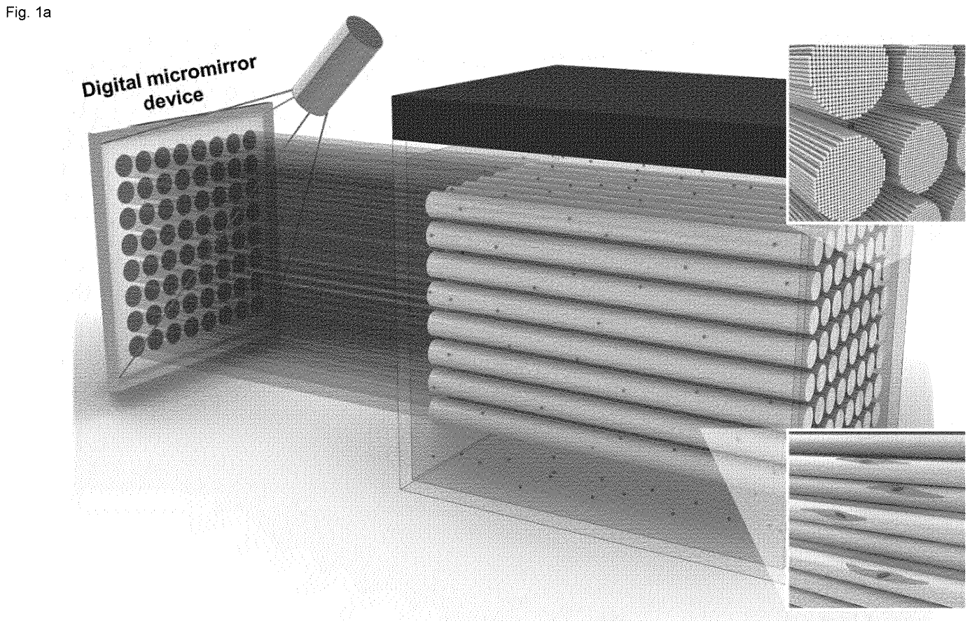

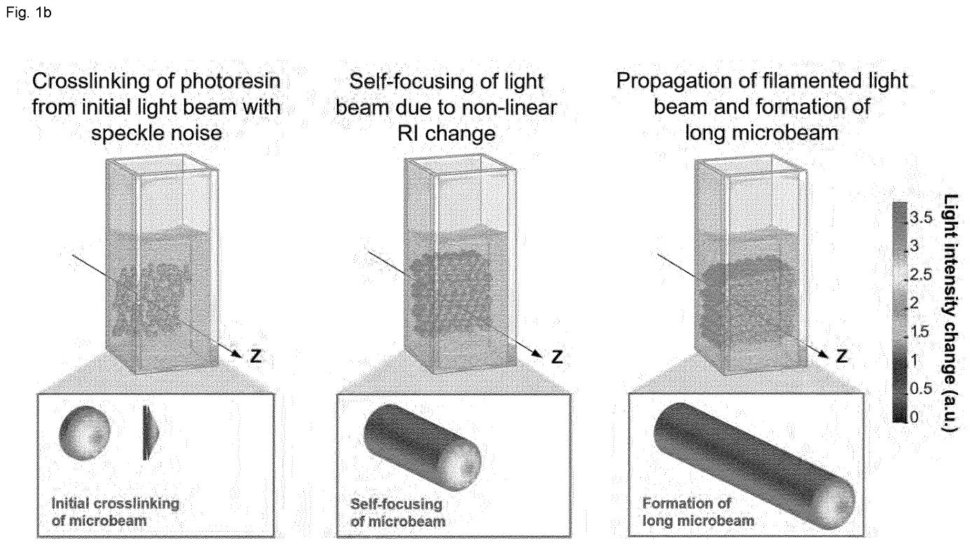

The physical phenomenon behind LBP's ability to form cell instructive microbeams from photosensitive resins is illustrated in FIG. 1a. The projected light beam features intensity noise patterns that translate into arrays of microbeams which make up the hydrogel structures. First, the inventors highlight the physics that gives rise to formation of these microbeams within the photoresins. The image projected onto the transparent photoresin-containing cuvette features a speckle-pattern intensity noise originating from the spatial coherence of the input light source. This small intensity noise is then non-linearly amplified by the crosslinking of photoresin, resulting in microscopic resin waveguides that propagate the filamented light beam front through the resin volume (i.e., cuvette inner thickness). More specifically, the initial local intensity noise maxima locally crosslink the photoresin from cuvette-photoresin interface, thus inducing a localized increase of RI in the photoresin. In turn, the photoresin RI changes result in a self-focusing effect at the interface between crosslinked and uncrosslinked photosensitive material in the z-direction. The formation of local RI maxima also creates local micro-waveguides which optically trap the incident light beam front and propagate into the photoresin over a long distance. In other words, the trapped and focused beams further crosslink the photoresin, thus continuously inducing the self-focusing effect and creating long microbeams within seconds. The cumulative effect of multiple local intensity maxima in proximity to each other produces in an array of aligned microbeams in the created hydrogel constructs. Interestingly, the formation of hydrogel microfilaments has also been observed in a previous volumetric 3D printing study. However, these microstructures were considered defects of volumetric 3D printing, and their potential values in the engineering of anisotropic tissue constructs has not yet been realized. Moreover, the un-crosslinked photoresin between the microbeams is present due to the local minima of intensity noise, leaving behind high-aspect-ratio microchannels between the microbeams after washing. The self-focusing and self-propagation of the microbeams within the photoresin is illustrated in FIG. 1b. It is important to note that spatial coherence in the incident light can be optically tuned, while generation and propagation of microbeams are dependent upon optical non-linearity properties of the resin (i.e., RI changes upon photo-crosslinking), thus altering characteristics of the microbeams. In the next section, the inventors discuss how the dimensions of the microbeams from different photoresins can be tuned.

### Example 2: Microbeams for Cell Guidance are Highly Aligned and Tunable in Dimension

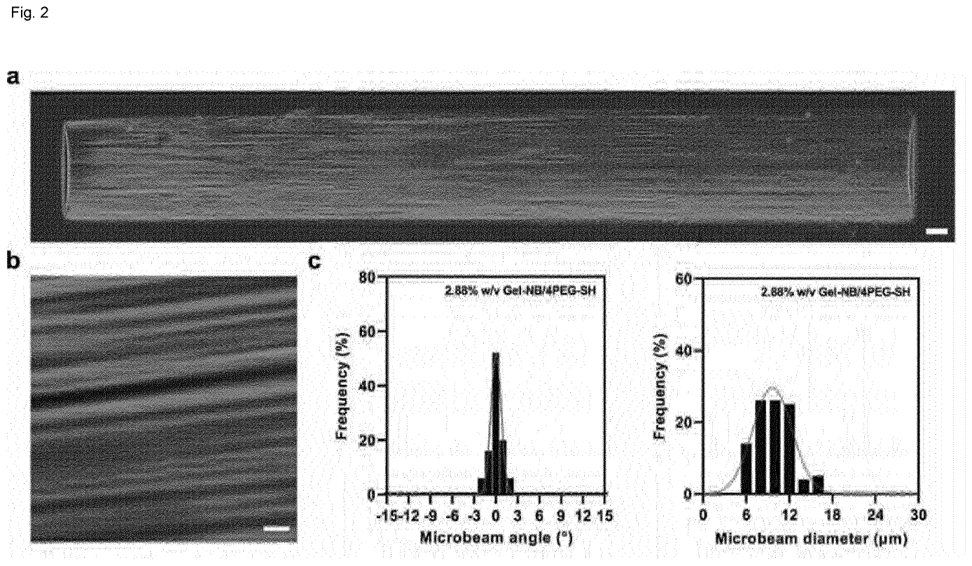

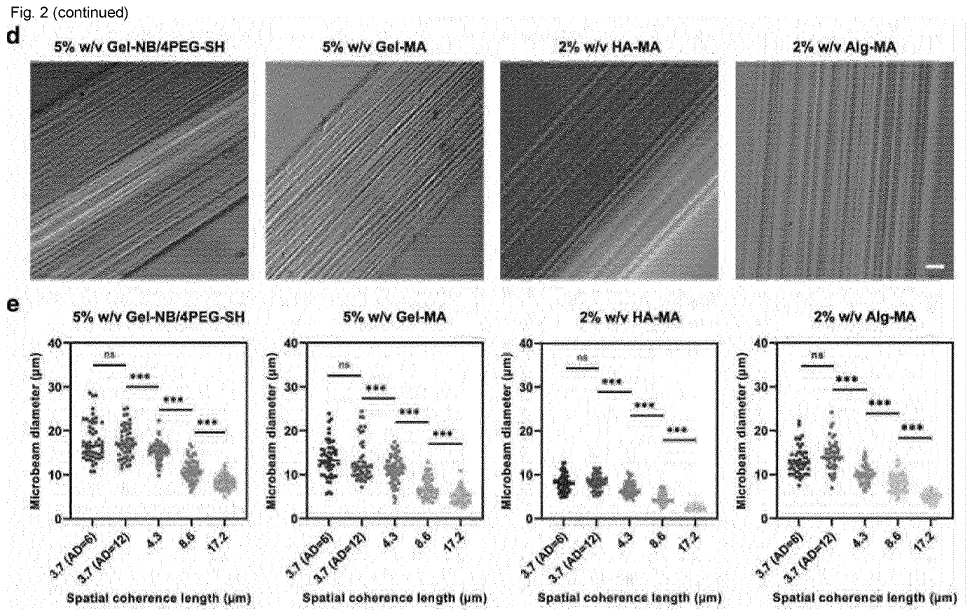

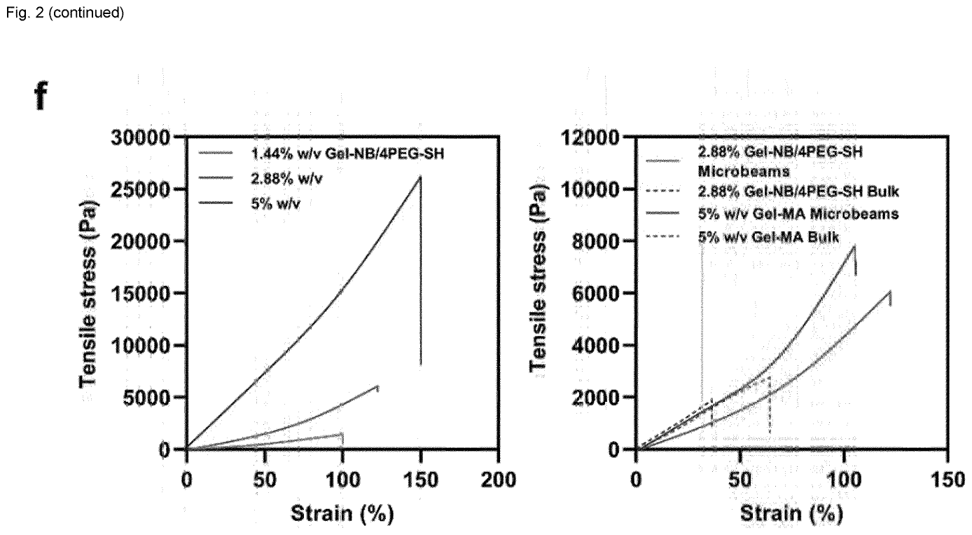

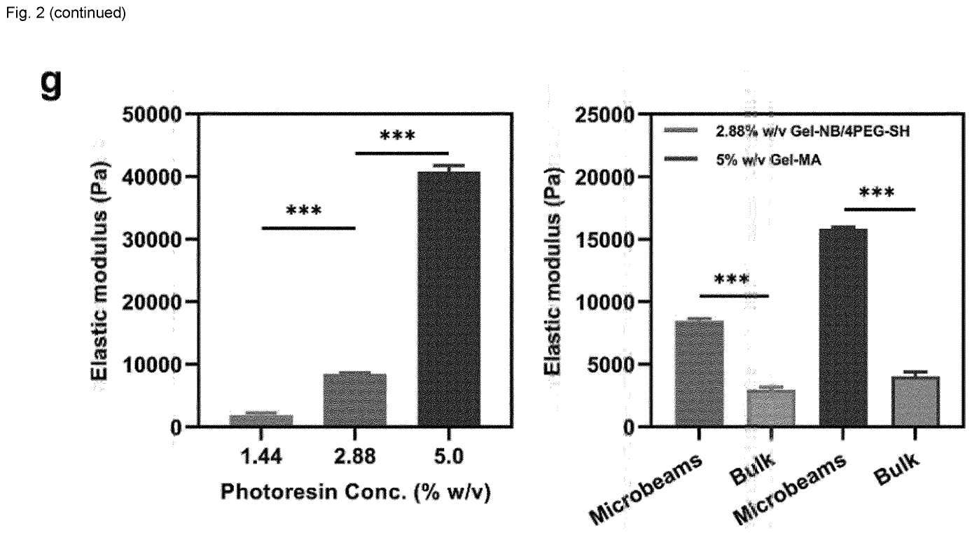

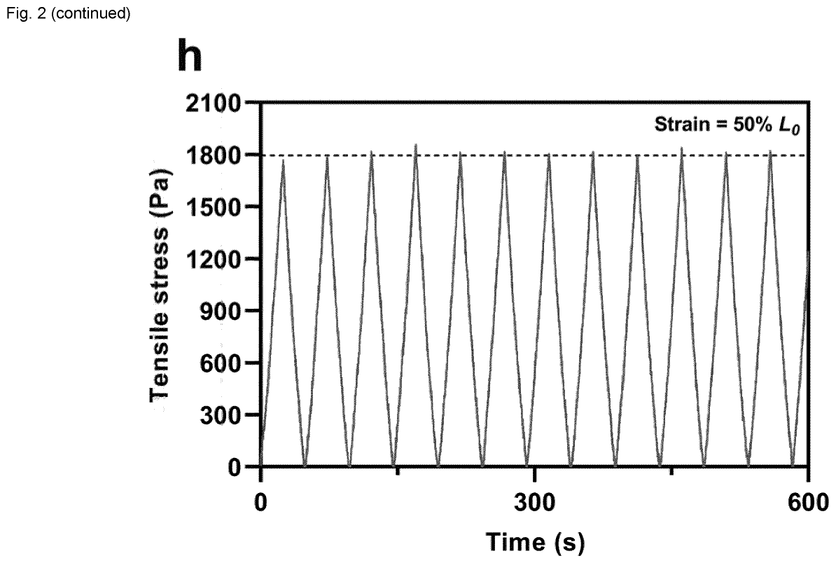

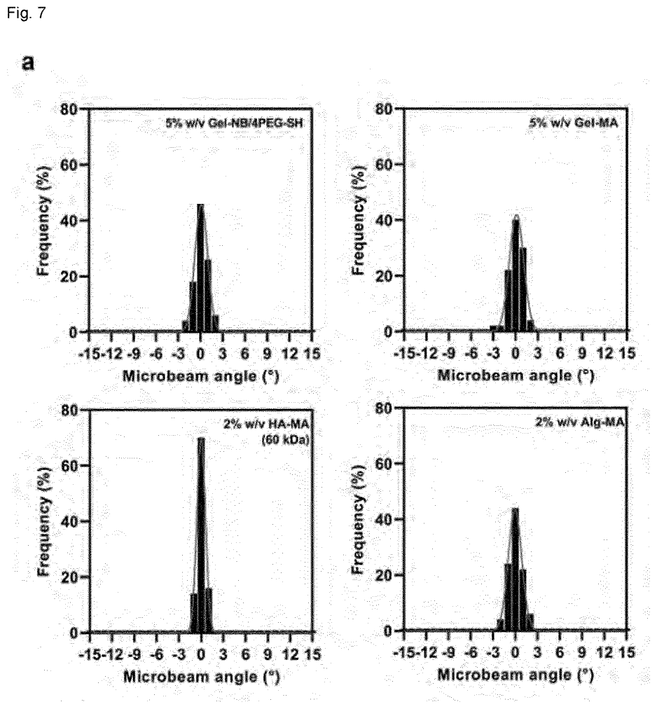

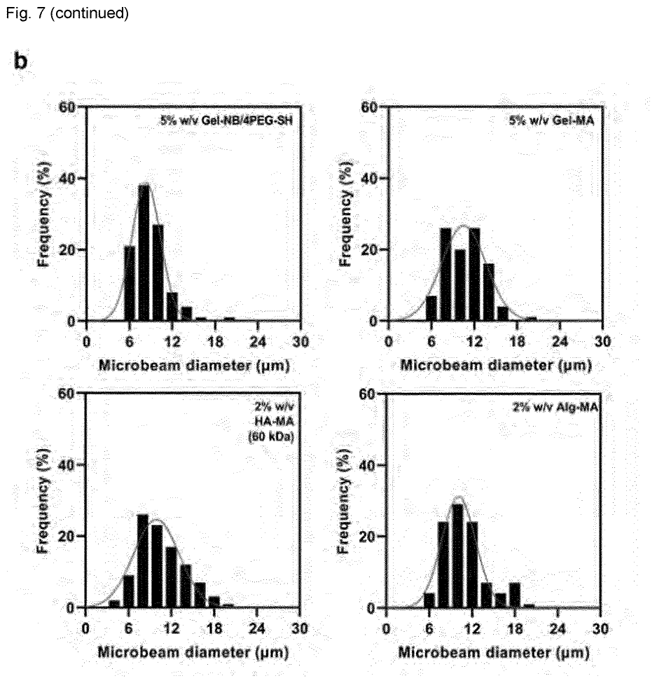

LBP was demonstrated for a several photosensitive resins. Due to the excellent kinetics of the norbornene-thiol reaction, microbeams were able to be formed within seconds, thus minimizing potential cell damage from photocrosslinking and allowing for the use of the low polymer concentrations. The inventors first produced hydrogel samples using Gel-NB/4PEG-SH photoresin. The critical light dose for projection was determined by performing a light dose test based on previous work. For the 2.88% w/v Gel-NB/4PEG-SH photoresin, a light dose of 195 mJ/cm² was used to create an LBP hydrogel structure with high fidelity to the designed dimensions (FIG. 7, Table 1). A 1 mm-diameter hydrogel cylinder was projected (FIG. 2a) and the formation of microbeams (diameter of 9.8±2.5 μm) within the cylinder was confirmed (FIG. 2b). ~88% of the microbeams were parallel (−1° to 1°) to the projection direction (FIG. 2c).

TABLE 1
Characterization of varying photoresin and microbeams structures as depicted in FIG. 2e.

| | | | | | | Aligned |
|---|---|---|---|---|---|---|
| | Polymer | Degree of | Microbeams | Refractive index | Critical | microbeams in |
| | Concentration | Substitution | diameter | (before | projection dose | target |
| Resin | (%) | (%) | (μm) | crosslinking) | (mJ/cm²) | degree, −1-1° (%) |
| Gel-NB/ | 2.88 | 47 | 9.8 ± 2.5 | 1.3395 | 195 | 88 |
| 4armPEG-SH | | | | | | |
| Gel-NB/ | 5 | 47 | 8.8 ± 2.4 | 1.3470 | 180 | 90 |
| 4armPEG-SH | | | | | | |
| Gel-MA | 5 | 45 | 10.7 ± 2.7 | 1.3435 | 675 | 92 |
| HA-MA | 2 | 20-50 | 8.1 ± 1.4 | 1.3375 | 5152 | 99 |
| 60 kDa | | | | | | |
| Alg-MA | 2 | 51 | 11.1 ± 3.1 | 1.3375 | 4758 | 90 |

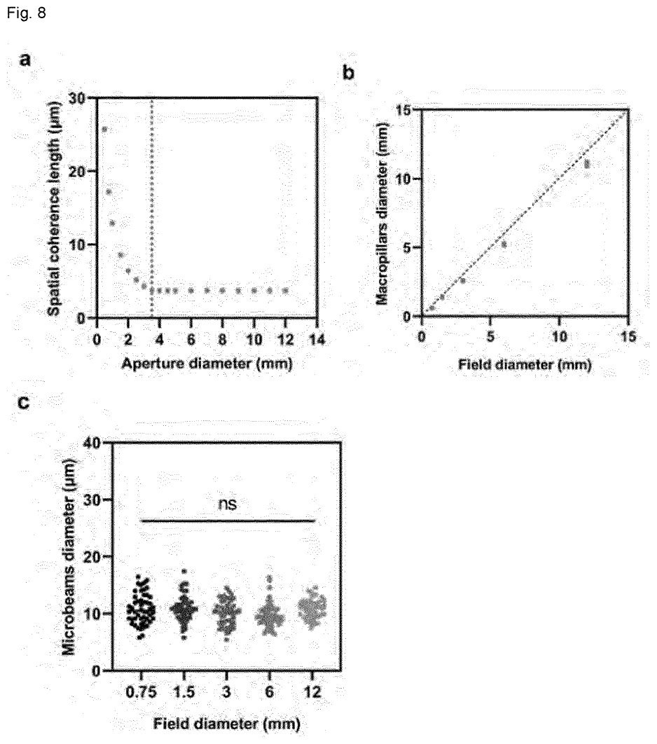

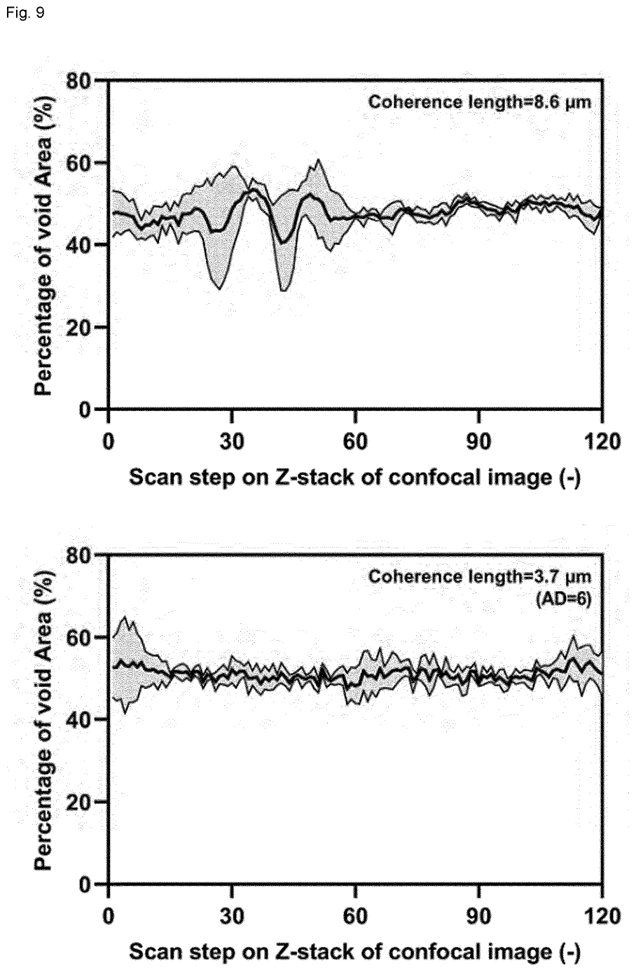

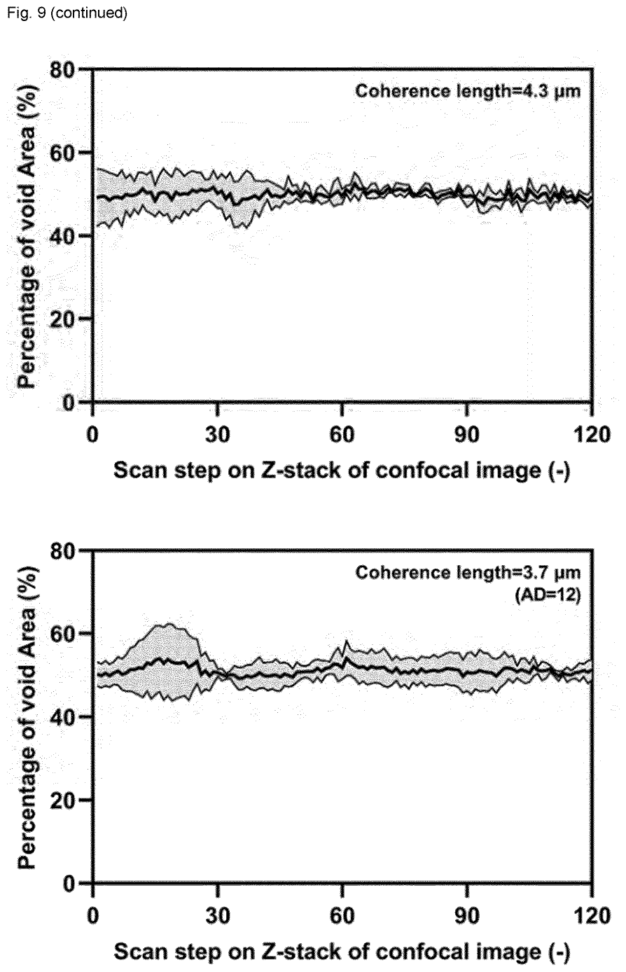

Since the generation of microbeams is dependent on spatial coherence of the light beams and nonlinear media, the inventors tested LBP using a higher concentration of Gel-NB/4PEG-SH (5% w/v), as well as other common photosensitive biomaterials (gelatin methacryloyl (Gel-MA), hyaluronic acid methacrylate (HA-MA), and alginate methacrylate (Alg-MA)) (FIG. 2d). Microbeams structures were formed using all photoresins, with highly aligned microbeams in all cases (>86%, −1 to 1°). The projection dose varied between 180~5650 mJ/cm² depending on the resin, as did the diameter of the microbeams (8.1±1.4~11.1±3.1 μm) (FIG. 8-9, Table 1).

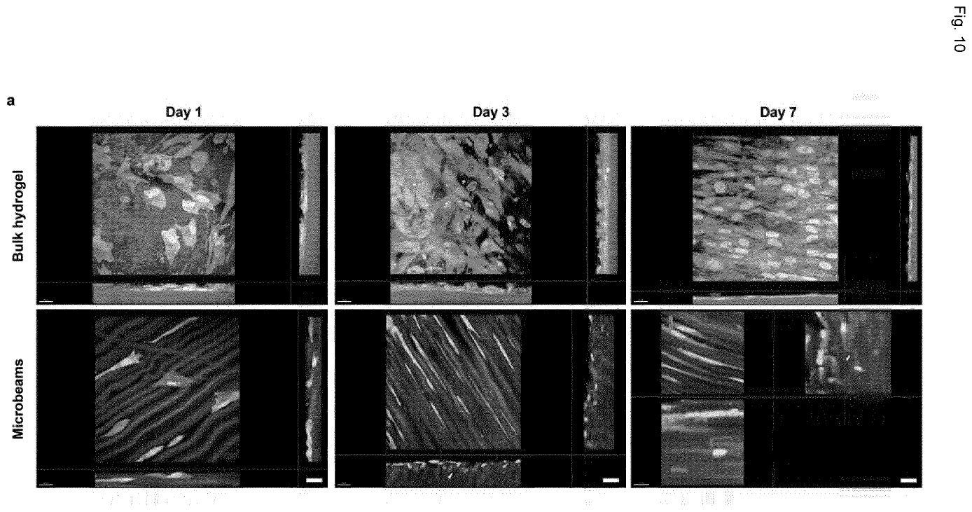

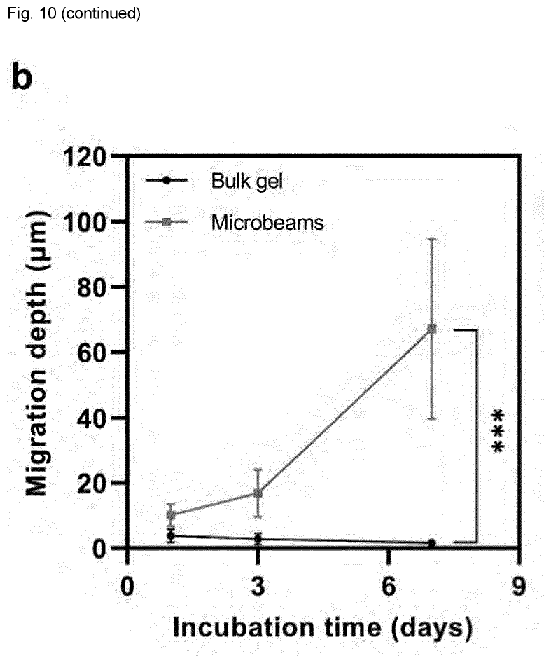

High-aspect-ratio structures can offer cell guidance, and the ability to tune their dimensions would make LBP applicable to a broad range of aligned tissue having ECM fibers ranging from 1 to ~100 μm. Here, microbeams with varying diameter ranges were fabricated by adjusting the spatial coherence length using a standard Köhler's illumination system consisting of a 405 nm LED light source. Specifically, this light-induced self-organization depends on spatial coherence of the light source and strength of the photoreactive material's RI nonlinearity. The more spatially coherent the light beam—in other words, the longer the spatial coherence length l_{c} of the light beam—the broader the diameter of the microbeams. In the inventors' Köhler illumination system, the spatial coherence length of the light beam could be spanned between the theoretical range of 3.7 to 25.8 μm (FIG. 10) by adjusting the aperture diameter (AD). This range of spatial coherence length allowed the inventors to control the diameters of microbeams between 8.3 and 17.8 μm using 5% w/v Gel-NB/4PEG-SH photoresin (FIG. 2e). The mean diameters of microbeams made from methacryloyl resins were 5.0 to 13.6 μm, 2.4 to 8.3 μm and 4.7 to 13.7 μm using 5% w/v Gel-MA, 2% w/v HA-MA, and 2% w/v Alg-MA photoresins, respectively. However, the mean diameter of microbeams was found to be independent of the field diameter of the inventors' Köhler system, which further demonstrates that the spatial coherence of the light beams induced by OMI controls the microbeam formation and that the microbeam diameter is tunable by adjusting the spatial coherence length of the light beam (FIG. 10).

Due to the reinforcing effect of the highly aligned fiber component, the hydrogel constructs composed of microbeams exhibit ideal mechanical properties for tissue engineering applications. Mechanical stimulation can be applied to such hydrogel constructs, which further induces cell alignment and boosts tissue maturation. Bulk hydrogel and microbeam-containing hydrogel samples were fabricated using Gel-NB/4PEG-SH or Gel-MA photoresin for tensile and compressive tests (FIG. 2f, g). The yield stress of hydrogel samples prepared using 2.88% w/v Gel-NB/4PEG-SH photoresin increased from 2.7 kPa (bulk hydrogel) to 6.0 kPa (microbeam-contained hydrogel), with yield strains of 64% and 122%. Similar results were found in the hydrogel samples produced using 5% w/v Gel-MA photoresin: the yield stress and yield strain were found to be 1.8 kPa and 36% in the bulk hydrogel sample. However, the yield stress and yield strain were increased to 7.8 kPa and 105% due to the presence of microbeams. The compressive elastic modulus of cylindrical LBP hydrogel constructs (a diameter of 5 mm and a height of 4 mm) increased from 1.8 kPa to 40.7 kPa, when increasing Gel-NB/4PEG-SH photoresin concentration from 1.44% to 5%. Likewise, the compression of LBP hydrogel samples revealed a higher elastic modulus compared to bulk hydrogel samples when tests were performed along microbeam direction; 8.4 vs 3.0 kPa for 2.88% w/v Gel-NB/4PEG-SH and 15.8 vs 4.0 kPa for 5% w/v Gel-MA photoresins, respectively. These differences in compressive mechanical properties between the bulk hydrogel and microbeam-reinforced hydrogel again indicated the reinforcing property of the microbeams in hydrogel constructs. Moreover, the deformation recoverability of the hydrogel sample was investigated by cyclic loading-unloading tests as shown in FIG. 2h. No residual strain was generated after loading-unloading during 10 mins of stretching at a tensile strain of 50% in the air condition, which suggests that hydrogels containing microbeams did not undergo plastic deformation upon tensile loading.

### Example 3: Channel-Like Voids Guide Anisotropic Cell Migration

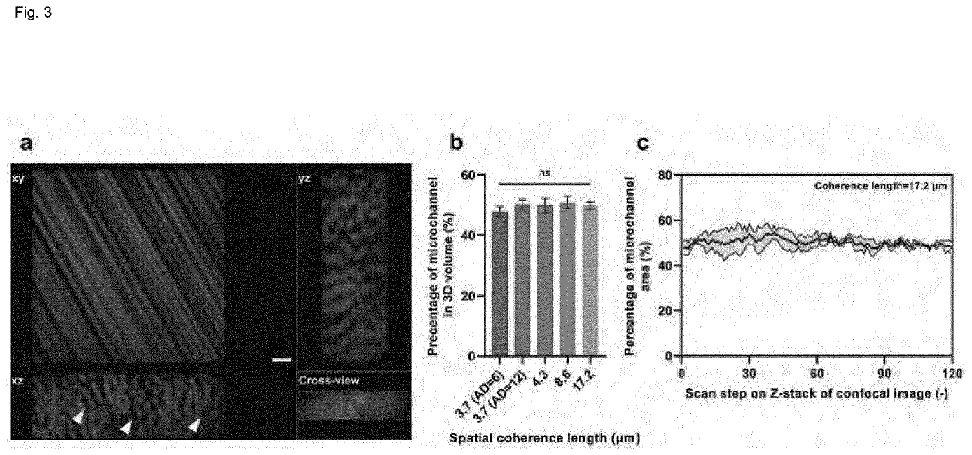

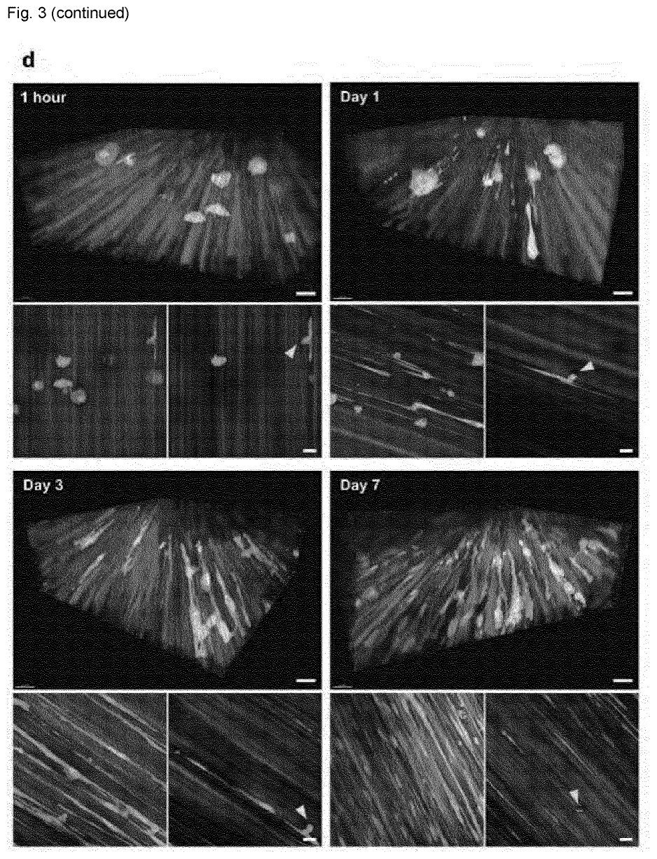

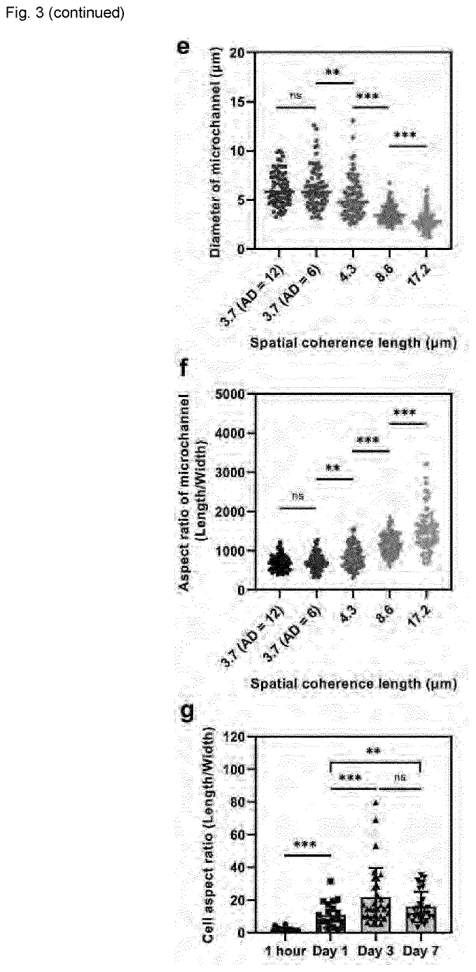

The nano-scale pore size of conventional hydrogel networks hinders the diffusion of oxygen and nutrients and lacks cell guidance cues. Micron-scale interconnected void spaces should be present in an ideal tissue engineering matrix, to provide 3D spaces for cell migration, proliferation, vascularization, and ECM deposition. Interestingly, a large fraction of the LPB-fabricated 3D matrix is occupied by microchannels with ultra-high aspect ratio (>700:1). These microchannels offer a 3D space into which the cell can migrate, and which causes alignment of the cell and its nucleus. The fluorescent-labeled microbeams were fabricated using 5% w/v fluorescent-labeled Gel-MA photoresin with subsequent washing of un-crosslinked photoresin. A large number of microchannels were observed in-between the microbeams, highlighted as white arrows in FIG. 3a. The void fraction was found to be about 50% in the whole hydrogel volume and independent of the spatial coherence length of light beams (FIG. 3b). Microchannels were homogeneously distributed within the hydrogel volume by measuring the ratio of microbeams/microchannels for each layer in the 3D images (FIG. 3c, FIG. 11).

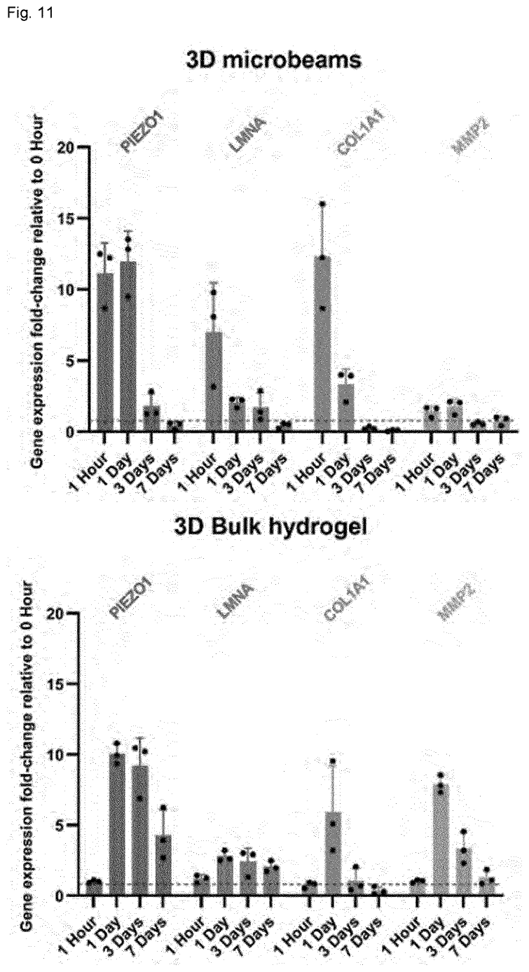

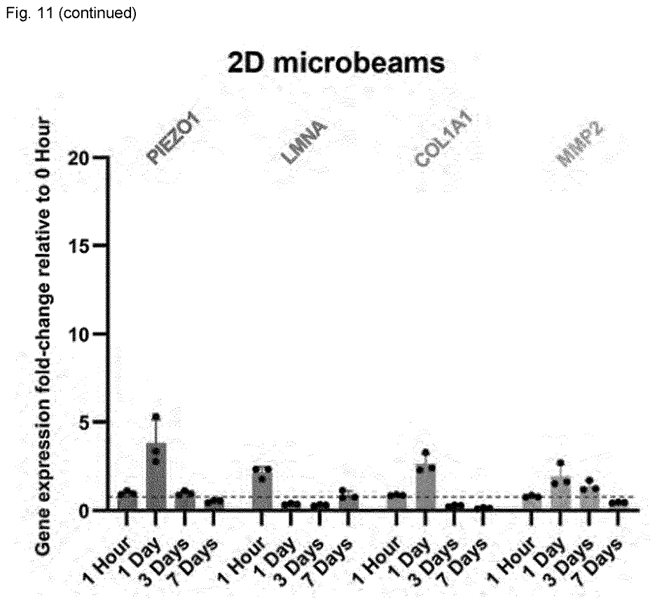

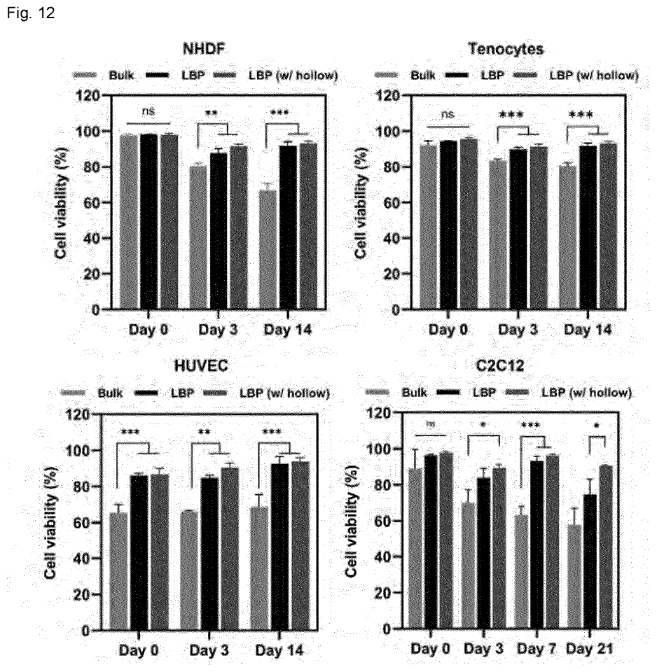

The physical limitation provided by pores smaller than 3 μm has been shown to significantly restrict cell migration in a 3D tissue matrix. This size is considered to be the threshold through which cells cannot pass, due to the limited deformability of the nucleus. Here, the diameter of microchannels was tuned by adjusting the spatial coherence length of light beam to ensure effective cell migration through the microchannels. The mean diameter of the microchannels was increased from 2.7 μm to 5.8 μm when decreasing the coherence length from 17.2 μm to 3.7 μm using 5% w/v fluorescent-labeled Gel-MA (FIG. 3e). Meanwhile, the mean aspect ratio of microchannel structures decreased from 1540:1 to 700:1 with increases in the spatial coherence length (FIG. 3f). To achieve cell migration and a higher aspect ratio for efficient cell guidance, Normal Human Dermal Fibroblasts (NHDFs) were encapsulated in LBP hydrogels containing microchannels with an average diameter of 3.5 μm and an aspect ratio of 1178:1. The NHDFs were stained by Calcein-AM to evaluate their migration after 1 hour, 1 day, 3 days, and 7 days of culture. Surprisingly, already after 1 hour the NHDFs were observed to migrate from the original encapsulation site into the microchannel with deformation of the nucleus (highlighted by yellow arrows in FIG. 3d). The mean aspect ratio of cells was increased from 1.24 to 15.8 after 7 days of culture (FIG. 3g), which demonstrates the elongation of cells during migration inside the microchannels. As evidence of interconnected microchannels in the hydrogel matrix (e.g., cells can migrate across microchannels), fibroblasts post-seeded on the surface of microbeams were observed to migrate into the hydrogel matrix from the surface after 1 day of incubation, but not from the surface of bulk hydrogels. The significant differences in cell migration depth show that these microchannels are interconnected to allow cell migration across the microbeams (FIG. 12). Overall, the above results demonstrate that these continuous, interconnected microchannels can support cell migration in the microchannel direction as well as across the microchannel direction. Such cell migration is essential for establishing cell-cell contact in a 3D matrix, such as the 3D network of tenocytes that requires gap junction-mediated intercellular communication. Moreover, microchannels can induce elongation of nuclei and support the nuclear migration, contributing a critical action in maintaining cell phenotypes and accelerating the fusion of myoblast for cellular reconstruction after muscle physiological damage.

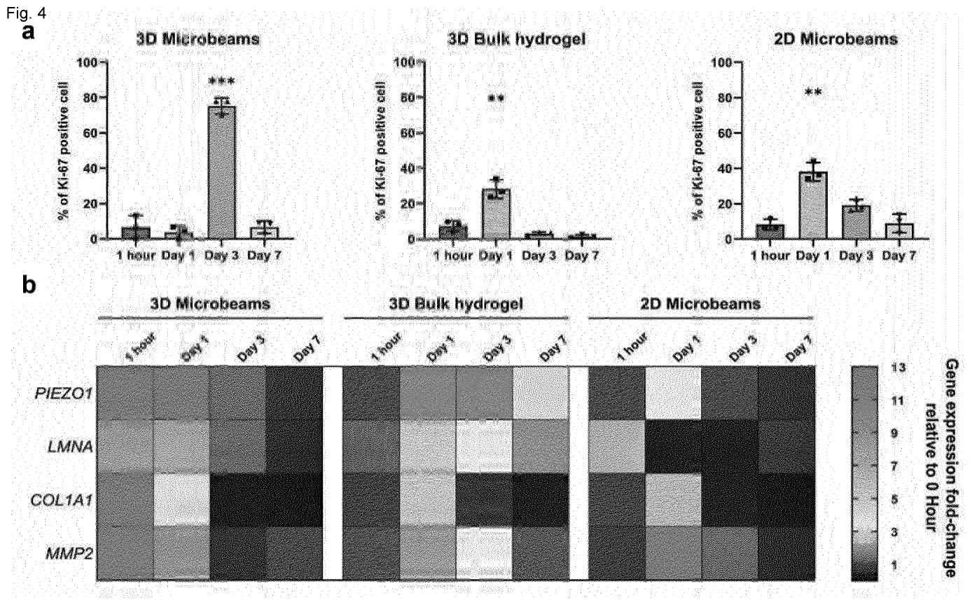

The regulation of gene expression resulting from reorganization of the nucleus upon mechanical confinement has shown interesting results in the tissue engineering field. For example, nuclear confinement can induce redifferentiation and reprogramming of cell fate by activating DNA repair pathways. At the tissue level, ECM-related gene expression is upregulated in cells with elongated nuclei under dynamic mechanical loading. In this study, the inventors demonstrated that ultra-high-aspect-ratio microchannels have unique advantages for creating highly aligned tissues by inducing mechanical confinement. Different tissue microenvironments were created using the same bioresin formulation (2.88% w/v Gel-NB/4PEG-SH photoresin with NHDFs), including the 3D microbeams, 3D bulk hydrogel, and 2D microbeams. The cells were encapsulated in the hydrogel construct containing 3D microbeams, in the bulk hydrogel, or post-seeded on the surface of microbeams (2D microbeams). The alignment of cells under different conditions was determined by phalloidin staining after 1 hour, 1 day, 3 days, and 7 days of culture (FIG. 4a). The cells started aligning along the microbeams after 1 day of incubation in both 3D and 2D conditions, but not in bulk hydrogel, which again demonstrated the cell guidance property of microbeams as physical cues. Compared to 2D microbeams, NHDFs encapsulated within the 3D microbeams matrix exhibited a lower proliferation level (ratio of ki67 positive cells) on the first day. However, approximately 76% of the NHDFs were found to be ki67-positive cells after 3 days of culture, while this ratio was about 19% on the surface of microbeams (FIG. 4b). Similar results have been reported on rejuvenating and proliferating states of fibroblasts after static compressive force and laterally restricted culture. The mechanical stimulation obtained from the hydrogel matrix could offer a new way to induce cell proliferation, especially for aged cells with proliferation deficits, such as tenocytes and tendon stem/progenitor cells.

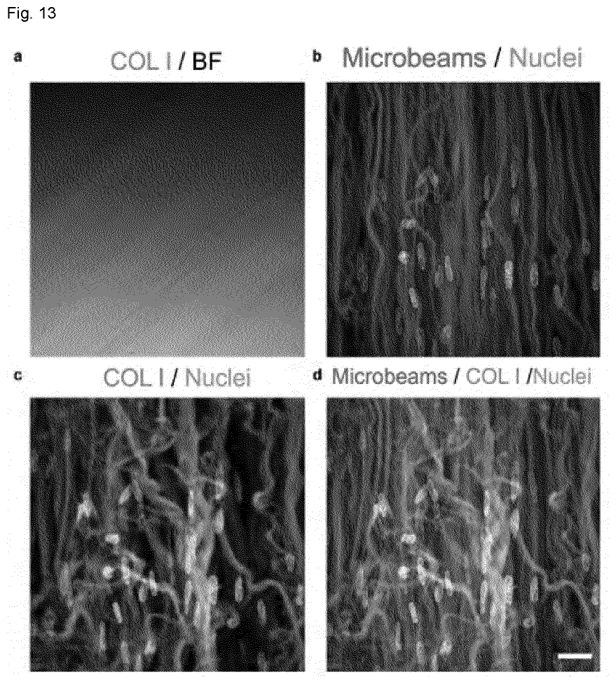

Next, the inventors demonstrated that ultra-high-aspect-ratio microchannels and the resulting cell confinement can regulate gene expression (FIG. 4c, FIG. 13). Upregulation of the mechanosensitive ion channel Piezo1 was found at the initial stage (1 hour and 1 day) in 3D microbeams conditions, compared to the 3D bulk hydrogel and 2D microbeams. Similarly, the inventors observed an upregulation of Lamin A/C proteins expression (encoded by LMNA gene) in 3D microbeams at 1 hour; this gene has integral roles in mechanical signals transmission from the cell membrane into the nucleus and in nuclear stiffness. Furthermore, COL1A1 expression was upregulated at the beginning of incubation, similar to PIEZO1 expression. Earlier studies have demonstrated the relevance of Piezo1 to ECM-related gene expression. For example, the lack of Piezo1 leads to a decrease in COL1A1 expression, and Piezo1 plays a critical role in ECM deposition during tissue maturation under mechanical stimulation. Also, the lack of these micron-sized voids in bulk hydrogel leads to higher MMP2 expression levels to aid cell migration by remodeling the hydrogel matrix. However, the decrease in PIEZO1 expression was found from day 3, which could be explained as a loss of mechanical stimulation. It may be caused by the degradation of microbeams and the deformation of microbeams by cell contracting. In the future, it is expected that the loss of mechanical stimulation will be able to be offset by applying additional static/dynamic mechanical loading, which is promising for the biofabrication of matured anisotropic tissues.

### Example 4: LBP for Biofabrication of Highly Aligned Tissue Constructs

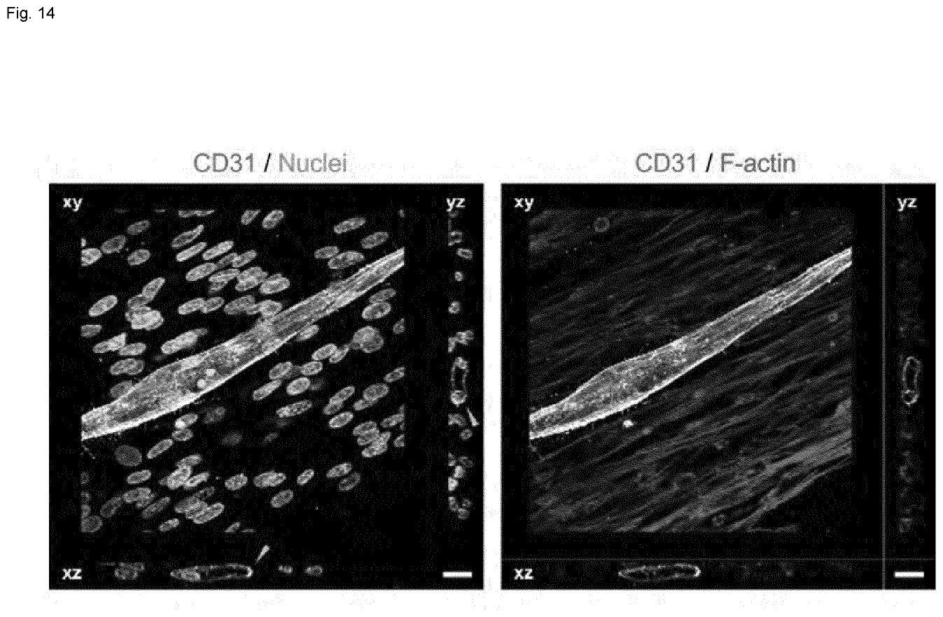

The inventors demonstrated ability of LBP to bio-fabricate highly aligned tissues using 2.88% w/v Gel-NB/4PEG-SH photoresin with four cell types: NHDFs, Human Tenocytes (HTs), Human Umbilical Vein Endothelial Cells (HUVEC) and Mouse Myoblasts (C2C12). Cell viabilities were found to be above 85% for all cell types 1 hour after LBP. By contrast, low cell viability of HUVEC (<70%) was observed in bulk hydrogels, which were prepared using the same light dose as LBP (FIG. 14). However, when the incubation was extended to 21 days, the viability of C2C12 was about 74% in the LBP hydrogel samples. To overcome this limitation, a series of hollow microstructures several hundred microns in diameter were created in hydrogel constructs by blocking light from the designed projection image. The encapsulated C2C12 cells showed higher cell viability (~89%) in hydrogel constructs containing multiple hollow channels after 3 weeks of culture. Therefore, multi-hollow microstructures (diameter of 216 μm) were employed in subsequent experiments to achieve higher bioactivity in cell-laden hydrogel constructs.

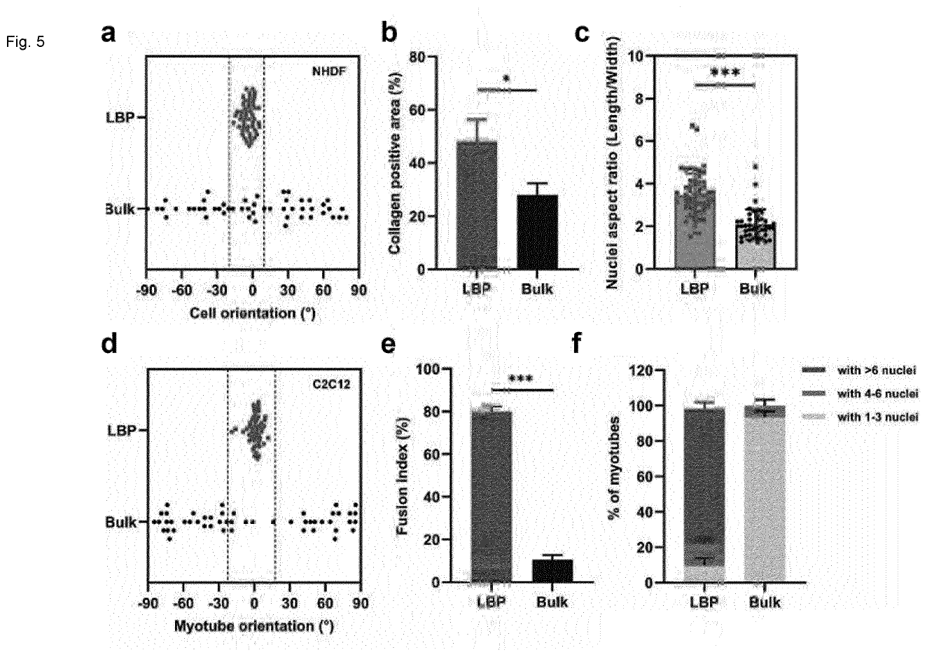

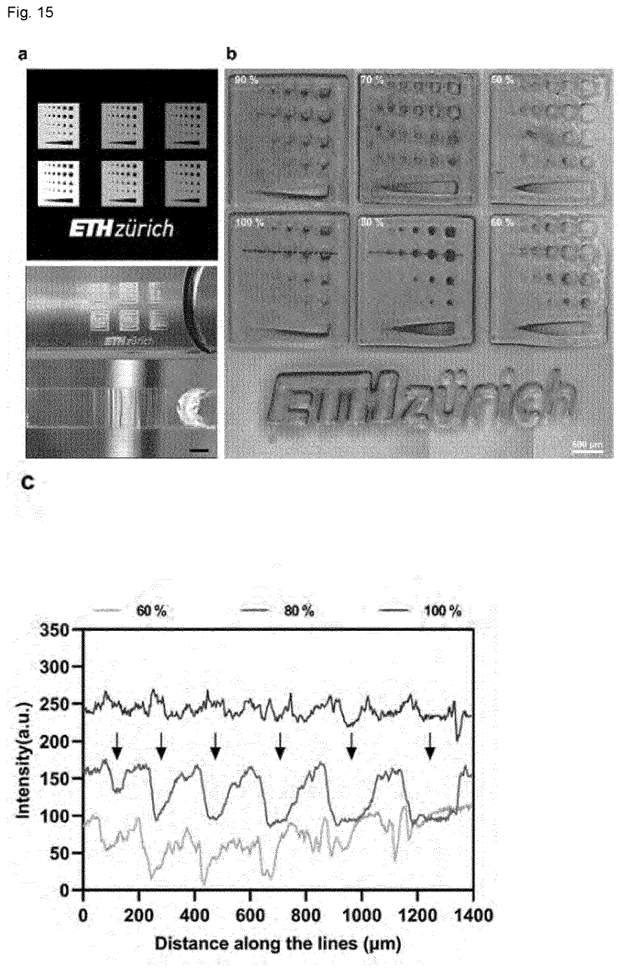

The potential of LBP in engineering anisotropic tissues is based on the microstructures' ability to guide cell alignment and on the alignment of ECM (i.e., self-aggregation into aligned collagen fibers). Using NHDFs, the cell-guiding property of the microbeams was tested in structured samples with a diameter of about 1 mm (light exposure 2.8 s, containing multi-hollow microstructures). Phalloidin staining was used to visualize the capability of microbeams to guide cell alignment (FIG. 5a). 99% of the NHDFs were elongated and aligned within +300 of the projection direction after 14 days of culture (FIG. 5e). The formation of aligned collagen type I was confirmed from the immunofluorescence images using an anti-collagen type I antibody (FIG. 15). On the other hand, no cell alignment and aligned collagen fibers were found in the bulk hydrogels due to a lack of cell-guiding cues (FIG. 16). Similarly, more collagen-positive areas (~48%) were found in hydrogel samples containing microbeams than in the bulk hydrogel (~28%), as shown in FIG. 5f. Interestingly, cell nuclei were also elongated and orientated in the microbeam-containing materials compared to nuclei of randomly orientated fibroblasts in the bulk hydrogel sample (FIG. 5g). Recent studies have shown that physical cues can regulate the shape of cells and nuclei, ultimately affecting the phenotype, cell behavior, and gene expression. For example, the presence of fiber components in tissue matrices can induce fibroblasts to switch their phenotype to a myofibroblastic state, characterized by elongated shape. Furthermore, laterally restricted cultures of fibroblasts induce de-differentiation and reprogramming, which leads to rejuvenation of aged fibroblasts.

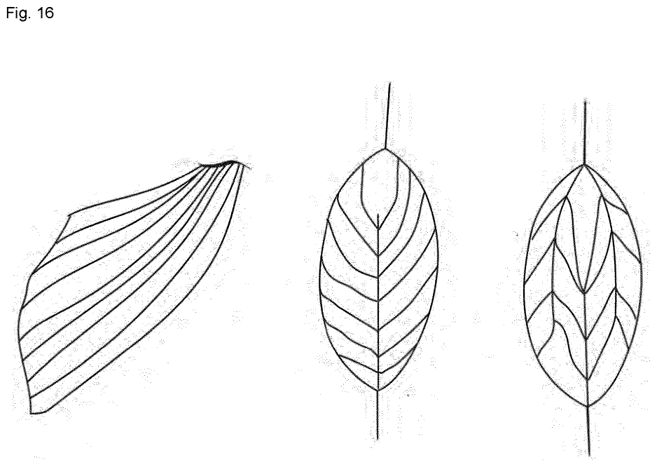

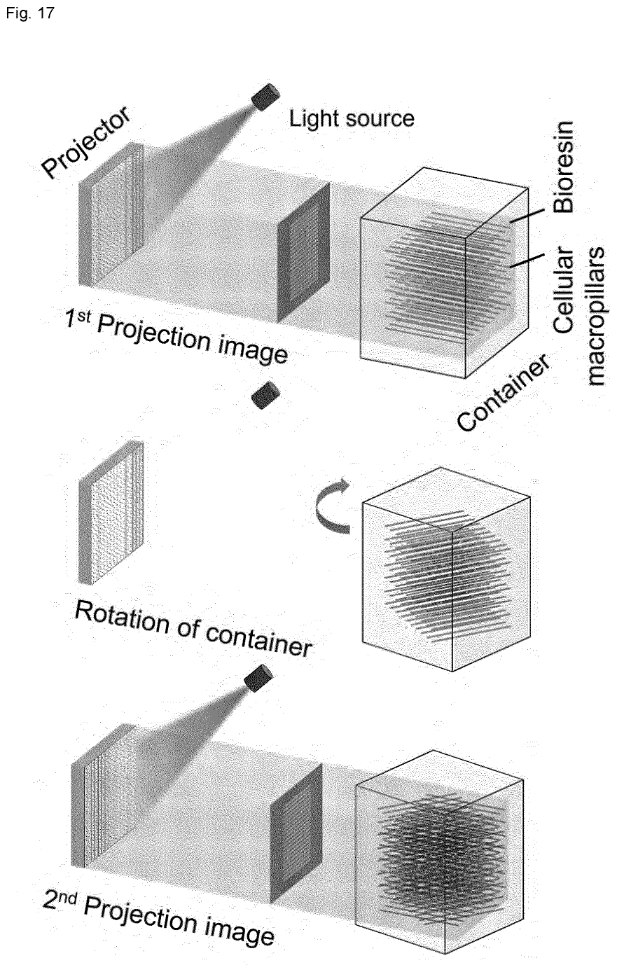

Next, the inventors studied the alignment of HTs in hydrogel matrix fabricated by LBP, which has important implications for the biofabrication of tendon tissue models. The highly aligned filamentous actins (F-actin) were found in HTs after 2 weeks of culture. Immunofluorescent detection of type I collagen fibers again demonstrated that the microbeams provide efficient guidance for cells and their deposited ECM (FIG. 5b). Additionally, by introducing a fibroblast-HUVEC co-culture system, the inventors showed that the formation of highly aligned blood capillaries could also be guided by LPB as detected by CD31 immunostaining in two-week cultures (FIG. 5c). Interestingly, the inventors found the continuous capillaries to be aligned in the projection direction, and some vasculatures were observed with a length >1.4 mm. Blood capillaries were confirmed to have lumen, as seen from a cross-sectional view of the 3D confocal image (FIG. 17).

Finally, C2C12 cells were encapsulated in 2.88% w/v Gel-NB/4PEG-SH photoresin and LBP was performed to further demonstrate its potential in the biofabrication of muscle tissues. Upon exposure to differentiation medium containing 2% v/v horse serum, the inventors observed the fusion of myoblasts and alignment of myotubes as seen with immunofluorescence staining using anti-mouse Myosin Heavy Chain (MyHC) antibody and phalloidin after 3 weeks of culture (FIG. 5d). 95% of myotubes were found to be aligned along the orientation of microbeams (−30 to 30°) within the LBP tissue samples (FIG. 5h). As expected, the myotubes showed a random orientation in the bulk hydrogel. Similarly, more highly aligned contractile myotubes with a higher fusion index were found in LBP samples compared to bulk hydrogels (FIG. 5i). It was interesting to observe that over 76% of the myotubes in LBP-fabricated hydrogel constructs had more than 6 nuclei per myotube (FIG. 5j), but not found in the bulk hydrogel samples.

Overall, the inventors have demonstrated that LBP is an effective strategy for the biofabrication of anisotropic tissue, with rapid manufacturing times and excellent cell biocompatibility. The cell-guiding physical cues, the microbeams, lead to efficient alignment of cells required to create anisotropic tissue. LBP offers a critical advantage over conventional strategies, which are often reliant on post-seeding of cells onto fiber-based scaffolds and substrates. That fact that these techniques merely provide a 2D microenvironment and inhomogeneous cell distribution further limits its applications. For extrusion-based bioprinting strategies, a high concentration of fibrillar components and smaller nozzles are often necessary to achieve effective cell alignment, which can generate significant shear stress on cells.

### Example 5: LBP for Biofabrication of Complex Hydrogel Constructs

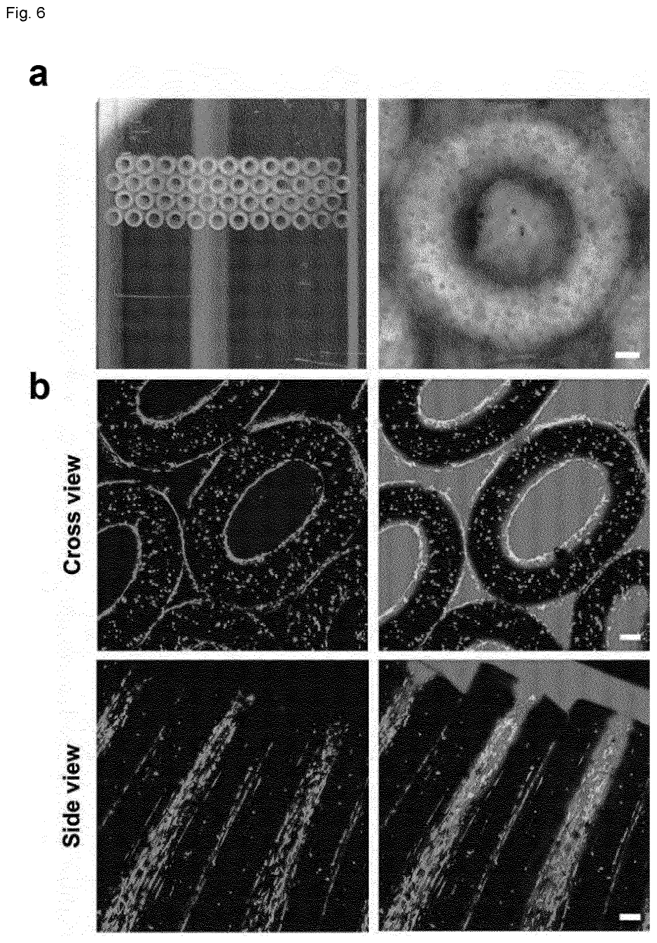

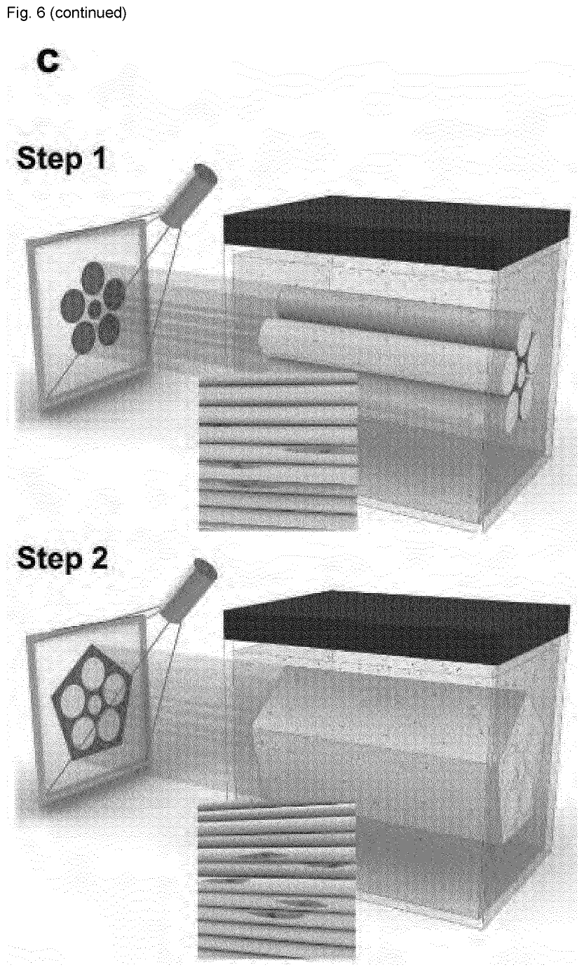

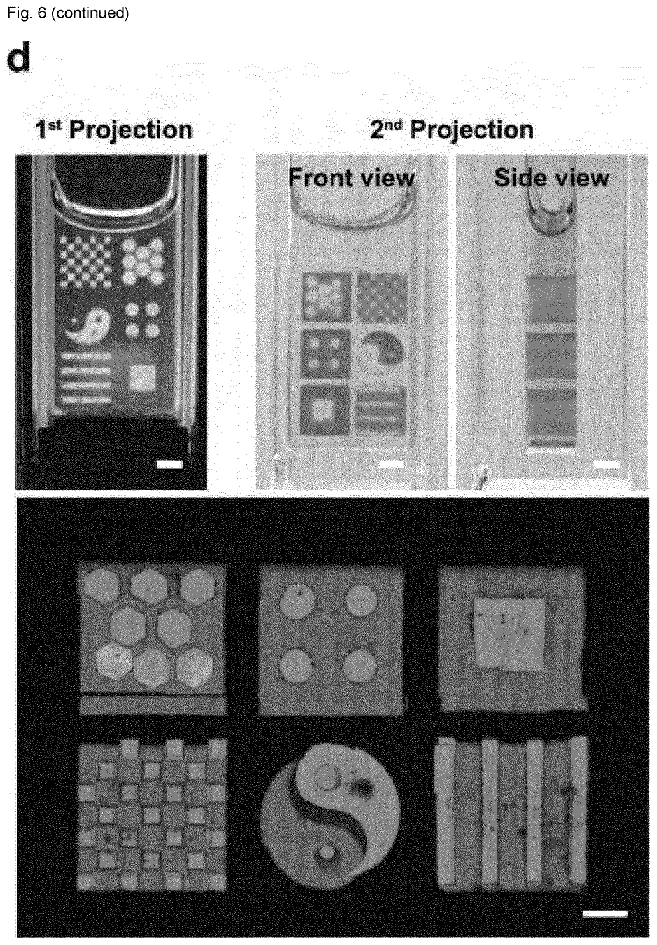

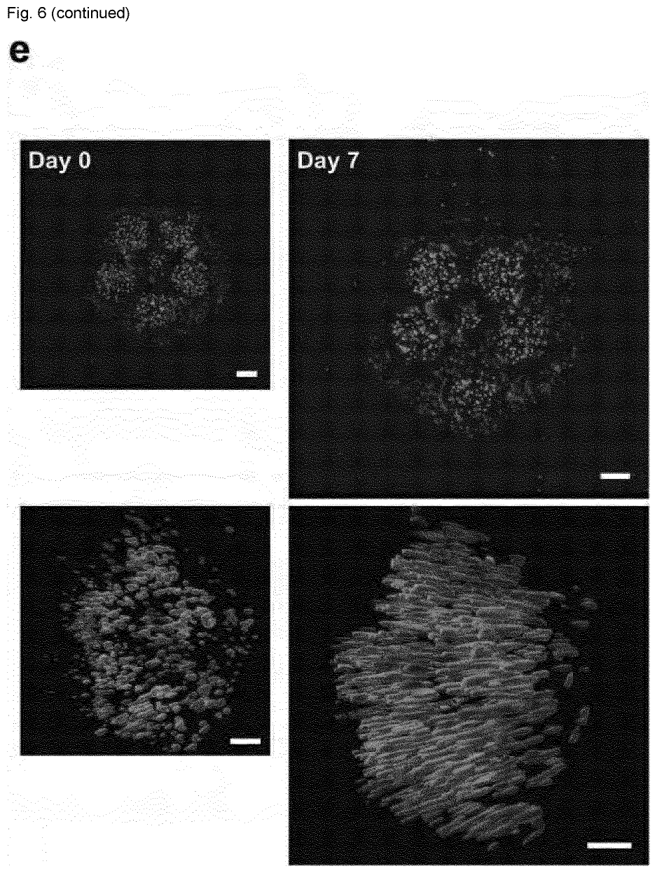

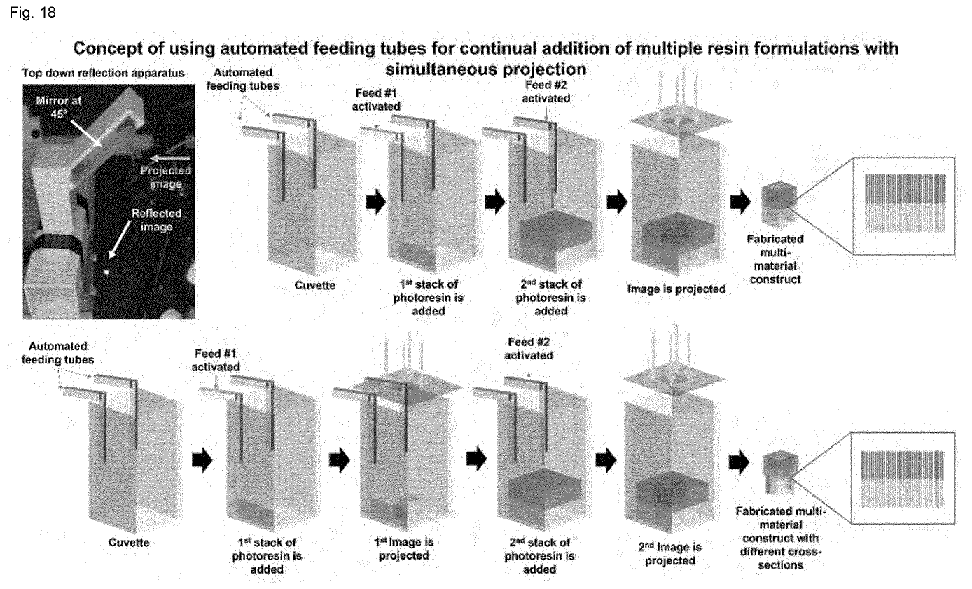

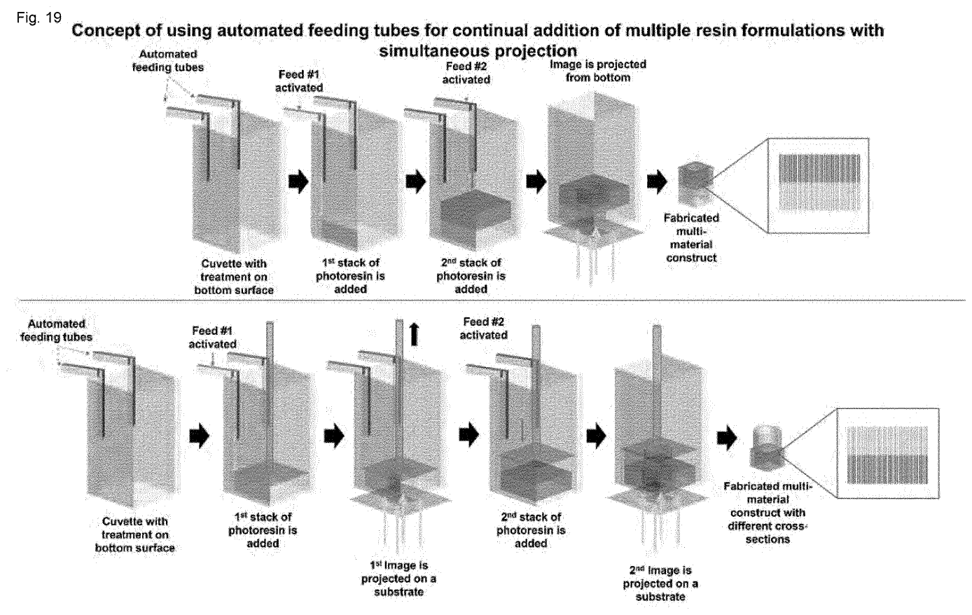

Lastly, the inventors explore the LBP's potential to create anisotropic tissues mimicking in vivo tissue structures. Varying hydrogel constructs with detailed features were produced using 2.88% w/v Gel-NB/4PEG-SH photoresin including hollow structures (FIG. 6a, FIG. 18) and a centimeter-scale hydrogel QR code with ~220 μm feature size. Also, cell-laden tubular hydrogel structures were created, and the encapsulated NHDFs were stained with Calcein-AM after 7 days of culture (FIG. 6b). Monolayers of NHDFs were observed on the inner and outer walls of the tubular structures. The subsequent filling of the channels with a fluorescent-label dye (red) indicated that these tubular structures were maintained after 7 days of culture. The above results demonstrated that LBP is a promising method to create anisotropic hydrogel constructs with free-designed cross-sectional structures for anisotropic tissue engineering.

A multi-cellular/multi-material biofabrication strategy was established by performing multiple sequential projections with an exchange of bioresin between projections. LBP allows for the creation of hierarchical organization of tissues, with control over the placement of cells and materials and material stiffness. As shown in FIG. 6c, multi-material structures were achieved with two complementary projection images. With the first image, the bioresin was spatially cured to create the first part of the hydrogel structure. After removing the un-crosslinked bioresin, the second bioresin was loaded into the vial and the second complementary image was projected. As demonstrated, acellular hydrogel structures were produced using two fluorescently-labeled photoresins (FIG. 6d). The cell tracker green-labeled NHDFs were mixed with 5% w/v Gel-NB/4PEG-SH photoresin and the central cylindrical hydrogel structures were projected. The peripheral tissue structures loaded with cell tracker red-labeled NHDFs were projected using 2.88% w/v Gel-NB/4PEG-SH photoresin. The successful encapsulation of two fluorescent-labeled cells into the designed hydrogel structure was confirmed (FIG. 6e). As expected, the alignment of cells in both projected hydrogel constructs was observed after 7 days of incubation, due to the presence of microbeam structuring in hydrogel constructs.

### Example 6: Multi-Direction Projection Strategy

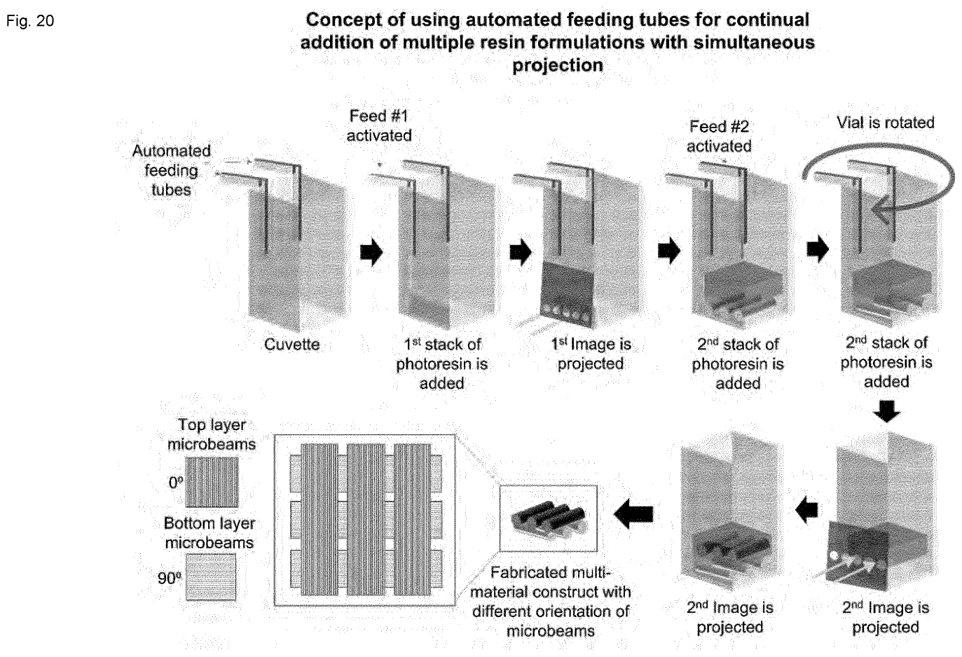

The inventors have developed projection strategy that allows the biofabrication of hydrogel construct containing microbeams in multiple directions. It is achieved by repeating the projection process from different directions. Briefly, the photoresin container is rotated by a specific angle for the second projection (e.g., 30 degrees, 45 degrees, 90 degrees) after first time projection (FIG. 20). This method will satisfy the biofabrication of anisotropic tissues that require specific topological guidance cues, such as myocardial muscle tissue.

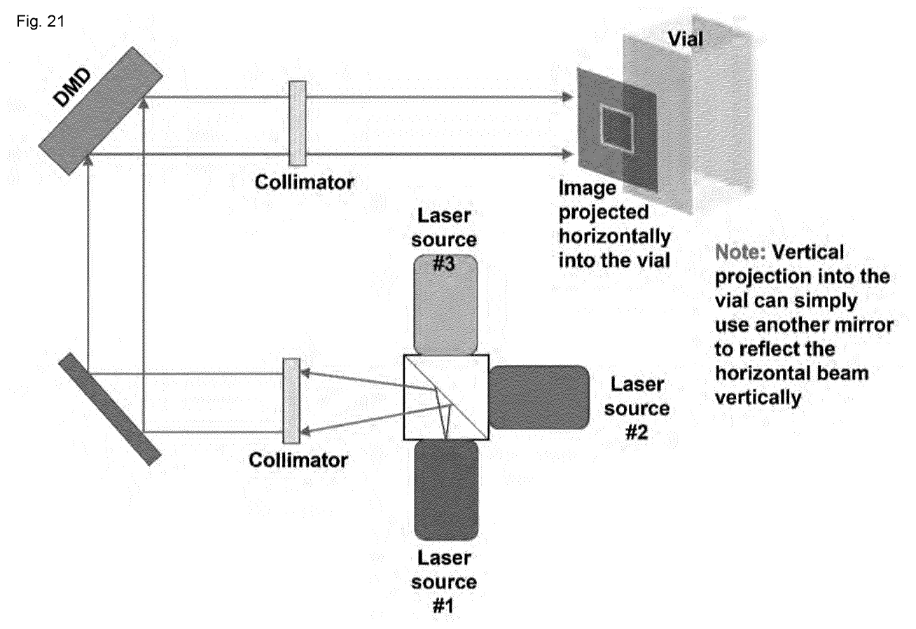

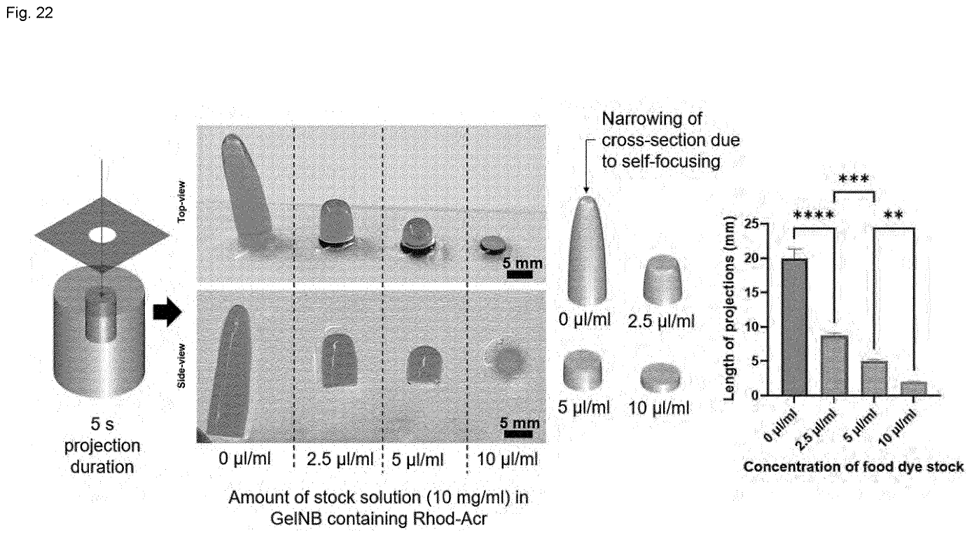

Next, a method for fabricating ultra-long hydrogel structures was demonstrated. This allows the fabrication of tissue structures with ~cm length and containing a continuous microwave beam. This enables the fabrication of hydrogel constructs of infinite length. The light beams come from the top of container (FIG. 21), or bottom of the container (FIG. 22). When the light beams come from bottom, the bottom surface of the container is treated to prevent the cured hydrogel structure from adhering. The light projected from below will crosslink the photoresin between the bottom surface of container and the substrate, thereby causing the hydrogel construct to adhere to the substrate. Then the substrate will move to the next position to allow new photoresin to be filled, with the followed projection to create long hydrogel constructs.

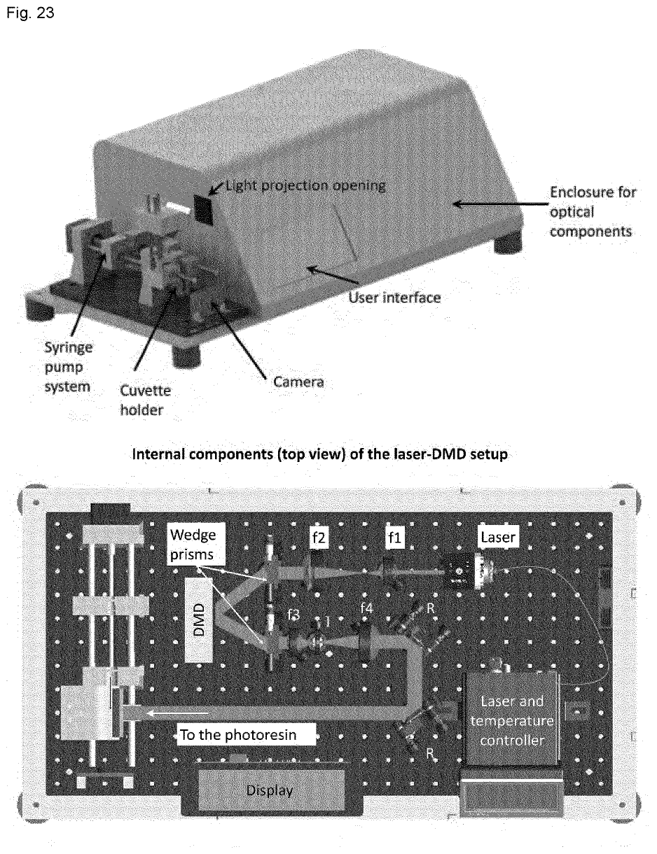

Finally, the inventor established hybrid projection method that combine the horizontal multi-direction projection strategy with the top projection approach (FIG. 23). This allows the produced hydrogel structures to contain, horizontally multi-direction aligned, vertically aligned microbeams.

### Example 7: Hybrid Biofabrication Strategy of Hydrogel Constructs (Multi-Materials/Multi-Light Sources)

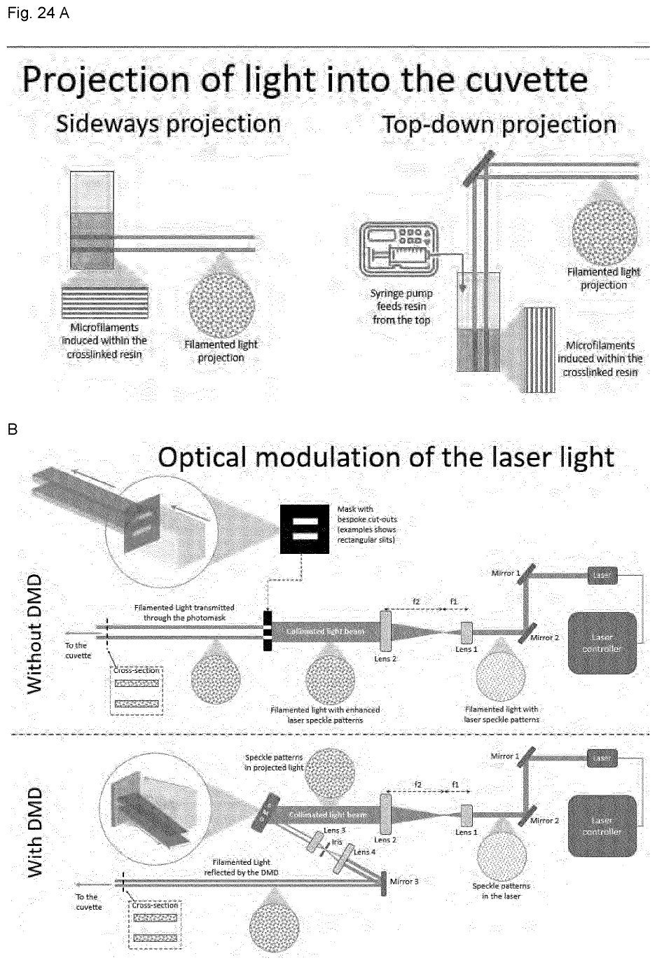

The inventors have demonstrated a new design for the light source (FIG. 24), and the optical setup, which allows our biofabrication strategy to be suitable for a wider range of photosensitive materials (e.g., photoreactive systems requiring different wavelengths to trigger the reaction). Varying optical setup further offers broader control on the dimensions of microbeams/microchannel, void fractions, etc. which will allow the fabricated hydrogel matrix to support as many or more complex tissue types. For example, a smaller microbeams drives better cell alignment, but larger (or more) voids effectively aid cell migration and establish cell-cell communication.

### Example 8: Long Microbeam-Laden Tissue Construct Fabrication at High Cellular Densities

The light scattering induced by high cell densities (>5 million cells/ml) during the projection often restrict the formation of microbeams to a few mm (typically 2 mm) along the length of the hydrogel construct. The inventors have demonstrated that using a refractive index matching agent (e.g., lodixanol at 30% w/v within the resin) to increase the refractive index of the resin (e.g., refractive index of 5% Gel-NB/Gel-SH photoresin increases from 1.345 to 1.46 after adding lodixanol to a final concentration of 30% w/v in Gel-NB/Gel-SH photoresin). This method can reduce light scattering and allow the formation of long and continuous microbeams in cell-laden hydrogel constructs with higher cell densities.

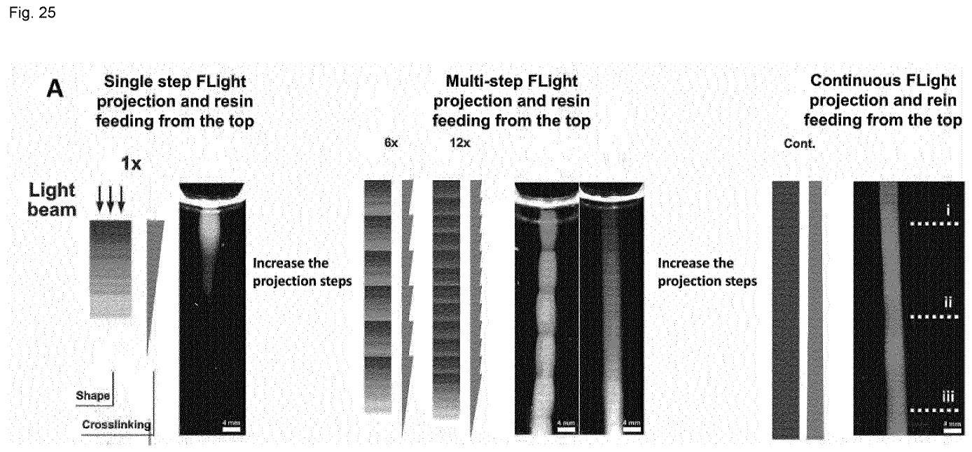

Further, for the fabrication of long hydrogel constructs, the inventors have used a bottom-up projection approach (as previously described in FIG. 21), wherein the first layer is added and crosslinked via the aforementioned projection approach, followed by the addition of subsequent photoresin and repeat the photo-crosslinking. This can be executed to virtually infinite number of steps to achieve a long (>20 cm in length) hydrogel construct. However, the inventors also observed that the projection of any layer would also expose the layer underneath to the receive additional light dose, thereby increasing the degree of crosslinking in the layer underneath. This will lead to inhomogeneous crosslinking across the entire length of the construct where the bottom layers typically feature high stiffness and merged microbeams, thereby demonstrating sub-optimal cell alignment and anisotropic matrix organization. The inventors have demonstrated that the photoresin could be supplemented with a photo absorptive dye (e.g., sunset yellow or FCF), which causes a finely-tuned penetration of the light into the photoresin (FIG. 25). For instance, the inventors calibrate the amount of the photoresin that is added for each layer and the associated light dose intensity and duration, such that the penetration depth of each projection matching the projection depth of the new layer. This causes and integrated construct to be formed without excessive photo-crosslinking in each layer, thereby resulting in cell alignment and anisotropic matrix organization throughout the length of the construct.

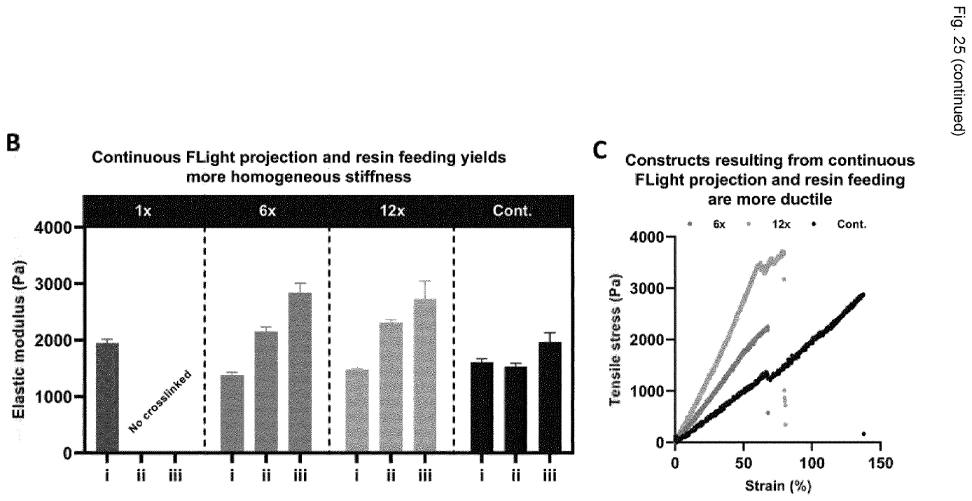

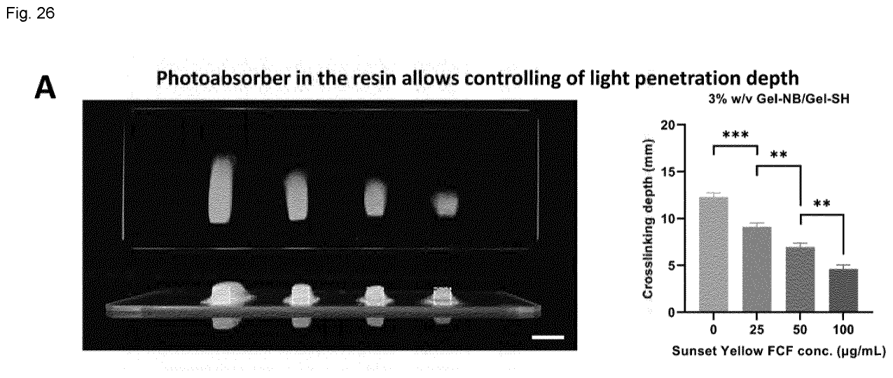

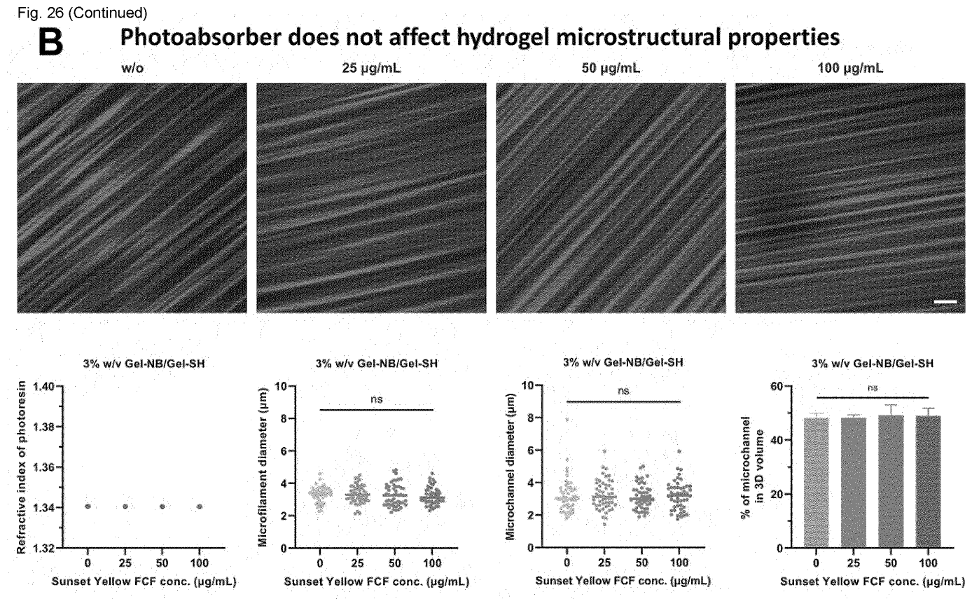

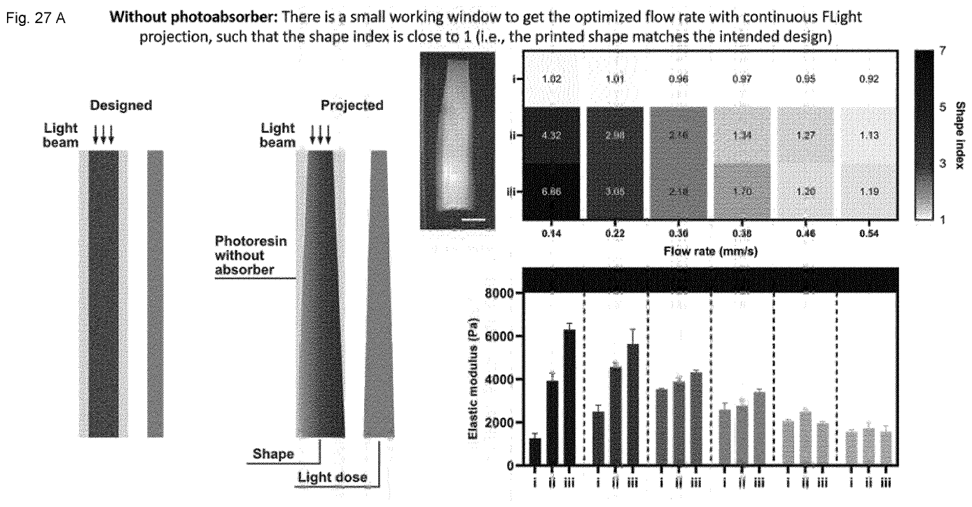

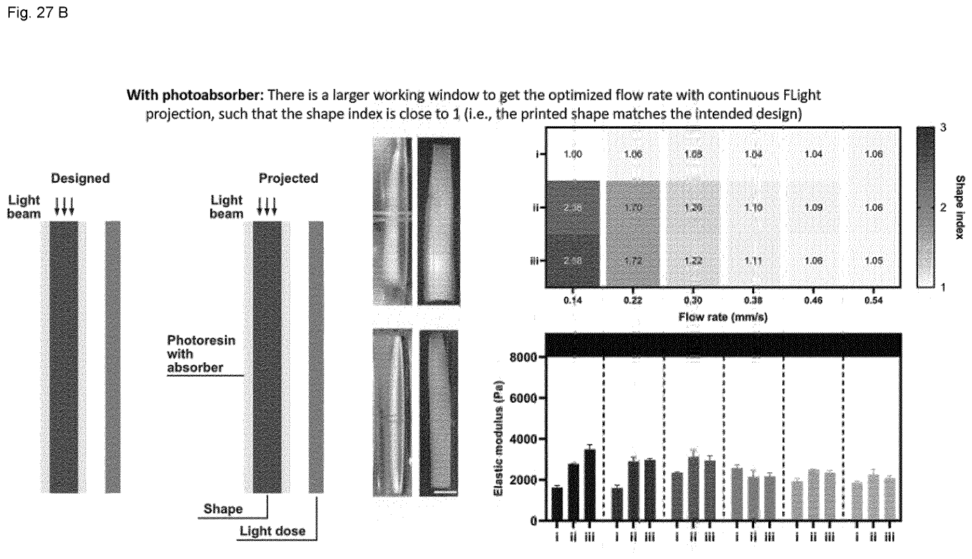

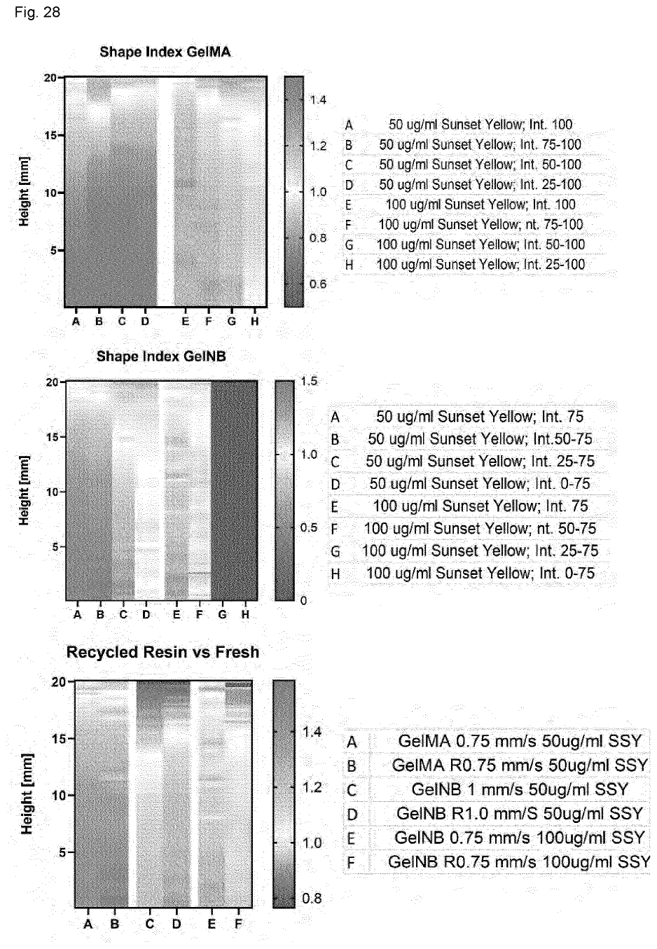

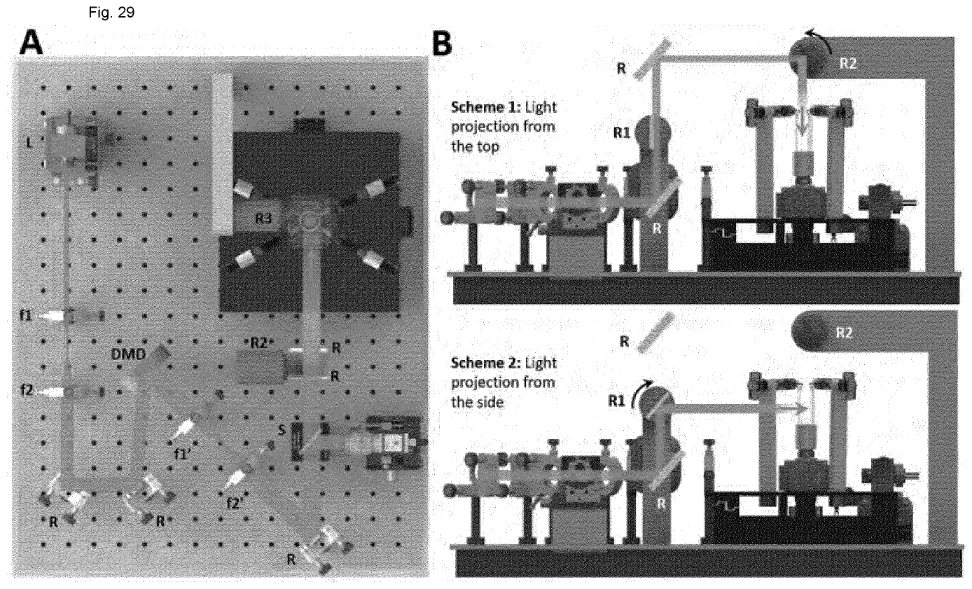

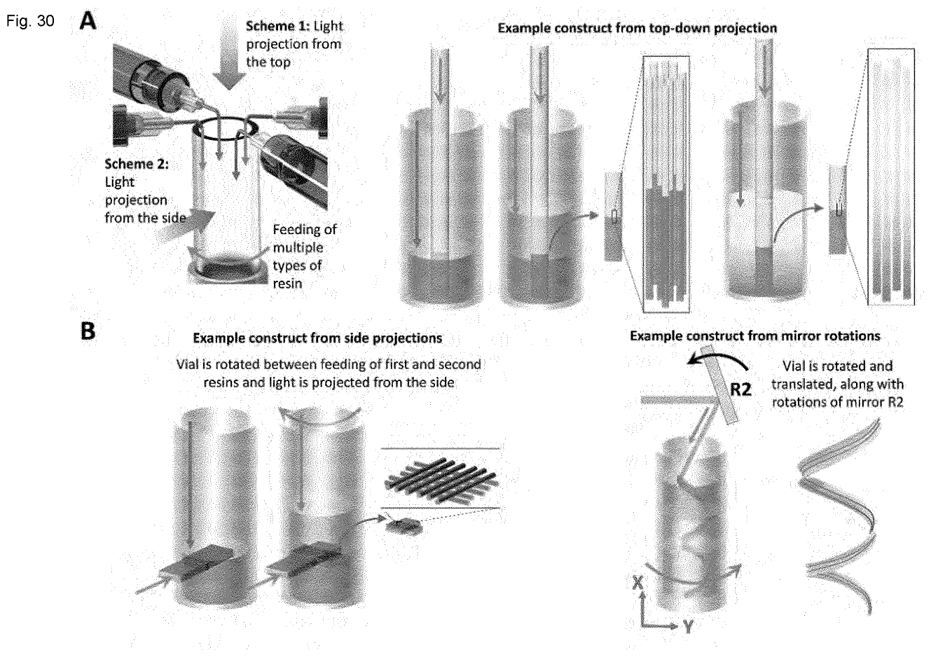

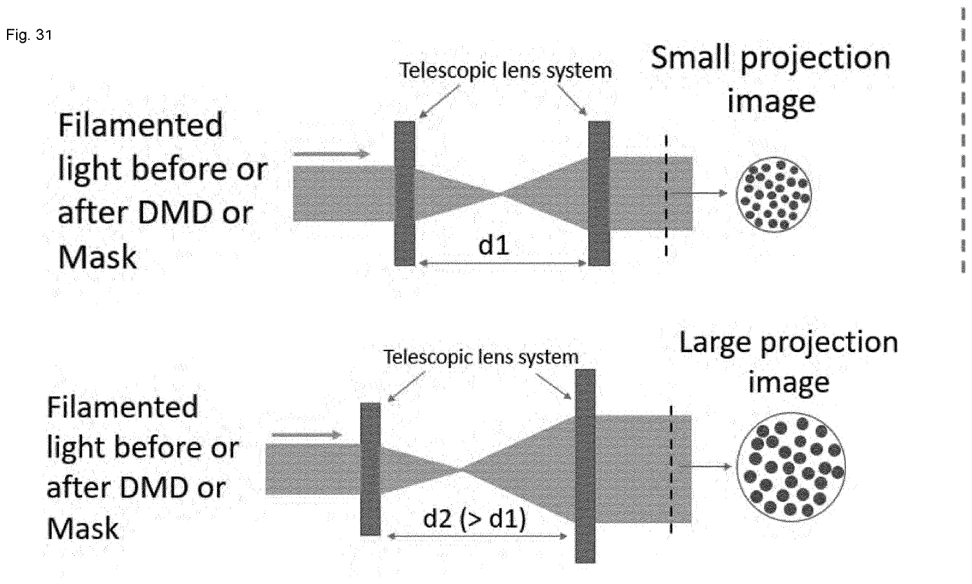

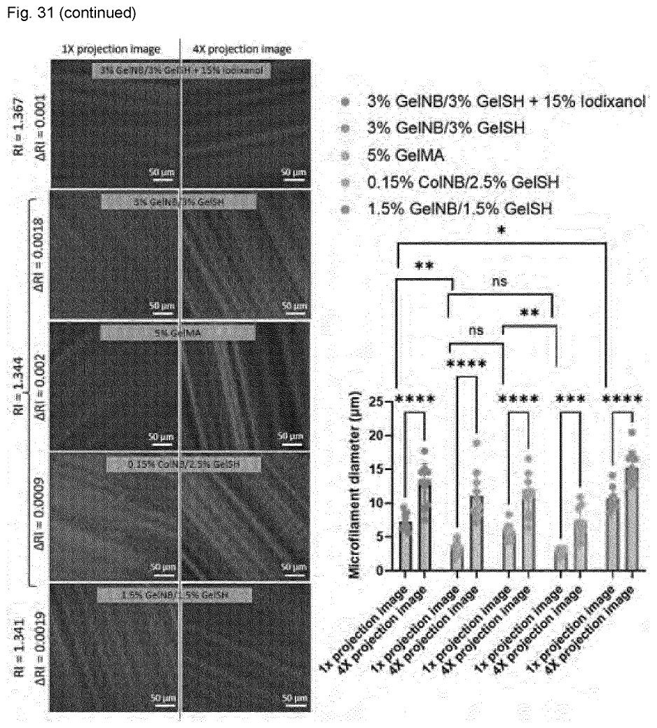

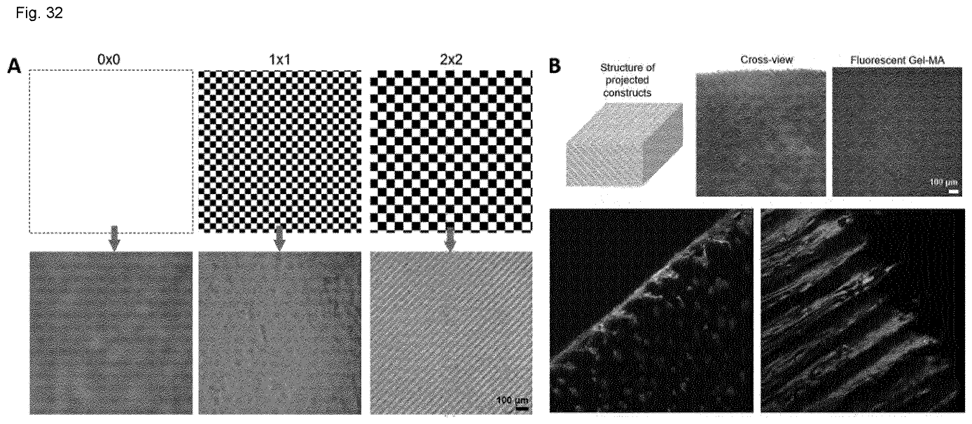

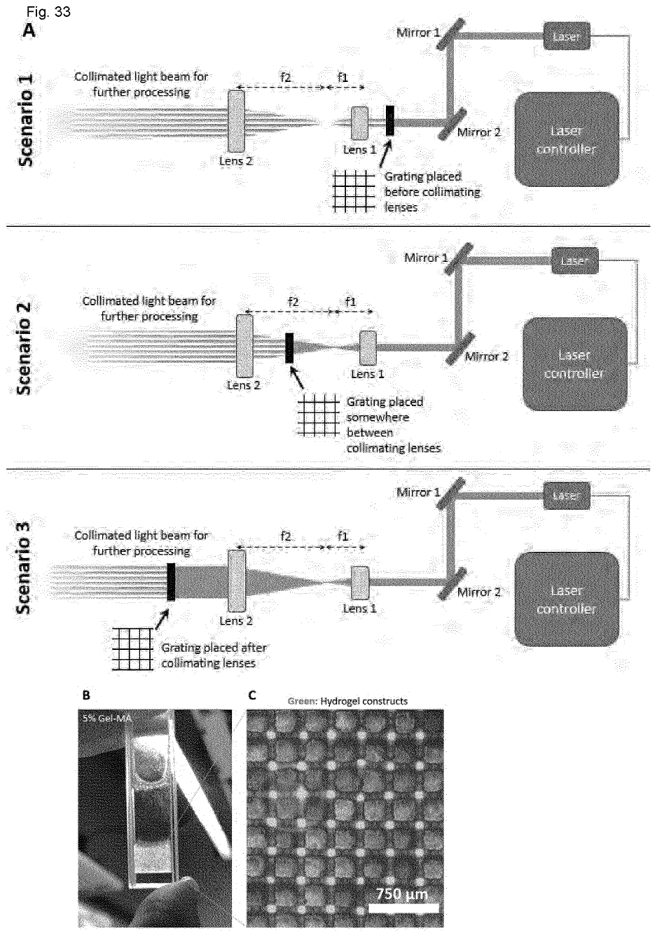

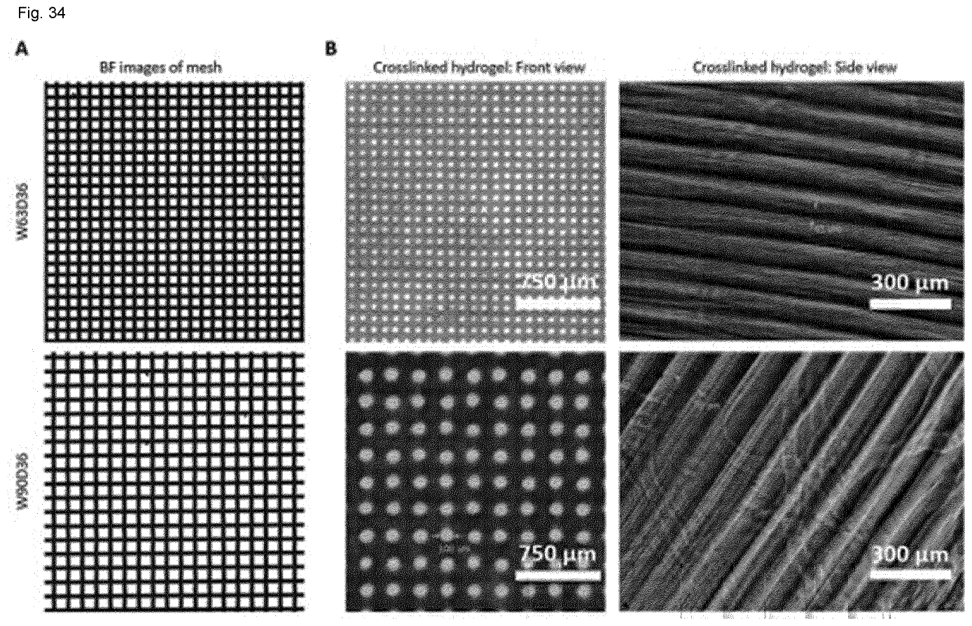

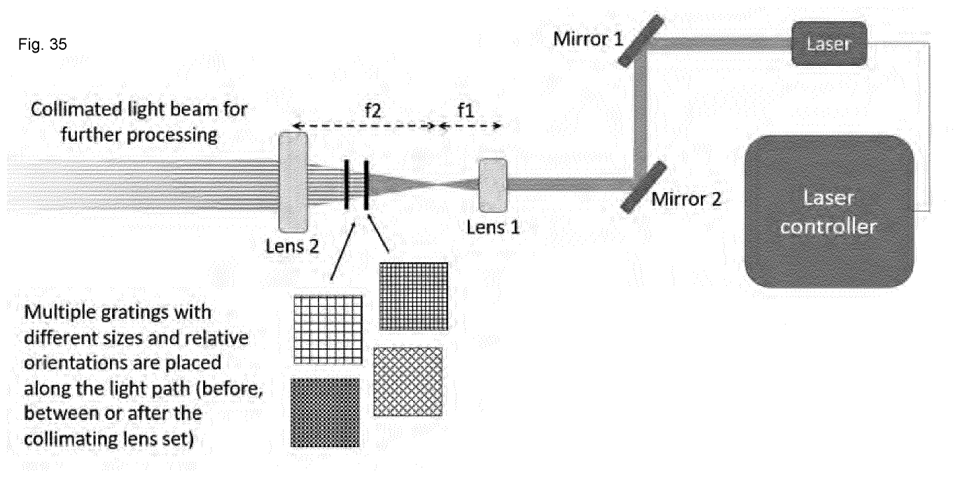

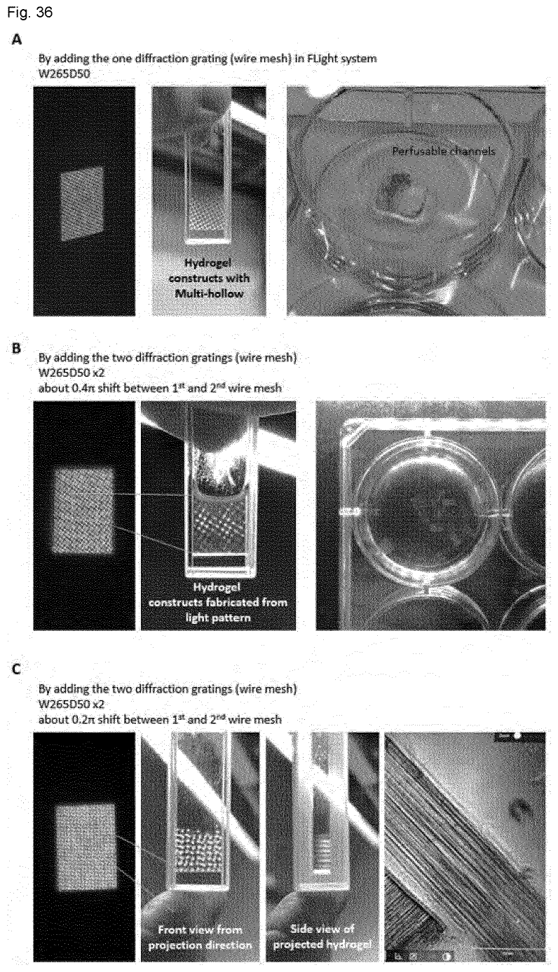

For example, in 5% w/v Gel-NB/Gel-SH photoresin containing 0.05% w/v LAP, 30% w/v iodixanol and C2C12 cells at 10 million cells/mL, the inventors use 50 μg/ml of sunset yellow dye (which is known to absorb 405 nm light) to achieve a 5 mm penetration depth per layer of projection with the prevalence of microbeams (FIG. 26). The resulting constructs (~3 cm long made using 6 projections) feature microbeams throughout their length and formation of aligned myotubes throughout their length. Herein, one can vary the concentration of photo-absorptive dye (1 μg/mL to 500 μg/mL) and lodixanol (5-80% w/v) as per the required needs of the experiment.

### Example 9: Conclusion

The inventors have demonstrated a rapid biofabrication method for creating 3D hydrogel constructs containing cell guiding microbeams. The alignment of four types of cells/deposited ECM was achieved by LBP. The ultra-high-aspect-ratio topological cues provided by the microchannels have a potent effect on cell and nuclear morphology. By performing multiple sequential projection processes, a multi-cellular/multi-material biofabrication strategy was established, which enables mimicking of the hierarchical organization of tissues.

### Example 10A: Grafts for Nerve Repair

Nerve injuries can result in loss of sensation, muscle function, and overall function of the affected limb or body part. Traditional treatments for nerve injuries, such as nerve autografts, have limitations and can result in donor site morbidity. Grafts fabricated using the filamented light (FLight) biofabrication technology can provide a promising alternative for nerve injury repair due to their ability to promote directed axonal growth, and tissue regeneration and integration. FLight grafts with directionally oriented microfilaments can be used for nerve injury repair, for example, to create nerve conduits that provide a scaffolding for directed axon regeneration (acellular grafts for nerve conduits); or as nerve wraps for treatment of compression injury.

The material of the grafts can be based on one or a combination of the resins as laid out above, particularly resins based on step growth photo-polymerization or resins based on chain growth photo-polymerization.

Combinations of cells (such as Schwann cells, human mesenchymal stem cells, human apiose stem cells or induced pluripotent stem cells) may be employed, which have been demonstrated to promote axonal regeneration. The cells will be encapsulated in the grafts.

Growth factors (such as nerve growth factor, neurotropic growth factor) which have been demonstrated to promote axonal regeneration may be present.

In particular embodiments, at least one of the material components in the matrix will be based on collagen or decellularized matrix.

A scheme of the administration of the nerve grafts is described in the following: The grafts are substantially cylindrical in shape, with diameters between 1-7 mm and lengths between 5-50 mm. The grafts can be made to feature different microarchitectural arrangements, such as a fascicular arrangement of microfilaments (φmicrofilaments=1-30 μm) and meshed structures (φmeshes=30-150 μm). Furthermore, by changing the grey-scale of the projected images in the FLight system, grafts featuring different core and shell stiffness can be created. Here, the shell can be created at a higher light intensity such that it has high yield strength to allow suturing/anastomosis, while the core part is made through a lower intensity such that it has lower stiffness (<10 kPa) to promote axonal growth. Both of the core and shell portions will be made to feature uniaxially aligned microfilaments to allow axonal growth. In addition to increasing the light exposure at the shell region, the strength of the shell region can be increased further by secondary crosslinking mechanisms such as chemical crosslinking upon a brief exposure to bacterial transglutaminase (for gelatin or collagen-based resins), or ionic crosslinking (for alginate-based resins).

The yield strength required for nerve grafts to be sutured can depend on various factors such as the type of nerve, the diameter of the nerve, and the tension applied during suturing. Generally, the yield strength of nerve grafts should be high enough to resist the tension applied during suturing without breaking or tearing. The detachment force of nerve grafts, determined using tensile tests will be around 20-60 N, which translates to a yield strength of 10-100 MPa.

The yield strength required for nerve grafts to be sutured varies depending on the application. However, studies have shown that nerve grafts with a yield strength of at least 30-50 N are suitable for suturing. Additionally, nerve grafts with a higher yield strength may provide better outcomes as they are more resistant to stretching or deformation, which can occur during suturing or post-surgical manipulation.

Experiments employing dorsal root ganglions (DRG) encapsulated within the FLight gels have shown that the softer gels (young's modulus 1-3 kPa) with meshes lead to the best axonal growth in terms of the length of axons and the number of axons. Both the length of the axons and the number of axons normalized to the size of the DRGs is higher in softer constructs (~2-3 kPa) featuring meshes (preferably the 63 μm meshes). Microfilaments of the FLight hydrogels can guide the axonal growth both ways (front and back).

### Example 108: Grafts for Muscle Repair

Several types of muscle pathologies lend themselves to treatment with FLight grafts. For example, dermal matrix allografts have been used for the treatment of massive irreparable rotator cuff tears, which can involve significant muscle injury. Similarly, the use of cellular or acellular grafts for the treatment of volumetric muscle loss is a highly prolific research and development area. In the case of muscle injuries, grafts provided herein have the ability to support the attachment, alignment and proliferation of cells, as well as facilitate the formation of new extracellular matrix. Here, grafts featuring different combinations of the resins and materials constituents (preferentially-one of components is based on Collagen or decellularized matrix) depicted in the nerve grafts section would be ideal.

Potential application of FLight grafts include:
1. Muscle strains and tears—Acellular grafts can be used to repair and regenerate muscle tissue that has been damaged by strains or tears. The grafts can provide support and scaffolding for new tissue growth and help to improve muscle function.
2. Muscle atrophy—Muscle atrophy is the loss of muscle mass and strength that can occur due to aging, injury, or disease. Acellular grafts can be used to promote muscle regeneration and prevent further muscle loss in these cases.
3. Muscular dystrophy—Muscular dystrophy is a genetic disease that causes progressive muscle weakness and degeneration. While acellular grafts may not be able to cure the disease, they can be used to improve muscle function and quality of life for patients.
4. Volumetric muscle loss (VML)—VML occurs when a significant portion of muscle tissue is lost due to injury or surgery. Acellular grafts can be used to promote muscle regeneration and prevent the development of scar tissue in these cases.

### Example 10C: Grafts for Tendon Repair

FLight grafts can also used in the treatment of various tendinopathies that have failed to respond to conservative treatment. Here, the aligned microarchitecture of FLight grafts will be important for the treatment of tendons using acellular grafts. Tendons have a highly organized structure, with collagen fibers aligned in a parallel orientation to resist tension and transmit force. Some tendinopathies that can be repaired with FLight grafts include:
1. Achilles tendinopathy
2. Rotator cuff tendinopathy
3. Patellar tendinopathy
4. Tennis elbow (lateral epicondylitis)
5. Golfer's elbow (medial epicondylitis)

In these conditions, FLight grafts can be used to repair or augment the tendon tissue that has been damaged or weakened. They can help to improve tendon strength, reduce pain, and promote healing. The mechanical characteristics (yield strength) required for grafts used in tendinopathy repair can vary depending on the specific application and the patient's individual needs. Generally, the graft should have sufficient strength to support the damaged or weakened tendon and allow for proper healing. For Achilles tendon repair, acellular grafts with a yield strength of at least 250 kPa have been reported to provide good clinical outcomes. For rotator cuff repair, grafts with a yield strength of at least 200 kPa are typically used. For patellar tendinopathy, acellular grafts with a yield strength of at least 150 kPa have been used successfully. For tennis elbow and golfer's elbow, grafts with a yield strength of at least 100 kPa have been reported to provide good results.

### Example 10D: Articular Cartilage Grafts

Articular cartilage repair is necessary in some pathologies because this tissue has limited capacity for self-repair owing to limited vascularity and a highly dynamic growth environment of the joint. Cartilage damage can occur due to a variety of reasons, including traumatic injury, degenerative joint diseases such as osteoarthritis, or genetic conditions such as osteochondritis dissecans.

Injuries to articular cartilage can range from small surface defects to full-thickness cartilage loss. If left untreated, these defects can progress to more severe degenerative joint diseases, leading to pain, loss of joint function, and decreased quality of life.

To use acellular grafts for articular cartilage repair, the damaged cartilage will be first debrided and smoothed to create a stable surface. The graft will be then prepared and sized to fit the defect. The graft will be anchored in place using sutures or tissue glue. Over time, the graft will become incorporated into the surrounding tissue, stimulating the infiltration and growth of new cartilage cells and promoting tissue regeneration.

### Materials and Methods

All chemicals are purchased from Sigma-Aldrich and cell culture reagents from Gibco unless otherwise indicated.

### Synthesis of Gel-NB

The synthesis process of Gel-NB and its characterization were described in the inventors' previous study:^{[5]} briefly, gelatin type A from porcine skin was dissolved at 10% w/v in 0.1 M pH 9 carbonate-bicarbonate buffer at 50° C. Then, ⅕ of the total cis-5-Norbornene-endo-2,3-dicarboxylic anhydride (carbic anhydride, CA) necessary to get the desired Gel:CA ratio was added to the solution. The reaction was left to proceed for 10 minutes under stirring prior to pH adjustment to 9 with NaOH 0.5 M solution. pH adjustment and sequential addition of CA were repeated 5 times every 10 minutes. The solution was then diluted two-fold with Milli-Q water pre-warmed to 40° C. and the pH adjusted to 7.4 with a solution of HCl 0.5 M. Upon centrifugation for 15 minutes at 3000 rcf, the supernatant was then dialyzed at 40° C. against Milli-Q water with frequent water changes for 3-4 days and finally freeze-dried.

For the photoresin preparation, the freeze-dried Gel-NB was dissolved in PBS and kept at 40° C. for 30 minutes. The 4PEG-SH (10 kDa, JenKem Technology) was added to the Gel-NB solution to obtain the desired SH:NB ratio (1:1). Next, a 2% w/v photoinitiator (PI) lithium phenyl-2,4,6-trimethylbenzoylphosphinate (LAP) stock solution in PBS was mixed with Gel-NB/4PEG-SH solution to get a final concentration at 0.05% w/v. The photoresin was filtered by 0.45 μm filter (Filtropur S 0.45, SARSTEDT AG) and stored away from light.

### Synthesis of Gel-MA and Fluorescent-labeled Gel-MA

Gel-MA was synthesized as previously described. The degree of substitution (DS) was estimated with ¹H-NMR (Bruker Ultrashield 400 MHz, 1024 scans) in D20 (Apollo Scientific). Gel-MA lysine integration signal (2.95-3.05 ppm) was compared to unmodified gelatin lysine integration signal (2.95-3.05 ppm). Phenylalanine signal (7.2-7.5 ppm) was used as an internal reference. DS was found to be ~45%.

Fluorescent-labeled Gel-MA was fabricated by modifying the fluorescein-5-isothiocyanate (FITC) or rhodamine B isothiocyanate (RBITC) onto the Gel-MA. Briefly, 10% w/v of Gel-MA was firstly dissolved in 100 mM sodium bicarbonate solution. A 0.1% w/v of FITC-DMF or rhodamine solution was then added, and the mixture was stirred at 40° C. for 6 h in the dark. After the reaction, the mixture was dialyzed against deionized water for 4 days at 30° C. to remove the unreacted monomers with subsequent freeze-drying to obtain fluorescent-labeled Gel-MA.

For the photoresin preparation, the freeze-dried Gel-MA was dissolved in PBS and kept at 40° C. for 30 minutes. Then, a 2% w/v LAP stock solution was mixed with Gel-MA solution to achieve a final concentration of 5% w/v Gel-MA and 0.05% w/v LAP. The photoresin was filtered by 0.45 μm filter and stored away from light.

### Synthesis of HA-MA

Low molecular weight HA-MA (M_{w}=50,000-70,000) was purchased with the DS 20-50%.

For photoresin preparation, HA-MA was dispersed in PBS and stirred at 4° C. until complete dissolution and then mixed with a 2% w/v LAP stock solution to prepare 2% w/v HA-MA and 0.05% w/v LAP solutions. The photoresin was filtered by 0.45 μm filter and stored away from light.

### Synthesis of Alg-MA

Sodium alginate (M_{w}>200 kDa, PRONOVA UP MVG, NovaMatrix®) was dissolved in 25 mL of Milli-Q water to produce 1% w/v solution by continuous stirring. Methacrylic anhydride was distilled prior to use (20 eq. to hydroxyl groups, 52 mmol, 7.7 mL) and added; the formed emulsion was vigorously stirred for 24 h at room temperature. pH was frequently checked and adjusted to 7-8 with NaOH 0.5 M solution. The solution was transferred to Falcon tubes and centrifuged at 3000 rcf for 15 minutes to remove excess of methacrylic anhydride. The aqueous phase was precipitated into ethanol and the precipitate was filtered through a fritted glass funnel (S4 porosity) and dried overnight under high vacuum. Dry Alg-MA was dissolved in Milli-Q water and then dialyzed at RT with frequent water changes for 3-4 days. The Alg-MA was obtained after freeze-drying. ¹H-NMR spectra to determine DS were acquired using 1% w/v Alg-MA in D20 on a Bruker spectrometer operating at a 400 MHz proton frequency. DS was found to be ~51%.

To prepare the photoresin, the freeze-dried Alg-MA was dissolved in PBS. Then a LAP stock solution was mixed with Alg-MA solution to achieve a final concentration of 2% w/v Alg-MA and 0.05% w/v LAP. The photoresin was filtered by 0.45 μm filter before use.

### Cell Culture

Tenocytes were kindly provided by Prof. Lee Ann Applegate and described previously.^{[66]} Human fetal progenitor tenocytes were isolated from the Achilles tendon of a male 14 week gestation organ donation according to a protocol approved by an ethics committee. University Hospital of Lausanne (CHUV), Ethics Committee Protocol No. 62/07: 14-week gestation organ donation, registered under the Federal Transplantation Program and its DAL (Department of Musculoskeletal Medicine) Biobank complying with the laws and regulations. NHDFs were isolated from juvenile foreskin skin biopsies. The biopsies were obtained under parental informed consent and their use for research purposes was approved by the Ethical Committee of Canton Zurich (BASEC-Request-Nr. 2018-00269). Tenocytes and Normal Human Dermal Fibroblasts (NHDFs) were cultured in Falcon® Cell Culture Multi-Flask (TC 5-layer, 875 cm²) with DMEM+GlutaMAX™-I+10% w/v fetal bovine serum (FBS)+10 μg mL⁻¹ Antibiotic-Antimycotic (Anti-Anti). Myoblasts (C2C12) were obtained from ATCC and cultured in DMEM medium+10% v/v FBS+10 μg mL⁻¹ Anti-Anti for cell proliferation. Cells were passaged at 90% confluence and detached using 0.25% Trypsin/EDTA. For C2C12 differentiation, the cell-laden hydrogels were incubated in differentiation medium composed of DMEM+2% v/v horse serum+1% v/v Insulin-Transferrin-Selenium (ITS+, Corning)+10 μg mL⁻¹ gentamicin. Samples were cultured in a 35 mm petri dish (PS 60/15MM, Greiner Bio-One) with frequent medium changes. Human Umbilical Vein Endothelial Cells (HUVEC) were purchased from Lonza and cultured in Endothelial Cell Growth Medium-2 BulletKit (EGM-2, Lonza). For the HUVEC-NHDF co-culture system in hydrogel construct, HUVEC and NHDFs were mixed at a ratio of 1:2 in the photoresin to prepare the biophotoresin. The fabricated cell-laden hydrogel samples were transferred into 6 well plates and cultured in a mixed medium (DMEM: EGM-2 as 1:1).

### Preparation of Biophotoresin

Photoresins were prepared as indicated above. A 0.2 μm filter (Filtropur S 0.2, SARSTEDT AG) was used to sterilize the photoresins and remove potentially scattering particles. The NHDFs and HTs were resuspended in photoresins at a concentration of 1 million cell mL⁻¹. The C2C12 was mixed with photoresins at 2 million cell mL-1. For NHDFs-HUVEC co-culture, two types of cells were mixed at a ratio of 2:1 at a concentration of 1 million cell mL⁻¹ in photoresins.

### Design of Projection Images

Projection images were created using Affinity Photo (AffinitySuite 1.9, Serif Europe Ltd.) with a fixed resolution of 1024×768 pixels. The images were grayscale, with a pure white color corresponding to 100% light intensity (approx. 62.5 mW/cm²). The width of each pixel in the projection image was equal to about 27 μm. The patterns were corrected by 900 as they were projected, with a 900 rotation to the vials and cuvettes. The projection images were then exported as PNG files.

### Assemblage of Standard Köhler Illumination System

All components for assembling a standard Köhler illumination system were brought from Thorlabs if not otherwise clarified, including the light source (M405L4). The assembly and alignment protocols were described in a previous study (J. Madrid-Wolff et al., Biophys. 2020, 1, 10.). Light intensity in front of the cuvette was measured after each adjustment of field diameter and/or aperture diameter using a power meter.

### Light Beam Projection

(Bio)photoresins were prepared as described above and transferred to sterilized cuvettes or glass vials. The Gel-NB/4PEG-SH and Gel-MA (bio)photoresins were allowed to thermally gel at 4° C. for 15 mins. For highly viscous photoresins or non-thermoreversible photoresins (i.e., HA-MA, Alg-MA), the above steps were skipped. Projection was then performed using the “Advanced” built-in function on a commercially available volumetric printer (Tomolite Ver. 1.0, Readily3D SA). The projection times were calculated based on the light intensity and the light doses required for the crosslinking of each photoresin. The projection images were loaded into the software (Apparite, Readily3D SA) before starting the projection. Uncrosslinked (bio)photoresin was washed away using PBS pre-warmed to 37° C. The projected constructs were removed using a sterile spatula and transferred into PBS or culture medium.

### Compression Modulus Measurements

All samples were prepared as cylindrical models with a diameter of 5 mm and height of 4 mm. A circular projection image of 185 pixels in diameter was used to prepare the LBP hydrogel samples. Bulk hydrogel samples (control group) were generated by filling photoresin into PDMS molds (5 mm inner diameter and 4 mm height) before UV crosslinking. Samples were tested by unconfined uniaxial compression using TA.XTplus Texture Analyzer (Stable Micro systems). A 500 g load cell and a flat plate probe (15 mm) were used. A 0.2 g pre-load was applied to ensure that the samples were in full contact with the plates. Samples were compressed to a final strain of 50% at a rate of 0.01 mm s⁻¹. Elastic compressive modulus was calculated by linear fitting of initial linear region (0.5-5%) of the stress-strain curve. The tests were carried out at 25° C. and repeated 3 times.

### Tensile Tests

All samples were prepared as strands with a diameter of 1 mm. A circular matrix (8×8) image of 37 pixels in diameter was loaded to LBP. Bulk hydrogel samples were created by pressing the bulk gel through a grid (1 mm, micron-sized apertures) after thermoreversible gelation of photoresin. Microstrands were then crosslinked under a UV lamp using the same light dose as for the LBP process. Tensile tests were performed by Texture Analyzer equipped with a 500 g load cell using miniature tensile grips and a 0.1 g pre-load was applied before tension. All samples were stretched at a speed of 0.1 mm s⁻¹ until fracture. The loading-unloading curves were measured at a tensile strain of 0-50%, with a tension rate of 0.1 mm s⁻¹ over 12 times cycles.

### Cell Viability

Hydrogel constructs were washed three times with PBS after 0, 3, 7 days of culture and then incubated for 45 minutes in FluoroBrite™ DMEM supplemented with 1:2000 CalceinAM (Invitrogen), 1:1000 Hoechst 33342 (Invitrogen), and 1:500 Propidium Iodide (PI, Fluka). Fluorescent imaging was performed with a confocal laser scanning microscope (Fluoview 3000, Olympus) after three times washing in medium. Z-stacks scanning was acquired from the surface of constructs at 5 μm steps 200 μm into the constructs. The z-projection images were analyzed with Fiji ImageJ. The above experiment was repeated three times, with the viability of each sample averaged over three images of randomly chosen areas.

### CellTracker Dye Loading

CellTracker Green dye CMFDA (Invitrogen) and CellTracker Red dye CMTPX were applied to NHDFs in serum-free medium at a working concentration of 10 μM. Cells were transferred to the incubator at 37° C. for 1 hour and then washed with PBS three times for subsequent experiments.

### Immunofluorescence Staining

Cell-laden hydrogel strands were washed with PBS three times after 7 days of culture and fixed in 4% paraformaldehyde for 30 mins at 25° C. The strands were permeabilized with 0.2% Triton-X100 in PBS for 30 mins before blocking with 1% v/v BSA in PBS for 1 hour. The strands were then incubated with primary anti-human collagen I antibody (ab138492, Abcam) 1/500 diluted in BSA-PBS at 4° C. for 12 hours. Next, the samples were washed 3 times with PBS, incubated with 1/200 diluted secondary antibody (Goat anti-Rabbit Alexa Fluor 488, Invitrogen), Hoechst 33342, and pre-prepared Phalloidin-Tetramethylrhodamine B isothiocyanate working solution (0.13 μg mL⁻¹, P1951) in BSA-PBS for 2 h at 4° C. C2C12 constructs were incubated with 1/20 diluted primary anti-human myosin heavy chain antibody (MF-20, DSHB) and 1/500 diluted collagen I antibody for 12 hours at 4° C. The samples were then incubated with 1/200 diluted secondary antibody (Goat anti-Rabbit Alexa Fluor 647, Goat anti-Mouse Alexa Fluor Plus 488), Phalloidin-Tetramethylrhodamine B isothiocyanate working solution and Hoechst for 2 hours at 4° C. Samples were washed with PBS before imaging on CLSM. NHDFs encapsulated in tubular strands, encapsulated in Fluorescence GelMA, or seeded on the surface were stained by CalceinAM for 1 h. Tubular constructs were then perfused with 40 kDa tetramethylrhodamine isothiocyanate-dextran (TRITC-dextran) for 10 mins before confocal imaging.

RNA Isolation & qRT-PCR

NHDFs were encapsulated into hydrogel samples fabricated using 2.88% w/v Gel-NB/4PEG-SH photoresin. The tissue samples were homogenized with tissue grinders and incubated with NucleoZOL (MACHEREY-NAGEL) at room temperature for 10 mins. DNase-free water was added to the samples and mixtures were centrifugated for 10 mins at 12000 rcf. The supernatants were mixed with 70% EtOH and transferred to the RNeasy mini kit (Qiagen) column to extract the total RNA. An A260/280 ratio of between 1.8 to 2.1 was accepted as adequate quality for the RNA samples. The isolated RNA was transcribed to complementary DNA following the instruction of GoScript Reverse Transcriptase kit (Promega). The relative gene expression levels were determined on Real-Time PCR System (QuantStudio 5, Applied Biosystems) with the SYBR Green PCR Master Mix (Promega). The GAPDH housekeeper gene was used as an internal control for the normalization of RNA levels.

### 3D-Image Reconstruction

The 3D images obtained using confocal microscopy were imported into Imaris 9.2.1 for reconstruction of the 3D model. The measurements in this work were performed by surface and statistic functions.

### Statistical Analysis

Statistical analysis was performed using GraphPad Prism (x64, v. 9.2.0) and unpaired t-tests. Alpha was set to 0.05 and differences between two experimental groups were judged to be statistical significance at *p<0.05, *p<0.05, **p<0.01, and ***p<0.005; and ns represents “no significant difference” between two groups.

### CITED PRIOR ART DOCUMENTS

Göckler et al., Advanced Healthcare Materials 19 Jun. 2021;
Guo et al., ACS Appl. Mater. Interfaces 2021, 13, 6, 7037-7050;
Michel et al., ACS Appl. Bio Mater. 2020, 3, 8, 5253-5262;
M. Veettikazhy et al., Opt. Express 2021, 29, 11819;
M. Lee, et al., Chem. Rev. 2020, 120, 10950.
J. Madrid-Wolff, M. Forero-Shelton, Biophys. 2020, 1, 10.

1. A method for making a three-dimensional hydrogel bioimplant comprising
a. providing a first composition susceptible to photo-crosslinking in a container, said first composition comprising
i. a first polymer susceptible to photo-crosslinking,
ii. a photoinitiator and
iii. optionally, a refractive index matching agent (e.g., iodixanol up to 80% w/v, particularly 10% to 50%) which can increase or decrease the refractive index of photoresin formula
iv. optionally, photo absorptive dye (e.g., Sunset Yellow or FCF yellow at concentration up to 500 μg/mL)
v. optionally, a crosslinking agent and
b. in a first illumination step, illuminating the composition with a plurality of spatially coherent light beams,
thereby generating a plurality of micropillars in the composition.

2. The method according to claim 1, wherein said spatially coherent light beams are characterized by a diameter of 1 to 100 μm, particularly by a diameter from 3 μm to 40 μm.

3. The method according to claim 1, wherein
a. the composition is illuminated by a first plurality of spatially coherent light beams aligned in a first direction, and
b. wherein the composition is illuminated, in a second illumination step, by a second plurality of spatially coherent light beams aligned in a second direction.

4. The method according to claim 3, wherein said first direction and said second direction are arranged at an angle of 10 to 180°, particularly 15° to 180°.

5. The method according to claim 3, wherein
said first plurality of spatially coherent light beams forms a first pattern;
said second plurality of spatially coherent light beams forms a second pattern; and
said first and/or said second pattern are arranged in cylindrical form, in form of a sheet, or as parallel strands between anchoring tendon-like support sheets.

6. The method according to claim 1, wherein subsequent to said first illumination step, a second composition is added to the container, said second composition comprising
i. a second polymer susceptible to photo-crosslinking,
ii. a photoinitiator and
iii. optionally, a refractive index matching agent (e.g., iodixanol up to 80% w/v) which can increase or decrease the refractive index of photoresin formula;
iv. optionally, photo absorptive dye (e.g., Sunset Yellow or FCF yellow at concentration up to 500 μg/mL)
v. optionally, a crosslinking agent and
in a third illumination step, or any subsequent illumination step, illuminating the composition with a plurality of spatially coherent light beams.

7. The method according to claim 1, wherein in the first, second, third or any subsequent illumination step, illumination is effected from top or bottom side of the container onto a substrate; the continuous illumination is performed from the top of the container, or the substrate is moved upwards away from the bottom of the container under continuous illumination, thereby forming a plurality of microbeams protruding from the substrate, in which microbeams prevalent through the length of the hydrogel constructs in a longitudinal axis.

8. The method according to claim 1, wherein the polymer susceptible to photo-crosslinking is a biopolymer functionalized by covalent attachment of carbon-carbon double bond (ene) containing moieties or a methacrylate-functionalized biopolymer, particularly a biopolymer is selected from the group of gelatin, hyaluronan, alginate, collagen, fibrinogen, polyvinyl alcohol, chitosan, silk fibroin, cellulose.

9. (canceled)

10. The method according to claim 8, wherein the carbon-carbon double bond (ene) containing moieties are selected from the group comprised of a norbornene carboxylic acid or dicarboxylic acid, methacrylic acid ester or -amide, acrylic acid ester or -amide, and vinyl esters.

11. The method according to claim 8, wherein the polymer susceptible to photo-crosslinking is norbornene-functionalized gelatin characterized by a degree of substitution of 10% to 90%, particularly by a degree of substitution from 47% to 50%, or is methacrylate-functionalized gelatin characterized by a degree of 10% to 90%, particularly from 45-60%.

12. (canceled)

13. The method according to claim 1, wherein the spatially coherent light beam is characterized by a wavelength of 360 to 800 nm, or by a coherence length of 1 to 100 μm, or by a light dose of 10 to 5000 mJ/cm², particularly of 90 to 200 mJ/cm².

14. (canceled)

15. (canceled)

16. The method according to claim 1, wherein the spatially coherent light beam is characterized by a duration of 0.1 s to 100 s, particularly from 1 to 4 s.

17. The method according to claim 1, wherein the spatially coherent light beam is characterized by an energy density of 1 to 500 mW/cm², particularly of 50 to 60 mW/cm².

18. A three-dimensional hydrogel implant comprising or essentially consisting of a first plurality of micropillars of a first photo-crosslinked polymer, wherein said three-dimensional hydrogel implant was obtained by a method according to claim 1.

19. The three-dimensional hydrogel bioimplant according to claim 18, wherein said first plurality of micropillars is characterized by each of the microbeams having a diameter ranging from 1 μm to 100 μm.

20. The three-dimensional hydrogel bioimplant according to claim 18, wherein said first plurality of micropillars is characterized by each of the microbeams having a diameter ranging from 2 μm to 30 μm

21. The three-dimensional hydrogel bioimplant according to claim 18, wherein >75% (particularly >80%, ≥85%, ≥90%, ≥95% or even ≥98%) of said microbeams of said plurality having an alignment of ≤2° deviation from a longitudinal axis.

22. The three-dimensional hydrogel bioimplant according to claim 18, wherein >75% (particularly >80%, ≥85%, ≥90%, ≥95% or even ≥98%) of said microbeams of said plurality having a length of >2 cm, particularly ≥5 cm.

23. The three-dimensional hydrogel bioimplant according to claim 18, wherein microbeams are prevalent through the length of the hydrogel constructs in a longitudinal axis.

24. The three-dimensional hydrogel bioimplant according to claim 1, wherein said plurality of microbeams forms channel structures having a diameter of 100 μm to 10 mm, particularly by a diameter of 400 μm to 600 μm.
